SEQUENCE LISTING (3 sequences)

SEQ ID NO: 1 - RNA, 71 nt, Artificial Sequence
  miR-30a precursor
gcgacuguaaacauccucgacuggaagcugugaagccacagaugggcuuucagucggauguuugcagcugc

SEQ ID NO: 2 - DNA, 21 bp, Artificial Sequence
  siRNA-p sequence
uggaagcugugaagccacatt

SEQ ID NO: 3 - DNA, 21 bp, Artificial Sequence
  siRNA-c sequence
ugguagcagucauggcacatt

(19) United States
(12) Patent Application Publication
(10) Pub. No.: US 2012/0071539 A1
(43) Pub. Date: Mar. 22, 2012

(54) COMPOUNDS AND METHODS FOR MODULATING THE SILENCING OF A POLYNUCLEOTIDE OF INTEREST

(75) Inventor: Peng Jin, Marietta, GA (US)

(73) Assignee: Emory University, Atlanta, GA (US)

(21) Appl. No.: 12/519,062

(22) PCT Filed: Dec. 12, 2007
(86) PCT No.: PCT/US2007/087186

§ 371 Date: Nov. 18, 2011

Related U.S. Application Data
(60) Provisional application No. 60/874,381, filed on Dec. 12, 2006.

(51) Int. Cl. A61K 31/713; A61K 31/4439; A61K 31/352; A61K 31/575; A61K 31/192; A61K 31/366; A61K 31/16; A61K 31/52; A61K 31/357; A61K 31/395; A61K 31/4985; A61K 31/7105; A61K 8/60; A61K 31/365; A61P 31/12; A61P 43/00; A61K 31/496; C12Q 1/68; C40B 30/06; A61K 31/444; A61K 31/4409; A61P 35/00; A61P 35/02; A61P 25/28; A61P 25/16; C12N 5/071

(52) U.S. Cl. 514/44 A; 435/375; 514/343; 514/450; 514/182; 514/570; 514/453; 514/616; 514/263.37; 514/452; 514/468; 514/250; 514/455; 514/470; 514/253.04; 435/6.13; 506/10; 514/334; 514/332; 514/354

(57) ABSTRACT

Methods and compositions comprising chemical compounds that modulate the silencing of a polynucleotide of interest in a cell are provided. Such chemical compounds when used in combination with an appropriate silencing element can be used to modulate (increase or decrease) the level of the polynucleotide targeted by the silencing element. Methods of using such compositions both in therapies involving RNAi-mediated suppression of gene expression, as well as, in vitro methods that allow for the targeted modulation of expression of a polynucleotide of interest are provided. Pharmaceutical or cosmetic compositions comprising such compounds and silencing elements also are disclosed. Methods for screening a compound of interest for the ability to modulate the activity of a heterologous silencing element also are provided.

### FIELD OF THE INVENTION

The presently disclosed subject matter generally relates to methods and compositions for modulating the silencing of a polynucleotide of interest in a cell. More particularly, the presently disclosed subject matter relates to the use of small molecules, which when used in combination with silencing elements modulate expression of a polynucleotide of interest.

### BACKGROUND

Gene regulation by RNA interference (RNAi) has been recognized as one of the major regulatory pathways in eukaryotic cells. See Plasterk (2006) Cell 124 (5): 877-81. Long double-stranded RNAs (dsRNAs) can be used to suppress or silence the expression of target genes in a variety of organisms and cell types. Upon introduction into an organism or a cell, the long dsRNAs enter a natural cellular pathway, referred to as the RNA interference (RNAi) pathway, wherein the dsRNA is degraded by the cytoplasmic nuclease Dicer. Dicer cleaves the long dsRNA into 20 to 25 base pair (bp) small interfering RNAs (siRNAs), which then unwind and assemble into RNA-induced silencing complexes (RISCs). The antisense siRNA strand then guides the RISC to complementary RNA molecules, wherein the RISC cleaves the target mRNA, leading to specific gene silencing. If the complementary RNA is not perfect, the RISC may only bind to the mRNA, which also blocks translation, thereby inhibiting expression.

Two types of small regulatory RNAs that regulate gene expression have been identified: small interference RNAs (siRNAs) and micro RNAs (miRNAs). Referring now to FIG. 1, in animals, siRNAs direct target messenger RNA (mRNA) cleavage, i.e., mRNA degradation, whereas miRNAs block target mRNA translation, i.e., translation suppression. Thus, in animals, RNAi operates through two post-transcriptional mechanisms: targeted mRNA degradation (siRNA) and the suppression of translation (miRNA). In animals, an RNAi pathway in which silencing occurs by a process involving cleavage of a target transcript mediated by a short RNA that binds to a target transcript to form a duplex structure is referred to as an siRNA RNAi pathway. An RNAi pathway in which silencing occurs by a process involving translational suppression mediated by a short RNA that binds to a target transcript to form a duplex structure is referred to as an miRNA translational suppression pathway. Recent data suggest that siRNAs and miRNAs incorporate into similar protein complexes and that a critical determinant of mRNA degradation versus translation regulation is the degree of sequence complementary between the small RNAs and their mRNA target.

RNAi is a powerful method for the study of gene function in animals and plants. RNAi by small dsRNAs is highly specific because only mRNAs with sequences complementary to the interfering RNA are degraded or blocked. RNAi can be induced by endogenous dsRNA, as well as by exogenous siRNA, for example, after transfection of synthetic siRNA molecules. This feature allows the function of a gene by the selective abrogation of its transcript (i.e., gene knock-down) to be studied through RNAi. Because RNAi technology involves natural cellular mechanisms, RNAi technology can be more efficient than the artificial antisense RNA approach that has failed in numerous experimental settings. Further, because most mammalian cells initiate a potent antiviral response upon introduction of dsRNAs longer than about 30 bp, siRNAs ranging from about 20 bp to about 25 bp typically are used to induce RNAi in mammalian systems without eliciting an antiviral response.

Although RNAi is a relatively new technique, its potential therapeutic applications are significant and far-reaching. The RNAi mechanism has been co-opted by researchers and has achieved broad utility in gene-function analysis, drug-target discovery and validation, and therapeutic development. See Dykxhoorn and Lieberman (2005) Annu Rev Med 56: 401-23; Dykxhoorn and Liebermann (2006) Annu Rev Biomed Eng 8: 377-402. More particularly, RNAi represents a promising therapeutic approach for diseases that result from aberrant protein synthesis. For example, RNAi has been used as a therapy for treating genetic disorders and viral infections. Because RNAi can be used to target virtually any protein, RNAi-based therapies can be developed for almost any therapeutic area. In this way, inhibiting or eliminating a target mRNA would result in a significant decrease in the expression level of a specific protein, thereby serving as a powerful therapeutic tool. Therapeutics based on RNAi potentially have significant advantages over current disease treatments, including, but not limited to, broad applicability, high therapeutic specificity, and target RNA destruction resulting in a decrease or termination of disease progression.

Due to its importance in endogenous gene regulation and its use as a research tool, the RNAi mechanism has been extensively studied for the past several years. Although the major components of the endogenous RNAi machinery have been identified, little is known about the regulation of the RNAi pathway itself. Thus, the mechanisms involved in RNAi, remain poorly understood. For example, many of the molecular components that mediate RNAi remain unidentified. In addition, synthetic siRNAs are easily degraded by RNase before entering the target cell and before exerting their silencing functionalities. Thus, the design and delivery of interfering RNAs to efficiently knock down endogenous genes have been a challenge in the art. To fully exploit the potential of RNAi there is a need in the art for reagents and methods that can be used to identify molecules that can influence the efficacy of RNAi. Methods known in the art involve stabilizing synthetic siRNAs against RNase degradation to achieve higher efficacy extracellularly. The effects of such methods, however, are limited. Therefore, there is a need in the art for molecules that are capable of modulating the suppressive effect of interfering RNA on gene expression, that is molecules that can regulate, control, or modulate, e.g., enhance or inhibit RNAi pathways, and for tools that allow identification of such molecules. The presently disclosed subject matter addresses, in whole or in part, these and other needs in the art.

### SUMMARY

In some embodiments, the presently disclosed subject matter provides a method for modulating the silencing of a target polynucleotide by RNAi inside a cell. The method comprises administering to a cell a reagent comprising a heterologous silencing element, which decreases the level of a target polynucleotide when inside the cell, and an effective amount of at least one modulator. In some embodiments, the presently disclosed modulator is an RNAi enhancer, which increases the silencing element's ability to decrease the level of a target polynucleotide when inside the cell. In some embodiments, the presently disclosed RNAi enhancer comprises at least one compound of Formula (a)-(k) or a metal chelating agent, such as an iron chelating agent, which, in some embodiments, includes a compound of Formula (a)-(q), as disclosed herein.

In some embodiments, at least one compound of Formula (a)-(k) or a metal chelating agent, such as an iron chelating agent, which, in some embodiments, includes a compound of Formula (n)-(q), is administered in combination with another RNAi enhancer, such as RNAi-E, i.e., enoxacin. In such embodiments, the at least one compound of Formula (a)-(k) or a metal chelating agent, such as an iron chelating agent, which, in some embodiments, includes a compound of Formula (n)-(q), modulates, e.g., enhances, the activity of RNAi-E, i.e., it enhances the ability of RNAi-E to increase the silencing element's ability to decrease the level of a target polynucleotide when inside the cell.

In some embodiments, the presently disclosed modulator is an RNAi inhibitor, which decreases the silencing element's ability to decrease the level of a target polynucleotide when inside the cell. In some embodiments, the presently disclosed RNAi inhibitor comprises at least one compound of Formula (l)-(m) as disclosed herein. In some embodiments, at least one compound of Formula (l)-(m) is administered in combination with an RNAi enhancer, such as RNAi-E, i.e., enoxacin. In such embodiments, the at least one compound of Formula (l)-(m) inhibits the activity of RNAi-E, thereby modulating siRNA function.

In some embodiments, the presently disclosed subject matter provides a method for decreasing the level of a target polynucleotide in a cell, the method comprising administering to the cell an effective amount of a compound of Formula (a)-(k), or a derivative or analog thereof, or a metal chelating agent, such as an iron chelating agent, which, in some embodiments, includes a compound of Formula (n)-(q), wherein the cell further comprises at least one heterologous silencing element. In some embodiments, the presently disclosed subject matter provides a method for suppressing RNAi suppression in a cell and thereby decreasing the activity of a silencing element. The method comprises administering to a cell having a silencing element an effective amount of a compound of Formula (l)-(m) as disclosed herein.

In some embodiments, the presently disclosed subject matter provides a pharmaceutical or cosmetic composition comprising a polynucleotide comprising a silencing element, at least one modulating compound, and a pharmaceutically or cosmetically acceptable carrier. More particularly, in some embodiments, the presently disclosed subject matter provides a pharmaceutical or cosmetic composition comprising one or more polynucleotide comprising a silencing element, which, when administered to the cell, decreases the level of a target polynucleotide; a compound of Formula (a)-(k) or a metal chelating agent, such as an iron chelating agent, which, in some embodiments, includes a compound of Formula (n)-(q), as disclosed herein; and a pharmaceutically or cosmetically acceptable carrier. In some embodiments, the presently disclosed subject matter provides a pharmaceutical or cosmetic composition comprising one or more polynucleotide comprising a silencing element, which, when administered to the cell, decreases the level of a target polynucleotide; a compound of Formula (l)-(m); and a pharmaceutically or cosmetically acceptable carrier.

In some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a compound of Formula (a)-(k), or a metal chelating agent, such as an iron chelating agent, which, in some embodiments, includes a compound of Formula (n)-(q), as disclosed herein, either alone or in combination with another RNAi enhancer, and pharmaceutically or cosmetically acceptable salts thereof.

In some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a compound of Formula (l)-(m), as disclosed herein, alone or in combination with an RNAi enhancer, and pharmaceutically or cosmetically acceptable salts thereof.

In some embodiments, the presently disclosed subject matter provides a method for screening a compound of interest for the ability to modulate the activity of a heterologous silencing element, the method comprising: (a) providing a host cell that stably expresses a reporter gene, wherein said host cell further comprises at least one heterologous silencing element capable of inhibiting the expression of the reporter gene; (b) administering to the cell a compound of interest; and (c) measuring the expression of the reporter gene.

The presently disclosed modulating agents represent a novel way to improve the efficiency of RNA interference and gene knock-down. This finding has clinical applications as it improves the efficiency of gene knock-down for RNAi-mediated therapeutic intervention.

Accordingly, it is an object of the presently disclosed subject matter to provide compounds and methods for modulating RNA interference and gene knock-down. It is another object of the presently disclosed subject matter to provide compounds and methods for modulating, i.e., enhancing or inhibiting, RNA interference and gene knock-down intracellularly. It is another object of the presently disclosed subject matter to use the presently disclosed compounds of Formula (a)-(k) or a metal chelating agent, such as an iron chelating agent, which, in some embodiments, includes a compound of Formula (n)-(q), to enhance RNA interference and gene knock-down. It is another object of the presently disclosed subject matter to provide a pharmaceutical or cosmetic composition comprising at least one compound of Formula (a)-(k) or a metal chelating agent, such as an iron chelating agent, which, in some embodiments, includes a compound of Formula (n)-(q), a pharmaceutically or cosmetically acceptable carrier, and, at least one polynucleotide comprising a silencing element, which, when administered to the subject, decreases the level of a target polynucleotide.

It is another object of the presently disclosed subject matter to administer at least one compound of Formula (a)-(k) or a metal chelating agent, such as an iron chelating agent, which, in some embodiments, includes a compound of Formula (n)-(q), in combination with another RNAi enhancer, such as RNAi-E, i.e., enoxacin, to enhance the ability of RNAi-E to increase the silencing element's ability to decrease the level of a target polynucleotide when inside the cell.

It is another object of the presently disclosed subject matter to use a compound of Formula (l)-(m) to suppress RNA interference mRNA degradation. It is another object of the presently disclosed subject matter to provide a pharmaceutical or cosmetic composition comprising a compound of Formula (l)-(m), a pharmaceutically or cosmetically acceptable carrier, and one or more polynucleotide comprising a silencing element, which, when administered to the subject, decreases the level of a target polynucleotide.

It is another object of the presently disclosed subject matter to administer at least one compound of Formula (l)-(m) in combination with an RNAi enhancer, such as RNAi-E, i.e., enoxacin, to inhibit the activity of RNAi-E and thereby modulate siRNA function.

It is another object of the presently disclosed subject matter to provide a method for treating a disease state or unwanted condition.

It is another object of the presently disclosed subject matter to provide a method for screening a compound of interest for the ability to modulate the activity of a heterologous silencing element.

Certain objects of the presently disclosed subject matter having been stated hereinabove, which are addressed in whole or in part by the presently disclosed subject matter, other objects will become evident as the description proceeds when taken in connection with the accompanying Examples and Drawings as best described herein below.

### BRIEF DESCRIPTION OF THE DRAWINGS

Having thus described the invention in general terms, reference will now be made to the accompanying Drawings, which are not necessarily drawn to scale, and wherein:

FIG. 1 (prior art) shows a schematic representation of the common biological pathway of gene knock-down mechanisms involving siRNA-mediated mRNA degradation and miRNA-mediated translation suppression.

FIG. 2 shows a schematic representation of the development of the presently disclosed reporter system for the chemical screening for compounds that modulate siRNA-mediated mRNA degradation and gene knock-down.

FIG. 3 shows the identification of an inhibitor of siRNA-mediated mRNA degradation and gene knock-down.

FIG. 4 shows enhancement of mRNA-mediated mRNA degradation and gene knock-down by a presently disclosed quinolone compound, e.g., enoxacin.

FIG. 5 shows enhancement of mRNA-mediated mRNA degradation and gene knock-down by presently disclosed quinolone compounds, e.g., enoxacin, ciproflaxin, and ofloxacin.

FIG. 6 shows a schematic representation of a microRNA (miRNA) sensor in mammalian cells. (A) Design of a siRNA duplex against miR-30a precursor (siRNA-p) and a control siRNA duplex (siRNA-c). The miR-30a precursor is provided as SEQ ID NO:1. The siRNA target region is represented by nucleotides 21-40 of SEQ ID NO:1. The siRNA-p is provided as SEQ ID NO:2 and the siRNA-c is provided as SEQ ID NO:3. The double stranded siRNA is shown on the bottom and predicted pairing between one siRNA strand (blue) and the precursor target (green).

FIG. 7 shows the relative miRNA-mediated suppression exhibited by a presently disclosed RNAi inhibitor (RNAi-I) as compared to “no drug” and enoxacin.

FIG. 8 shows the suppression of the translation of Lin28 by the expression of miR-125a, wherein the addition of RNAi-E further enhances the suppression.

FIGS. 9A and 9B show the regulation of ferritin translation and transferrin receptor (TfR) mRNA degradation by iron regulation protein (IRP) binding. FIG. 9A shows that in iron-depleted cells, IRP binding to the iron responsive element (IRE) in the 5′ untranslated region (UTR) interferes with translation initiation. FIG. 9B shows that binding of IRPs to IREs in the TfR 3′UTR protects the transcript from endonucleolytic cleavage and degradation. The arrow marks the site at which an unknown endonuclease cleaves the TfR transcript in iron-replete cells when it is unprotected by IRP binding.

FIGS. 10A-10D show chemical structures of representative metal/Fe chelators found to exhibit RNAi-enhancing activity.

FIGS. 11A-11D show drug dose-responses for representative metal/Fe chelators. RNAi-293-EGFP/RFP cells were treated with each compound for 48 hours and the GFP/RFP signal ratio was plotted for relative RNAi-enhancing activity (%) using E50 (Enoxacin at 50 μM) as 100% activity.

### DETAILED DESCRIPTION

The presently disclosed subject matter now will be described more fully hereinafter with reference to the accompanying Drawings, in which some, but not all embodiments of the presently disclosed subject matter are shown. Indeed, the presently disclosed subject matter can be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims. Although specific terms are employed herein, they are used in a generic and descriptive sense only and not for purposes of limitation.

Throughout the specification and claims, a given chemical formula or name shall encompass all optical and stereoisomers, as well as racemic mixtures where such isomers and mixtures exist.

All publications, patent applications, patents, and other references mentioned herein are incorporated by reference in their entirety. It will be understood that, although a number of patent applications, patents, and other references are referred to herein, such reference does not constitute an admission that any of these documents forms part of the common general knowledge in the art.

Throughout this specification and the claims, the words “comprise,” “comprises” and “comprising” are used in a non-exclusive sense, except where the context requires otherwise.

### I. Definitions

Unless otherwise defined, all technical and scientific terms used herein have the same meaning as commonly understood by one of ordinary skill in the art to which this presently described subject matter belongs.

Following long-standing patent law convention, the terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

As used herein, the term “about,” when referring to a value is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

The term “complementary” is used herein in accordance with its art-accepted meaning to refer to the capacity for precise pairing via hydrogen bonds (e.g., Watson-Crick base pairing or Hoogsteen base pairing) between two nucleosides, nucleotides or nucleic acids, and the like. For example, if a nucleotide at a certain position of a first nucleic acid is capable of stably hydrogen bonding with a nucleotide located opposite to that nucleotide in a second nucleic acid, when the nucleic acids are aligned in opposite 5′ to 3′ orientation (i.e., in anti-parallel orientation), then the nucleic acids are considered to be complementary at that position (where position may be defined relative to either end of either nucleic acid, generally with respect to a 5′ end). The nucleotides located opposite one another can be referred to as a “base pair.” A complementary base pair contains two complementary nucleotides, e.g., A and U, A and T, G and C, and the like, whereas a noncomplementary base pair contains two noncomplementary nucleotides (also referred to as a mismatch). Two polynucleotides are said to be complementary to each other when a sufficient number of corresponding positions in each molecule are occupied by nucleotides that hydrogen bond with each other, i.e., a sufficient number of base pairs are complementary.

The compositions disclosed herein, such as the presently disclosed compounds of Formula (a)-(k) and Formula (l)-(m), and the various metal chelating agents disclosed herein, which, in some embodiments, include compounds of Formula (n)-(q), and the one or more polynucleotide comprising a silencing element are used in combination. The term “combination” is used in its broadest sense and means that a cell or a subject is administered at least two agents, more particularly a chemical compound disclosed herein and a silencing element of interest. In some embodiments, at least one compound of Formula (a)-(k) or Formula (n)-(q) can be administered in combination with an RNAi enhancer, such as RNAi-E, i.e., enoxacin. In some embodiments, at least one compound of Formula (l)-(m) can be administered in combination with an RNAi enhancer, such as RNAi-E, i.e., enoxacin.

The timing of administration of the chemical compound and the silencing element can be varied so long as the beneficial effects of the combination of these agents are achieved (i.e., modulating the silencing of a target polynucleotide of interest in a cell or in a subject). The phrase “in combination with” refers to the administration of a chemical compound with a silencing element either simultaneously, sequentially, or a combination thereof. Therefore, a cell or a subject administered a combination of the invention can receive a chemical compound and the silencing element at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the chemical compound and the silencing element are administered simultaneously, they can be administered to the cell or administered to the subject as separate pharmaceutical or cosmetic compositions, each comprising either a chemical compound or a silencing element, or they can contact the cell as a single composition or be administered to a subject as a single pharmaceutical or cosmetic composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

Further, two or more RNAi enhancers, e.g., RNAi-E and at least one compound of Formula (a)-(k) or metal chelating agents, such as iron chelating agents, which, in some embodiments, include compounds of Formula (n)-(q) as described herein, can be administered in combination, either sequentially or simultaneously, or a combination thereof. In some embodiments, when administered in combination, the two or more RNAi enhancers have a synergistic effect, as described immediately herein below. Also, in some embodiments, at least one RNAi enhancer can be administered in combination with at least one RNAi inhibitor, e.g., at least one compound of Formula (l)-(m) as described herein. When two or more RNAi enhancers, or at least one RNAi enhancer and at least one RNAi inhibitor, are administered in combination, the amount of one component, e.g., an RNAi enhancer, relative to the amount of the other component(s), e.g., an RNAi inhibitor, can vary. For example, when an RNAi enhancer is administered in combination with an RNAi inhibitor, the RNAi enhancer can be administered in a relative amount of about 99.9%, 99.5%, 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, 0.5%, and 0.1%.

As used herein, the terms “synergy,” “synergistic,” “synergistically” and derivations thereof, such as in a “synergistic effect” or a “synergistic combination” or a “synergistic composition” refer to circumstances under which the biological activity of a combination of RNAi enhancers, such as a first RNAi enhancer and at least one second RNAi enhancer, is greater than the sum of the biological activities of the respective RNAi enhancers when administered individually.

Synergy, expressed in terms of a “Synergy Index (SI),” generally can be determined by the method described by F. C. Kull, et al., Applied Microbiology 9, 538 (1961), from the ratio determined by:

Qₐ/Q_{A}+Q_{b}/Q_{B}=Synergy Index (SI)

wherein:

Q_{A} is the concentration of a component A, e.g., a first RNAi enhancer, acting alone, which produced an end point in relation to component A;

Qₐ is the concentration of component A, in a mixture, e.g., a combination of a first RNAi enhancer and a second RNAi enhancer, which produced an end point;

Q_{B} is the concentration of a component B, e.g., a second RNAi enhancer, acting alone, which produced an end point in relation to component B; and

Q_{b} is the concentration of component b, in the mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture.

Thus, a “synergistic combination” has an activity higher that what can be expected based on the observed activities of the individual components when used alone. Further, a “synergistically effective amount” of a component refers to the amount of the component necessary to elicit a synergistic effect in, for example, another RNAi enhancer present in the composition. Thus, in such embodiments, the term “synergist,” and derivations thereof, refer to a substance that enhances the activity of an RNAi enhancer.

An “effective amount” of an active agent refers to the amount of the active agent sufficient to elicit a desired biological response. As will be appreciated by one of ordinary skill in the art, the absolute amount of a particular agent that is effective can vary depending on such factors as the desired biological endpoint, the agent to be delivered, the target cell or tissue, and the like. One of ordinary skill in the art will further understand that an effective amount can be administered in a single dose, or can be achieved by administration of multiple doses.

An entity such as a gene or an expression product thereof, is considered “endogenous” to a cell if it is naturally present within the cell in the absence of modification of the cell, or an ancestor of the cell, by the hand of man. It will be appreciated that the amount of an endogenous RNA (and thus of a protein encoded by the RNA) present within a cell can be increased above its naturally occurring level by introducing a template for transcription of the RNA, operably linked to appropriate regulatory elements, into the cell. As applied to genes, RNAs, proteins, and the like, the term endogenous is generally understood to refer to genes, RNAs, proteins, and the like, as they naturally exist within a cell, unless otherwise indicated.

As used herein, the term “intracellular” or “intracellularly” has its ordinary meaning as understood in the art. In general, the space inside of a cell, which is encircled by a membrane, is defined as “intracellular” space. Similarly, as used herein, the term “extracellular” or “extracellualary” has its ordinary meaning as understood in the art. In general, the space outside of the cell membrane is defined as “extracellular” space.

As used herein, the term “gene” has its meaning as understood in the art. In general, a gene is taken to include gene regulatory sequences (e.g., promoters, enhancers, and the like) and/or intron sequences, in addition to coding sequences (open reading frames). It will further be appreciated that definitions of “gene” include references to nucleic acids that do not encode proteins but rather encode functional RNA molecules, or precursors thereof, such as microRNA or siRNA precursors, tRNAs, and the like.

The term “gene knock-down” generally refers to the use of a reagent to decrease the level of a polynucleotide of interest. As discussed in further detail elsewhere herein, polynucleotides comprising RNAi silencing elements can be used in such knock-down methods. For example, one reagent-based gene knock-down method employs siRNA or miRNAs as silencing elements. Gene knock-down by RNAi is a research tool that can be used for the analysis of gene function and for target identification and target validation.

A “gene product” or “expression product” is, in general, an RNA transcribed from the gene (e.g., either pre- or post-processing) or a polypeptide encoded by an RNA transcribed from the gene (e.g., either pre- or post-modification).

The term “hybridize” as used herein refers to the interaction between two complementary nucleic acid sequences in which the two sequences remain associated with one another under appropriate conditions.

As used herein, the term “isolated” means separated from at least some of the components with which it is usually associated in nature; prepared or purified by a process that involves the hand of man; not occurring in nature; and/or not present as an integral part of an organism.

The terms “nucleic acid,” “polynucleotide,” or “oligonucleotide” generally are used herein in their art-accepted manners to refer to a polymer of nucleotides. As used herein, an oligonucleotide is typically less than 100 nucleotides in length. Naturally occurring nucleic acids include deoxyribonucleic acid (DNA) and ribonucleic acid (RNA). The polynucleotide or oligonucleotide may include natural nucleosides (e.g., adenosine, thymidine, guanosine, cytidine, uridine, deoxyadenosine, deoxythymidine, deoxyguanosine, and deoxycytidine), or synthetic nucleosides, such as, nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), and/or nucleosides comprising chemically or biologically modified bases, (e.g., methylated bases), intercalated bases, and/or modified sugars (e.g., 2′-fluororibose, ribose, 2′-deoxyribose, arabinose, and hexose). The phosphate groups in a polynucleotide or oligonucleotide are typically considered to form the internucleoside backbone of the polymer. In naturally occurring nucleic acids (e.g., DNA or RNA), the backbone linkage is via a 3′ to 5′ phosphodiester bond. Polynucleotides and oligonucleotides containing modified backbones or non-naturally occurring internucleoside linkages, however, also can be used in the presently disclosed subject matter. Such modified backbones include backbones that have a phosphorus atom in the backbone and others that do not have a phosphorus atom in the backbone. Examples of modified linkages include, but are not limited to, phosphorothioate and 5′-N-phosphoramidite linkages. Polynucleotides and oligonucleotides need not be uniformly modified along the entire length of the molecule. For example, different nucleotide modifications, different backbone structures, and the like, may exist at various positions in the polynucleotide or oligonucleotide. Any of the polynucleotides described herein may utilize these modifications.

As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

The term “subject” refers to an organism to which the presently disclosed compounds and/or pharmaceutical or cosmetic compositions can be administered. In specific embodiments, a subject is a mammal. In other embodiments, a subject is a primate, a human, a domestic animal or an agricultural animal A cell can also be employed in the methods and compositions of the invention. Any cell can be used; however, in specific embodiments, the cell is from a mammal, a primate, a human, a domestic animal or an agricultural animal. Such host cells include cultured cells (in vitro), explants and primary cultures (in vitro and ex vivo) and cells in vivo.

As used herein, the term “treating” generally can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed active agents can be administered prophylactically to prevent or reduce the incidence or recurrence of the disease, disorder, or condition. The presently disclosed active agents also can be used for cosmetic applications, as well.

As used herein, the term “modulator” means a reagent that can influence, either enhance or inhibit, the activity of another reagent or element, e.g., a silencing element, when administered in combination a cell.

### II. Modulating Levels of Target Polynucleotides

As disclosed hereinabove, RNA interference (RNAi) is a well-conserved mechanism that uses small noncoding RNAs to silence gene expression post-transcriptionally. See Hannon (2002) Nature 418(6894): 244-51; Bartel (2004) Cell 116(2): 281-97. The endogenous small RNAs can shape diverse cellular pathways, including chromosome architecture, development, growth control, apoptosis, and stem cell maintenance. See Zamore and Haley (2005) Science 309(5740): 1519-24. Because small double-stranded RNAs, called small interfering RNAs (siRNAs), induce sequence-specific mRNA degradation when introduced into cells, RNAi has become an attractive tool for functional genomics and a potential class of molecules for human therapeutics. See Dykxhoorn and Lieberman (2006) Cell 126(2): 231-5. Although major components in the RNAi pathway have been identified, regulatory mechanisms for this pathway remain largely unknown.

The presently disclosed subject matter demonstrates that the RNAi pathway can be modulated intracellularly by small molecules. More particularly, the presently disclosed subject matter provides a novel cell-based assay to monitor the activity of the RNAi pathway and identify the RNAi pathway modulators. As disclosed herein, this cell-based assay can be used to screen a chemical library of diversified compounds to identify one or more small molecules that enhance siRNA-mediated mRNA degradation. One such small molecule, enoxacin, a second generation quinolone, is referred to herein as “RNAi-E.” In addition to RNAi-E, the presently disclosed subject matter demonstrates that other small molecules, e.g., compounds of Formula (a)-(k), when administered alone or in combination with RNAi-E, modulate the RNAi pathway.

In some embodiments, the presently disclosed subject matter demonstrates that in cells, compounds of Formula (a)-(k) or metal chelating agents, such as iron chelating agents, which, in some embodiments, include compounds of Formula (n)-(q), alone or in combination with RNAi-E, enhance the efficiency of target gene knock-down mediated by either short hairpin RNA or siRNA duplexes.

Thus, in some embodiments, the presently disclosed methods and compositions provide small molecules which modulate RNA interference in a cell and when administered in combination with at least one silencing element are employed to reduce the level of expression of a target polynucleotide of interest. As used herein, a “target sequence” comprises any sequence that one desires to decrease the level of expression. By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control which is not exposed to the silencing element. In particular embodiments, reducing the polynucleotide level and/or the polypeptide level of the target sequence according to the presently disclosed subject matter results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control. Methods to assay for the level of the RNA transcript, the level of the encoded polypeptide, or the activity of the polynucleotide or polypeptide are discussed elsewhere herein.

In other embodiments, the chemical compound decreases the activity of a silencing element. By “decrease” the activity of a silencing element is intended that the ability of the silencing element to decrease expression of a target polynucleotide is decreased by any statistically significant amount including a decrease of at least 5%, 10%, 20%, 30%, 40%, 50%, 60%, 70%, 80%, 90% or 100%.

A. Silencing Elements

As used herein, the term “silencing element” refers to a polynucleotide comprising or encoding an interfering RNA that is capable of reducing or eliminating the level of expression of a target polynucleotide or the polypeptide encoded thereby. The term “interfering RNA” or “RNAi” refers to any RNA molecule which can enter an RNAi pathway and thereby reduce the level of a target polynucleotide of interest or reduce the level of expression of a polynucleotide of interest. RNAi is distinct from so-called “antisense” mechanisms that typically involve inhibition of a target transcript by a single-stranded oligonucleotide through RNase H mediated pathway. See Crooke (ed.) (2001) “Antisense Drug Technology: Principles, Strategies, and Applications,” 1st ed., (Marcel Dekker; ISBN: 0824705661).

Thus, a silencing element can comprise the interfering RNA, a precursor to the interfering RNA, a template for the transcription of an interfering RNA or a template for the transcription of a precursor interfering RNA, wherein the precursor is processed within the cell to produce an interfering RNA. Thus, for example, a dsRNA silencing element includes a dsRNA molecule, a transcript or polyribonucleotide capable of forming a dsRNA, more than one transcript or polyribonucleotide capable of forming a dsRNA, a DNA encoding dsRNA molecule, or a DNA encoding one strand of a dsRNA molecule. When the silencing element comprises a DNA molecule encoding an interfering RNA, it is recognized that the DNA can be transiently expressed in a cell or stably incorporated into the genome of the cell. Such methods are discussed in further detail elsewhere herein.

The silencing element can reduce or eliminate the expression level of a target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, e.g., Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional interfering RNA that are capable of reducing or eliminating the level of a sequence of interest are disclosed elsewhere herein.

Any region of the target polynucleotide can be used to design a domain of the silencing element that shares sufficient sequence identity to allow for the silencing element to decrease the level of the target polynucleotide. For instance, the silencing element can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof.

The ability of a silencing element to reduce the level of the target polynucleotide may be assessed directly by measuring the amount of the target transcript using, for example, Northern blots, nuclease protection assays, reverse transcription (RT)-PCR, real-time RT-PCR, microarray analysis, and the like. Alternatively, the ability of the silencing element to reduce the level of the target polynucleotide may be measured directly using a variety of affinity-based approaches (e.g., using a ligand or antibody that specifically binds to the target polypeptide) including, but not limited to, Western blots, immunoassays, ELISA, flow cytometry, protein microarrays, and the like. In still other methods, the ability of the silencing element to reduce the level of the target polynucleotide can be assessed indirectly, e.g., by measuring a functional activity of the polypeptide encoded by the transcript or by measuring a signal produced by the polypeptide encoded by the transcript.

Representative types of silencing elements are discussed in further detail below.

i. Double Stranded RNA Silencing Elements

In one embodiment, the silencing element comprises or encodes a double stranded RNA molecule. As used herein, a “double stranded RNA” or “dsRNA” refers to a polyribonucleotide structure formed either by a single self-complementary RNA molecule or a polyribonucleotide structure formed by the expression of least two distinct RNA strands. Accordingly, as used herein, the term “dsRNA” is meant to encompass other terms used to describe nucleic acid molecules that are capable of mediating RNA interference or gene silencing, including, for example, small RNA (sRNA), short-interfering RNA (siRNA), double-stranded RNA (dsRNA), micro-RNA (miRNA), hairpin RNA, short hairpin RNA (shRNA), and others. See, e.g., Meister and Tuschl (2004) Nature 431:343-349 and Bonetta et al. (2004) Nature Methods 1:79-86.

In specific embodiments, at least one strand of the duplex or double-stranded region of the dsRNA shares sufficient sequence identity or sequence complementarity to the target polynucleotide to allow for the dsRNA to reduce the level of expression of the target sequence. As used herein, the strand that is complementary to the target polynucleotide is the “antisense strand,” and the strand homologous to the target polynucleotide is the “sense strand.”

In one embodiment, the dsRNA comprises a hairpin RNA. A hairpin RNA comprises an RNA molecule that is capable of folding back onto itself to form a double stranded structure. Multiple structures can be employed as hairpin elements. For example, the hairpin RNA molecule that hybridizes with itself to form a hairpin structure can comprises a single-stranded loop region and a base-paired stem. The base-paired stem region can comprise a sense sequence corresponding to all or part of the target polynucleotide and further comprises an antisense sequence that is fully or partially complementary to the sense sequence. Thus, the base-paired stem region of the silencing element can determine the specificity of the silencing. See, e.g., Chuang and Meyerowitz (2000) Proc. Natl. Acad. Sci. USA 97:4985-4990, herein incorporated by reference. A transient assay for the efficiency of hpRNA constructs to silence gene expression in vivo has been described by Panstruga et al. (2003) Mol. Biol. Rep. 30:135-140, herein incorporated by reference.

ii. siRNA Silencing Elements

A “short interfering RNA” or “siRNA” comprises an RNA duplex (double-stranded region) and can further comprises one or two single-stranded overhangs, e.g., 3′ or 5′ overhangs. The duplex can be approximately 19 base pairs (bp) long, although lengths between 17 and 29 nucleotides, including 17, 18, 19, 20, 21, 22, 23, 24, 25, 26, 27, 28, and 29 nucleotides, can be used. An siRNA can be formed from two RNA molecules that hybridize together or can alternatively be generated from a single RNA molecule that includes a self-hybridizing portion. The duplex portion of an siRNA can include one or more bulges containing one or more unpaired and/or mismatched nucleotides in one or both strands of the duplex or can contain one or more noncomplementary nucleotide pairs. One strand of an siRNA (referred to herein as the antisense strand) includes a portion that hybridizes with a target transcript. In certain embodiments, one strand of the siRNA (the antisense strand) is precisely complementary with a region of the target transcript over at least about 17 nucleotides, 18 nucleotides, 19 nucleotides, 20 nucleotides or more meaning that the siRNA antisense strand hybridizes to the target transcript without a single mismatch (i.e., without a single noncomplementary base pair) over that length. In other embodiments, one or more mismatches between the siRNA antisense strand and the targeted portion of the target transcript can exist. In embodiments in which perfect complementarity is not achieved, any mismatches between the siRNA antisense strand and the target transcript can be located at or near 3′ end of the siRNA antisense strand. For example, in certain embodiments, nucleotides 1-9, 2-9, 2-10, and/or 1-10 of the antisense strand are perfectly complementary to the target.

Considerations for design of effective siRNA molecules are discussed in McManus et al. (2002) Nature Reviews Genetics 3: 737-747 and in Dykxhoorn et al. (2003) Nature Reviews Molecular Cell Biology 4: 457-467. Such considerations include the base composition of the siRNA, the position of the portion of the target transcript that is complementary to the antisense strand of the siRNA relative to the 5′ and 3′ ends of the transcript, and the like. A variety of computer programs also are available to assist with selection of siRNA sequences, e.g., from Ambion (web site having URL www.ambion.com), at web site having URL www.sinc.sunysb.edu/Stu/shilin/rnai.html. Additional design considerations that also can be employed are described in Semizarov et al. Proc. Natl. Acad. Sci. 100: 6347-6352.

iii. Short Hairpin RNA Silencing Elements

The term “short hairpin RNA” or “shRNA” refers to an RNA molecule comprising at least two complementary portions hybridized or capable of hybridizing to form a double-stranded (duplex) structure sufficiently long to mediate RNAi (generally between approximately 17 and 29 nucleotides in length, including 17, 18, 19, 20, 21, 22, 23, 24, 25, 26, 27, 28, and 29 nucleotides in length, and in some embodiments, typically at least 19 base pairs in length), and at least one single-stranded portion, typically between approximately 1 and 20 or 1 to 10 nucleotides in length that forms a loop connecting the two nucleotides that form the base pair at one end of the duplex portion. The duplex portion can, but does not require, one or more bulges consisting of one or more unpaired nucleotides. In specific embodiments, the shRNAs comprise a 3′ overhang. Thus, shRNAs are precursors of siRNAs and are, in general, similarly capable of inhibiting expression of a target transcript.

In particular, RNA molecules having a hairpin (stem-loop) structure can be processed intracellularly by Dicer to yield an siRNA structure referred to as short hairpin RNAs (shRNAs), which contain two complementary regions that hybridize to one another (self-hybridize) to form a double-stranded (duplex) region referred to as a stem, a single-stranded loop connecting the nucleotides that form the base pair at one end of the duplex, and optionally an overhang, e.g., a 3′ overhang. The stem can comprise about 19, 20, or 21 bp long, though shorter and longer stems (e.g., up to about 29 nt) also can be used. The loop can comprise about 1-20, including 1, 2, 3, 4, 5, 6, 7, 8, 9, 10, 11, 12, 13, 14, 15, 16, 17, 18, 19, 20 nt, about 4-10, or about 6-9 nt. The overhang, if present, can comprise approximately 1-20 nt or approximately 2-10 nt. The loop can be located at either the 5′ or 3′ end of the region that is complementary to the target transcript whose inhibition is desired (i.e., the antisense portion of the shRNA).

Although shRNAs contain a single RNA molecule that self-hybridizes, it will be appreciated that the resulting duplex structure can be considered to comprise sense and antisense strands or portions relative to the target mRNA and can thus be considered to be double-stranded. It will therefore be convenient herein to refer to sense and antisense strands, or sense and antisense portions, of an shRNA, where the antisense strand or portion is that segment of the molecule that forms or is capable of forming a duplex with and is complementary to the targeted portion of the target polynucleotide, and the sense strand or portion is that segment of the molecule that forms or is capable of forming a duplex with the antisense strand or portion and is substantially identical in sequence to the targeted portion of the target transcript. In general, considerations for selection of the sequence of the antisense strand of an shRNA molecule are similar to those for selection of the sequence of the antisense strand of an siRNA molecule that targets the same transcript.

iv. MicroRNA Silencing Elements

In one embodiment, the silencing element comprises an miRNA. MicroRNAs” or “miRNAs” are regulatory agents comprising about 19 ribonucleotides which are highly efficient at inhibiting the expression of target polynucleotides. See, e.g., Saetrom et al. (2006) Oligonucleotides 16:115-144, Wang et al. (2006) Mol. Cell. 22:553-60, Davis et al. (2006) Nucleic Acid Research 34:2294-304, Pasquinelli (2006) Dev. Cell 10:419-24, all of which are herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

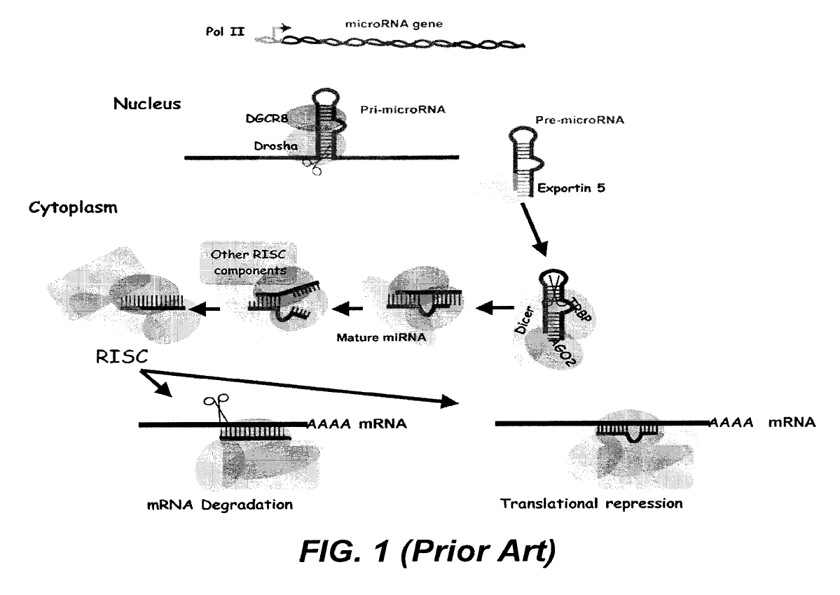

Referring now to FIG. 1, a schematic diagram of the miRNA suppression pathway is shown. It is recognized that various forms of an miRNA can be transcribed including, for example, the primary transcript (termed the “pri-miRNA”) which is processed through various nucleolytic steps to a shorter precursor miRNA (termed the “pre-miRNA”); the pre-miRNA; or the final (mature) miRNA is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (McManus et al. (2002) RNA 8:842-50). In specific embodiments, 2-8 nucleotides of the miRNA are perfectly complementary to the target. A large number of endogenous human miRNAs have been identified. For structures of a number of endogenous miRNA precursors from various organisms, see Lagos-Quintana et al. (2003) RNA 9(2):175-9; see also Bartel (2004) Cell 116:281-297.

A miRNA or miRNA precursor can share at least about 80%, 85%, 90%, 91%.

92%, 93%, 94%, 95%, 96%, 97%, 98%, 99%, or 100% sequence complementarity with the target transcript for a stretch of at least about 15, 16, 17, 18, 19, 20, 21, 22, 23, 24, 25, 26, 27, 28, 29, or 30 nucleotides. In specific embodiments, the region of precise sequence complementarity is interrupted by a bulge. See Ruvkun (2001) Science 294: 797-799, Zeng et al. (2002) Molecular Cell 9:1-20, and Mourelatos et al. (2002) Genes Dev 16:720-728.

B. Preparing Silencing Elements

Those of ordinary skill in the art will readily appreciate that a silencing element can be prepared according to any available technique including, but not limited to, chemical synthesis, enzymatic or chemical cleavage in vivo or in vitro, template transcription in vivo or in vitro, or combinations of the foregoing.

i. Recombinant Expression Vectors and Host Cells

As discussed above, the silencing elements employed in the methods and compositions of the invention can comprise a silencing element. In specific embodiments, the silencing element comprises a DNA molecule which when transcribed produces an interfering RNA or a precursor thereof. In such embodiments, the DNA molecule encoding the silencing element is found in an expression cassette.

The expression cassette comprises one or more regulatory sequences, selected on the basis of the cells to be used for expression, operably linked to a polynucleotide encoding the silencing element. “Operably linked” is intended to mean that the nucleotide sequence of interest (i.e., a DNA encoding a silencing element) is linked to the regulatory sequence(s) in a manner that allows for expression of the nucleotide sequence (e.g., in an in vitro transcription/translation system or in a cell when the expression cassette or vector is introduced into a cell). “Regulatory sequences” include promoters, enhancers, and other expression control elements (e.g., polyadenylation signals). See, e.g., Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression cassette can depend on such factors as the choice of the host cell to be transformed, the level of expression of the silencing element desired, and the like. Such expression cassettes typically include one or more appropriately positioned sites for restriction enzymes, to facilitate introduction of the nucleic acid into a vector.

It will further be appreciated that appropriate promoter and/or regulatory elements can readily be selected to allow expression of the relevant transcription units/silencing elements in the cell of interest. In certain embodiments, the promoter utilized to direct intracellular expression of a silencing element is a promoter for RNA polymerase III (Pol III). References discussing various Pol III promoters, include, for example, Yu et al. (2002) Proc. Natl. Acad. Sci. 99(9), 6047-6052; Sui et al. (2002) Proc. Natl. Acad. Sci. 99(8), 5515-5520; Paddison et al. (2002) Genes and Dev. 16, 948-958; Brummelkamp et al. (2002) Science 296, 550-553; Miyagashi (2002) Biotech. 20, 497-500; Paul et al. (2002) Nat. Biotech. 20, 505-508; Tuschl et al. (2002) Nat. Biotech. 20, 446-448. According to other embodiments, a promoter for RNA polymerase I, e.g., a tRNA promoter, can be used. See McCown et al. (2003) Virology 313(2):514-24; Kawasaki (2003) Nucleic Acids Res. 31 (2):700-7.

The regulatory sequences can also be provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See Goeddel (1990) in Gene Expression Technology: Methods in Enzymology 185 (Academic Press, San Diego, Calif.).

In vitro transcription may be performed using a variety of available systems including the T7, SP6, and T3 promoter/polymerase systems (e.g., those available commercially from Promega, Clontech, New England Biolabs, and the like). Vectors including the T7, SP6, or T3 promoter are well known in the art and can readily be modified to direct transcription of silencing elements. When silencing elements are synthesized in vitro the strands may be allowed to hybridize before introducing into a cell or before administration to a subject. As noted above, silencing elements can be delivered or introduced into a cell as a single RNA molecule including self-complementary portions (e.g., an shRNA that can be processed intracellularly to yield an siRNA), or as two strands hybridized to one another. In other embodiments, the silencing elements employed are transcribed in vivo. As discussed elsewhere herein, regardless of if the silencing element is transcribed in vivo or in vitro, in either scenario, a primary transcript can be produced which is then be processed (e.g., by one or more cellular enzymes) to generate the interfering RNA that accomplishes gene inhibition.

Such expression cassettes can be contained in a vector which allow for the introduction of the expression cassette into a cell. In specific embodiments, the vector allows for autonomous replication of the expression cassette in a cell or may be integrated into the genome of a cell. Such vectors are replicated along with the host genome (e.g., nonepisomal mammalian vectors). In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses).

Accordingly, the interfering RNA may be generated by transcription from a promoter, either in vitro or in vivo. For instance, a construct may be provided containing two separate transcribable regions, each of which generates a 21 nt transcript containing a 19 nt region complementary with the other. Alternatively, a single construct may be utilized that contains opposing promoters and terminators positioned so that two different transcripts, each of which is at least partly complementary to the other, are generated. Alternatively, an RNA-inducing agent may be generated as a single transcript, for example by transcription of a single transcription unit encoding self complementary regions. A template is employed that includes first and second complementary regions, and optionally includes a loop region connecting the portions. Such a template may be utilized for in vitro transcription or in vivo transcription, with appropriate selection of promoter and, optionally, other regulatory elements, e.g., a terminator.

ii. Administering a Silencing Element to a Cell

“Administering” a silencing element to a cell comprises any method that allows for the introduction of the polynucleotide into the cell including any conventional transformation or transfection techniques. Exemplary art-recognized techniques for introducing foreign polynucleotides into a host cell, including calcium phosphate or calcium chloride co-precipitation, DEAE-dextran-mediated transfection, lipofection, particle gun, or electroporation and viral vectors. Suitable methods for transforming or transfecting host cells can be found in U.S. Pat. No. 5,049,386, U.S. Pat. No. 4,946,787; and U.S. Pat. No. 4,897,355, Sambrook et al. (1989) Molecular Cloning: A Laboratory Manual (2d ed., Cold Spring Harbor Laboratory Press, Plainview, N.Y.) and other standard molecular biology laboratory manuals. One of skill will recognize that depending on the method by which a polynucleotide is introduced into a cell, the silencing element can be stably incorporated into the genome of the cell, replicated on an autonomous vector or plasmid, or present transiently in the cell.

Viral vector delivery systems include DNA and RNA viruses, which have either episomal or integrated genomes after delivery to the cell. For a review of viral vector procedures, see Anderson (1992) Science 256:808-813; Haddada et al. (1995) Current Topics in Microbiology and Immunology Doerfler and Bohm (eds); and Yu et al. (1994) Gene Therapy 1:13-26.

Viral vectors can be administered directly to patients (in vivo) or they can be used to treat cells in vitro and the modified cells are administered to patients (ex vivo). Conventional viral based systems for the delivery of silencing elements could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene.

Viral vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient, followed by reimplantation of the cells into a patient, usually after selection for cells which have incorporated the vector.

In specific embodiments, the silencing element administered to a cell is heterologous to the cell. As used herein, “heterologous” in reference to a sequence is a sequence that originates from a foreign species, or, if from the same species, is substantially modified from its native form in composition and/or genomic locus by deliberate human intervention. For example, a heterologous silencing element is from a species different from the species of the host cell, or, if from the same/analogous species, the silencing element is substantially modified from its native form in composition, length, and genomic locus. The silencing element could be a portion of the original form.

III. Methods for Screening Compounds that Modulate the Activity of Silencing Elements

Methods and compositions are provided that increase or decrease the efficacy of RNAi in a cell or an organism, e.g., to control the extent to which a silencing element, such as an siRNA, hairpin RNA, shRNA, miRNA, and the like, present in the cell or organism is able to inhibit expression of a target polynucleotide. In specific embodiments, methods and compositions are provided that enhance the activity of a silencing element and thereby enhance the ability of a silencing element to reduce the level of a target polynucleotide.

In principle, interfering RNA libraries can be used to perform effective genome-scale functional genetic screens in mammalian cells or whole animals to identify molecules that modulate the activity of silencing elements. Accordingly, in some embodiments, the presently disclosed subject matter provides a cell-based reporter system to identify chemical compounds that could modulate the activity of an interfering RNA.

In this system, a host cell expressing a heterologous reporter gene is used to screen compounds of interest for their ability to modulate (e.g., enhance or inhibit) the effects of a heterologous silencing element by introducing a heterologous silencing element directed against the reporter gene expressed by the host cell and exposing the cell to the compound of interest. A compound that leads to an increase in expression of the reporter gene in this system is considered an inhibitor of RNAi, whereas a compound that leads to a decrease in expression of the reporter gene in this system is considered an enhancer of RNAi activity. In this context, an “increase” or a “decrease” in the expression of the reporter gene in the presence of the reporter gene-specific RNAi is relative to the expression of the reporter gene in the presence of the reporter gene-specific RNAi without exposure to the compound of interest.

The reporter gene used in the methods of the presently disclosed subject matter is one whose level of expression can be monitored. In specific embodiments, the level of expression of the reporter gene is visible to the aided or unaided eye. By “aided” is intended the use of a device to facilitate visualization (i.e., light of any visible wavelength, a microscope or other magnifying instrument, computer hardware or software, or other device capable of detecting, quantitating and/or displaying the visible medium corresponding to the expression level of the reporter gene, such as a fluorometer, luminometer or densitometer). In some embodiments, expression is correlated directly or relatively (e.g., precisely or semi-precisely) with the visible medium (e.g., the amount of light, color, density, and the like, resulting from expression of the reporter gene). The visible medium can be measured manually or automatically by, for example, counting the number of cells in a designated field of view that are colored, fluorescent, luminescent, pixilated, or otherwise visible to the aided or unaided eye, or by quantitating the amount of visible fluorescence, luminescence, color, density, and the like, in all or part of a designated field of view using an instrument or device for performing such a function.

Examples of such reporter genes include, but are not limited to, those that encode green fluorescent protein (GFP), luciferase, or beta-galactosidase. The reporter gene can be introduced into the host cell using methods routine to those of skill in the art. In some embodiments, the DNA encoding the reporter gene is found in an expression cassette as described elsewhere herein. Such expression cassettes can be contained in a vector which allows for the introduction of the expression cassette comprising the reporter gene into a host cell. In specific embodiments, a viral vector, particularly a lentiviral vector, is used to introduce the reporter gene into a host cell. A host cell used in the presently disclosed methods allows for the introduction and expression of heterologous nucleic acids and can be maintained in a liquid, solid or semi-solid cell culture media. Any appropriate host cell can be used. In one embodiment, the human 293 cell line stably expressing the reporter gene GFP is used. One of ordinary skill in the art to which the presently disclosed subject matter pertains, would recognize upon review of the presently disclosed subject matter that any cell line, from insect to human cells, could be used in the presently disclosed screening methods. Exemplary cell lines include, but are not limited to, S2 cells, NKH3T3, NS20Y, HeLa, and HepG2 cells.

To screen for novel compounds that modulate the activity of interfering RNA, cells should be selected that express a moderate level of the reporter gene (i.e., GFP) in the presence of the interfering RNA. In specific embodiments, the moderate level of expression comprises a level of expression that allows one to detect a statistically significant increase or decrease in the level of the expression of the reporter gene. By moderate level is intended between 10% and 70%, between 15% and 60%, between 20% and 50%, or between 25% and 45% of the level of expression of the reporter gene in the absence of interfering RNA. Further, an appropriate clone is selected after the addition of siRNAs.

Thus, in some embodiments, the presently disclosed subject matter provides a method for screening a compound of interest for the ability to modulate the activity of a heterologous silencing element, the method comprising: (a) providing a host cell that stably expresses a reporter gene, wherein said host cell further comprises at least one heterologous silencing element capable of inhibiting the expression of the reporter gene; (b) administering to the cell a compound of interest; and (c) measuring the expression of the reporter gene. In some embodiments, the silencing element comprises an siRNA, an miRNA, a double stranded RNA, or a hairpin RNA. In some embodiments, the reporter gene encodes green fluorescent protein.

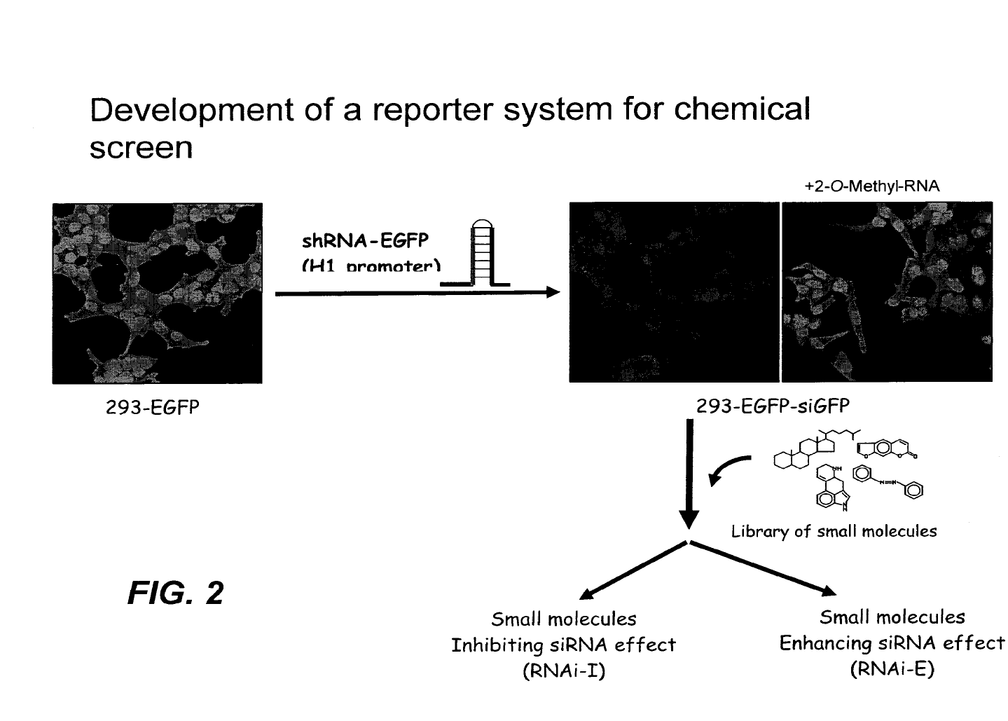

FIG. 2 discloses one embodiment of the cell-based reporter system of the invention. In this example, a human 293-cell line stably expressing a reporter gene, GFP, was used. This cell line was further transfected with a construct that expresses siRNA hairpin against GFP, which can decrease the level of GFP expression. Individual clones were isolated with greatly (although not completely) reduced GFP expression.

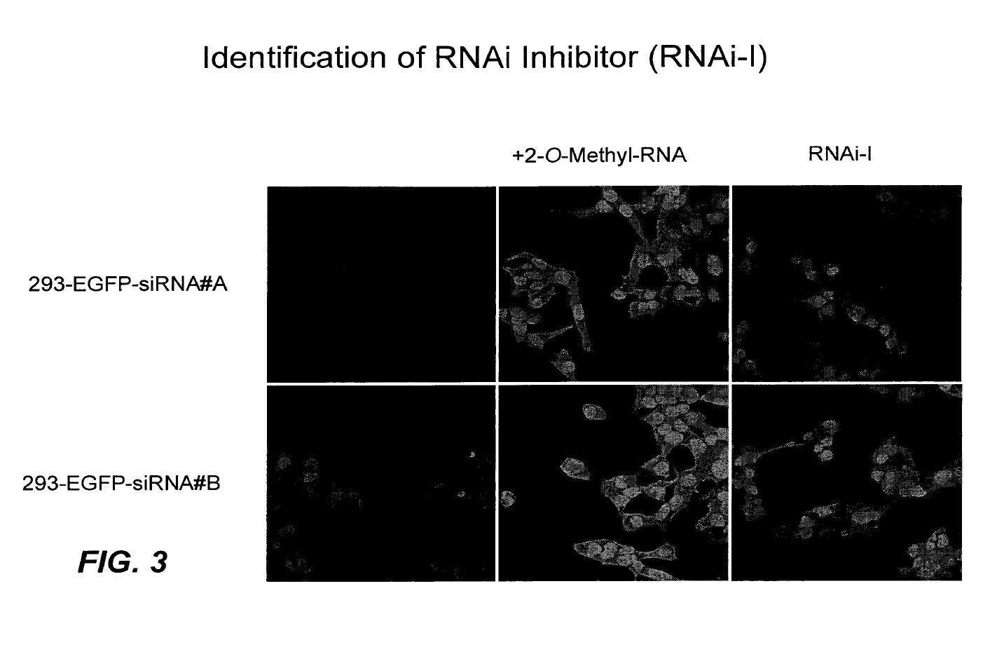

Referring now to FIG. 3, to verify that the decrease of GFP expression in the presently disclosed reporter system is due to GFP siRNA, 2-O-methyl RNA, which has been shown to block the siRNA effect previously, was transfected against GFP siRNA into those cells. As shown in FIG. 3, second panel, the GFP expression increased.

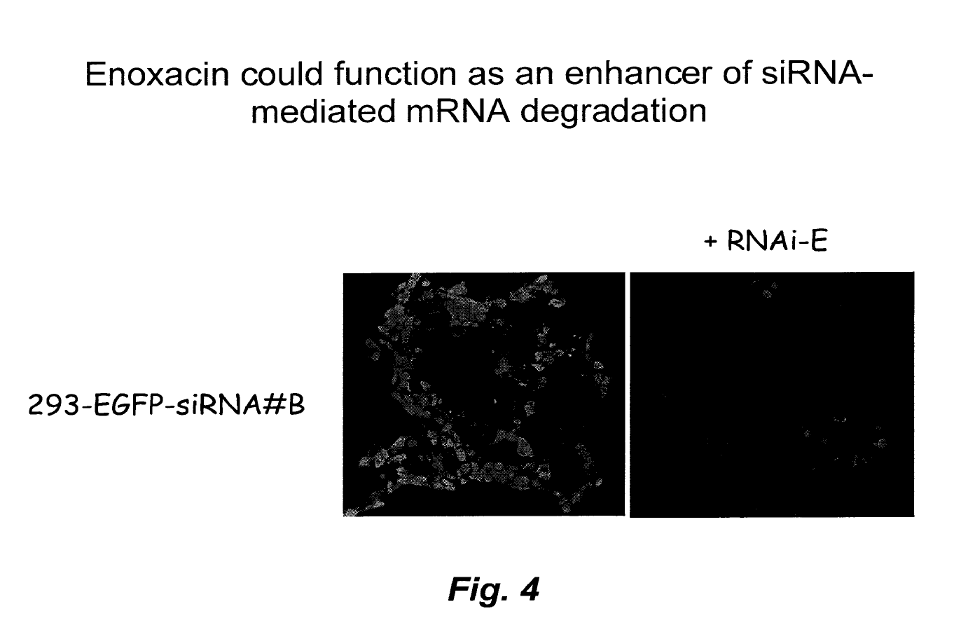

Using the above-identified clones, a screen was performed using The Spectrum Collection (MicroSource Discovery Systems, Inc., Gaylordsville, Conn., United States of America), which contains 2000 biologically active and structurally diverse compounds from libraries of known drugs, experimental bioactives, and pure natural products. For example, in one embodiment, a pilot screen of this library identified one inhibitor and one enhancer of siRNA-mediated mRNA degradation. Referring once again to FIG. 3, the compound “trimethobenzamide” was found to inhibit siRNA-mediated mRNA degradation and gene knock-down (FIG. 3, third panel). Referring now to FIG. 4, the compound “enoxacin” was found to enhance siRNA-mediated mRNA degradation and gene knock-down.

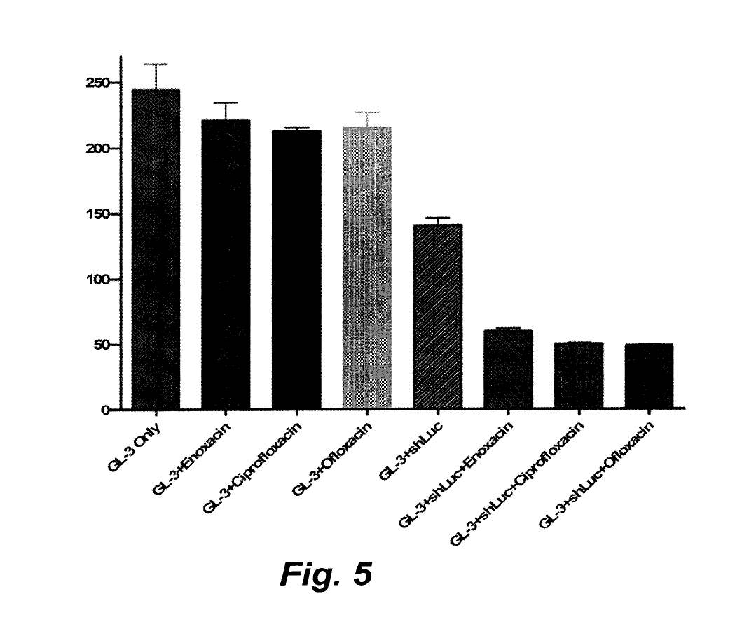

Enoxacin is a quinolone-type antibiotic that has been approved for clinical use by the FDA for the treatment of certain infections caused by bacteria, such as gonorrhea and urinary tract infections. Referring now to FIG. 5, other quinolones, e.g., ciprofloxacin and ofloxacin, also enhance siRNA-mediated mRNA degradation. Such enhancers can be directly applied to RNAi technology to increase the efficiency of knocking down the endogenous genes and can be directly applied to different experimental systems to improve the efficiency of decreasing the level of a polynucleotide of interest.

Further, in some embodiments, additional screens using the same library can be performed to identify other modulators of the siRNA and the miRNA pathways. In some embodiments, instead of examining the GFP signal visually under a microscope, the GFP signal can be quantified using an automatic plate reader. In such embodiments, two additional rounds of screening can be performed. In the first round of screening, the clones can be treated with compounds from a library, e.g., The Spectrum Collection, alone and the GFP level can be quantified after a period of time, e.g., 48 hours. In the second round of screening, the clones can be treated with compounds from a library, e.g., The Spectrum Collection, in the presence of 50 μM RNAi-E, i.e., enoxacin. This assay enables compounds that can modulate the activity of RNAi-E to be identified.

Thus, in some embodiments, the presently disclosed subject matter provides a method for screening a compound of interest for the ability to modulate the activity of a heterologous silencing element comprising: (a) providing a host cell that stably expresses a reporter gene, wherein said host cell further comprises at least one heterologous silencing element capable of inhibiting the expression of the reporter gene; (b) administering to the cell a compound of interest in the presence of an RNAi enhancer; and (c) measuring the expression of the reporter gene. In some embodiments, the RNAi enhancer comprises enoxacin, or derivatives or analogs thereof.

IV. Compounds that Modulate the Activity of a Silencing Element

In some embodiments, the presently disclosed subject matter provides a method for modulating the level of a target polynucleotide in a cell, the method comprising administering to the cell an effective amount of at least one RNAi modulating compound, wherein said cell further comprises at least one heterologous silencing element. In some embodiments, the RNAi modulating compound comprises at least one RNAi enhancing compound. In some embodiments, the RNAi modulating compound comprises at least one RNAi inhibitory compound.

A. Enhancers of RNAi

In some embodiments, the presently disclosed modulator is an RNAi enhancer, which increases the silencing element's ability to decrease the level of a target polynucleotide when inside a cell. Accordingly, in some embodiments, the presently disclosed subject matter provides a method for decreasing the level of a target polynucleotide in a cell, the method comprising administering to the cell an effective amount of a compound of Formula (a)-(k), or a derivative or analog thereof, alone or in combination with an RNAi-E, such as enoxacin, wherein said cell further comprises at least one heterologous silencing element:

(a) triprolidine, derivatives, and analogs thereof:

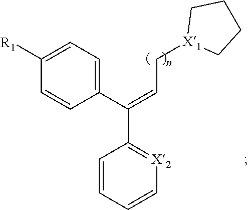

(b) dihydroptaeroxylin, derivatives, and analogs thereof:

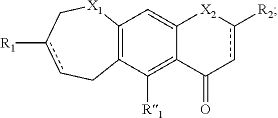

(c) fusidic acid, derivatives, and analogs thereof:

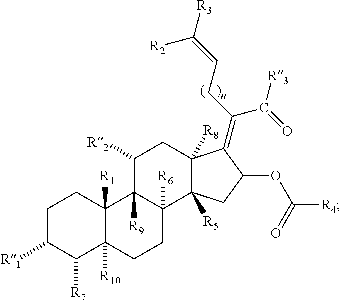

(d) fenbufen, derivatives, and analogs thereof:

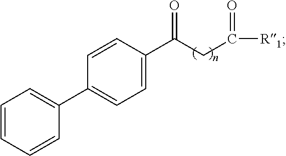

(e) 3-beta-hydroxydeoxyodihydrodeoxygedunin, derivatives, and analogs thereof:

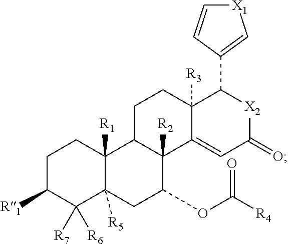

(f) deferoxamine, derivatives, and analogs thereof:

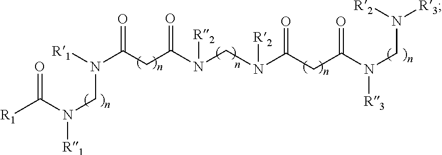

(g) thioguanine, derivatives, and analogs thereof:

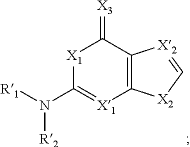

(h) 2-aminomethyl-1,4-benzodioxane, derivatives, and analogs thereof:

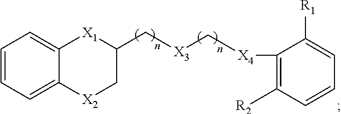

(i) 3-alpha-hydroxy-3-deoxyangolensic acid methyl ester, derivatives, and analogs thereof:

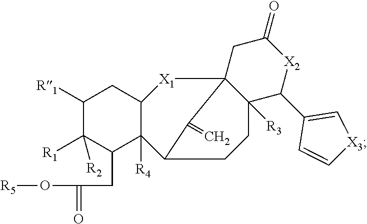

(j) lunarine, derivatives, and analogs thereof:

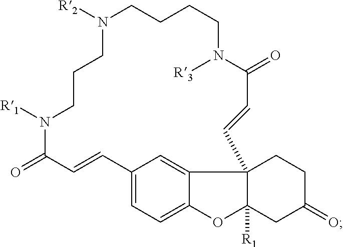

and
(k) bromocriptine, derivatives, and analogs thereof:

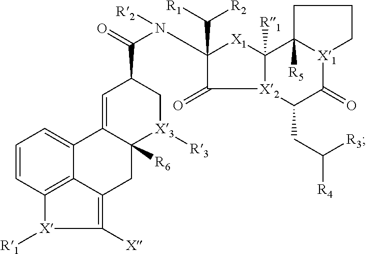

wherein:

each n is independently an integer from 1 to 20;

a dashed line in a cyclic ring structure represents a bond that can be either present or absent in the ring;

each R₁, R₂, R₃, R₄, R₅, R₆, R₇, R₈, R₉, and R₁₀ is independently selected from the group consisting of H, alkyl, substituted alkyl, cycloalkyl, substituted cycloalkyl, aryl, and substituted aryl;

each R′₁, R′₂, and R′₃ is independently selected from the group consisting of H, alkyl, substituted alkyl, cycloalkyl, substituted cycloalkyl, aryl, substituted aryl, hydroxyl, and alkoxyl;

each R″₁, R″₂, and R″₃ is independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;

each X₁, X₂, X₃, and X₄ is independently selected from the group consisting of CH₂, O, S, and NR'₄, wherein R′₄ is selected from the group consisting of H, alkyl, substituted alkyl, cycloalkyl, substituted cycloalkyl, aryl, substituted aryl, hydroxyl, and alkoxyl;

each X′₁, X′₂, and X′₃ is independently N or CH; and

each X″ is independently halogen; and

pharmaceutically and cosmetically acceptable salts thereof.

In some embodiments, the compound of Formula (a)-(k) is selected from the group consisting of triprolidine, dihydropaeroxylin, fusidic acid, fenbufen, 3-beta-hydroxydeoxodihydrodeoxygedunin, deferoxamine, thioguanin, 2-aminomethyl-1,4-benzodioxane, 3-alpha-hydroxy-3-deoxyangloensic acid methyl ester, lunarine, bromocriptine, and pharmaceutically and cosmetically acceptable salts thereof.

In some embodiments, the silencing element comprises an siRNA, an miRNA, a double stranded RNA, or a hairpin RNA. In some embodiments, the cell is in a subject.

In some embodiments, the presently disclosed subject matter provides a method for decreasing the level of a target polynucleotide in a cell, the method comprising administering to said cell a combination of an effective concentration of a polynucleotide comprising a heterologous silencing element and an effective amount of a compound of Formula (a)-(k), derivatives and analogs thereof, as defined hereinabove, alone or in combination with an RNAi-E, such as enoxacin.

In some embodiments, the polynucleotide comprising the heterologous silencing element comprises an expression cassette encoding a siRNA, a miRNA, a dsRNA, or a hairpin RNA. In some embodiments, the polynucleotide is in a viral vector. In some embodiments, the polynucleotide comprises a siRNA, a miRNA, a dsRNA, or a hairpin RNA.

In some embodiments, the compound of Formula (a)-(k), alone or in combination with an RNAi-E, such as enoxacin, and the heterologous silencing element are administered to the cell simultaneously or sequentially. In some embodiments, the cell is in a subject. In some embodiments, the cell is from a mammal.

In some embodiments, the presently disclosed subject matter provides a pharmaceutical or cosmetic composition comprising at least one of a compound of Formula (a)-(k), alone or in combination with an RNAi-E, such as enoxacin, and a pharmaceutically or cosmetically acceptable carrier and one or more polynucleotides comprising a silencing element. In some embodiments, the silencing element comprises a siRNA, a miRNA, a double stranded RNA, or a hairpin RNA.

In some embodiments, at least one compound of Formula (a)-(k) can be administered in combination with another RNAi enhancer. Thus, the presently disclosed subject matter provides, in some embodiments, a method for decreasing the level of a target polynucleotide in a cell, the method comprising administering to the cell an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is selected from the group consisting of a compound of Formula (a)-(k), and derivatives and analogs thereof.

In some embodiments, the presently disclosed subject matter provides a method for decreasing the level of a target polynucleotide in a cell, the method comprising administering to said cell a combination of an effective concentration of a polynucleotide comprising a heterologous silencing element and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is selected from the group consisting of a compound of Formula (a)-(k), and derivatives and analogs thereof.

In some embodiments, at least one of the first RNAi enhancer and the at least one second RNAi enhancer comprises enoxacin, or a derivative or analog thereof. In some embodiments, the first RNAi enhancer and the at least one second RNAi enhancer and said heterologous silencing element are administered to the cell simultaneously or sequentially.

In some embodiments, the presently disclosed subject matter provides pharmaceutical or cosmetic composition comprising at a first RNAi enhancer in combination with at least one second RNAi enhancer, wherein at least one of the first RNAi enhancer and the second RNAi enhancer is selected from the group consisting of a compound of Formula (a)-(k), and derivatives and analogs thereof, and a pharmaceutically or cosmetically acceptable carrier and one or more polynucleotides comprising a silencing element.

B. Suppressors of RNAi

In some embodiments, the presently disclosed subject matter provides a method for suppressing the activity of a silencing element. The method comprises administering to a cell a combination of an effective amount of at least one silencing element and an effective amount of at least one inhibitory compound. More particularly, in some embodiments, the presently disclosed subject matter provides a method for decreasing the activity of a silencing element in a cell, the method comprising administering to the cell an effective amount of a compound of Formula (l)-(m) or a derivative or analog thereof:

(l) colforsin, derivatives, and analogs thereof:

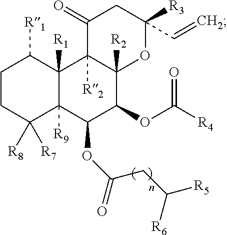

and
(m) metameconine, derivatives, and analogs thereof:

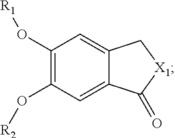

wherein:

n is an integer from 1 to 20;

each R₁, R₂, R₃, R₄, R₅, R₆, R₇, R₈, and R₉ is independently selected from the group consisting of H, alkyl, substituted alkyl, cycloalkyl, substituted cycloalkyl, aryl, and substituted aryl;

R″₁ and R″₂ are each independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;

X₁ is selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl; and pharmaceutically and cosmetically acceptable salts thereof.

In some embodiments, the compound of Formula (l)-(m) is selected from the group consisting of colforsin and metameconine and pharmaceutically and cosmetically acceptable salts thereof. In some embodiments, the cell is in a subject.

In some embodiments, at least one compound of Formula (l)-(m) is administered in combination with an RNAi enhancer, such as RNAi-E, i.e., enoxacin. In such embodiments, the at least one compound of Formula (l)-(m) inhibits the activity of RNAi-E, thereby modulating siRNA function. Such embodiments have use, for example, when siRNAs have a low knock-down efficiency and are targeted for important genes. One non-limiting application for such embodiments includes using at least one compound of Formula (l)-(m) to inhibit siRNA activity initially, and then, when desired, withdraw the at least one compound of Formula (l)-(m), thereby allowing RNAi-E to enhance the RNAi effect. The at least one compound of Formula (l)-(m) could then be applied again to suppress siRNA activity to maintain a healthy subject.

Thus, in some embodiments, the presently disclosed subject matter provides a method for modulating the activity of a silencing element in a cell, the method comprising administering to the cell an effective amount of at least one RNAi enhancer and an effective amount of at least one compound of Formula (l)-(m) or a derivative or analog thereof, to modulate the activity of said silencing element. In some embodiments, the at least one compound of Formula (l)-(m) is selected from the group consisting of colforsin and metameconine and pharmaceutically and cosmetically acceptable salts thereof. In some embodiments, the at least one RNAi enhancer is RNAi-E, e.g., enoxacin, or a derivative or analog thereof. In some embodiments, the cell is in a subject.

In some embodiments, the presently disclosed subject matter provides a pharmaceutical or cosmetic composition comprising a compound of Formula (l)-(m), alone or in combination with an RNAi enhancer, a pharmaceutically or cosmetically acceptable carrier, and one or more polynucleotides comprising a silencing element, which when administered to a subject, decreases the level of a target polynucleotide.

In some embodiments, the presently disclosed RNAi inhibitors can be used to control the timing of siRNA-mediated mRNA degradation. Further, it has been shown that overexpression of miRNAs can lead to tumorgenesis. Thus, because an RNAi-I could modulate miRNA-mediated translational regulation, the presently disclosed RNAi inhibitors also can be used to alter the activity of endogenous miRNAs.

C. Metal Chelators for Enhancing Endogenous RNAi Activity

i. General Considerations

Indispensable for life, iron (Fe) is involved in many cellular processes, including, but not limited to, DNA synthesis, respiration, and key metabolic reactions. Both iron overload and iron deficiency can lead to cytotoxicity and cell growth arrest/apoptosis, respectfully. Tight regulation of iron uptake, distribution, and export must be achieved to maintain iron homeostasis, which is mediated through transport, storage, and regulatory proteins. An iron-sensing switch is inherent in these regulatory proteins and cellular [Fe] controls synthesis of heme and iron-sulfur (Fe—S) clusters, in which these elements, in turn, regulate iron proteins. Disruption of iron homeostasis by mutations in these key proteins have been linked to human diseases, including, but not limited to, hereditary hyperferritinemia cataract syndrome, Friedreich's ataxia, X-linked siderablastic anemia, progressive neurodegenerative disease, and the like.

The iron responsive element (IRE)/iron regulatory protein (IRP) system is the most extensively studied mode of coordinated regulation in iron homeostasis. Trans-acting IRPs, through their metal availability sensors, interact with ferritin and transferrin receptor (TfR) IREs, which are conserved hairpin structures found in mRNA untranslated regions (UTRs), to control either mRNA stability or initiation of translation. TfR mediates cellular iron uptake by receptor mediated endocytosis of transferring-bound iron and ferritin stores cellular iron by its ability to bind up to 4,500 iron atoms. When iron is replete, Fe—S clusters assemble in IRP1, converting it to an aconistase, while IRP2, which does not contain an Fe—S cluster, is targeted for degradation. When iron is depleted, IRP1 loses its Fe—S cluster, enabling IRP1 and available IRP2 to bind to IREs in the 5′UTR of ferritin mRNA, inhibiting translation, and in the 3′UTR of TfR mRNA, stabilizing transcripts for efficient translation.

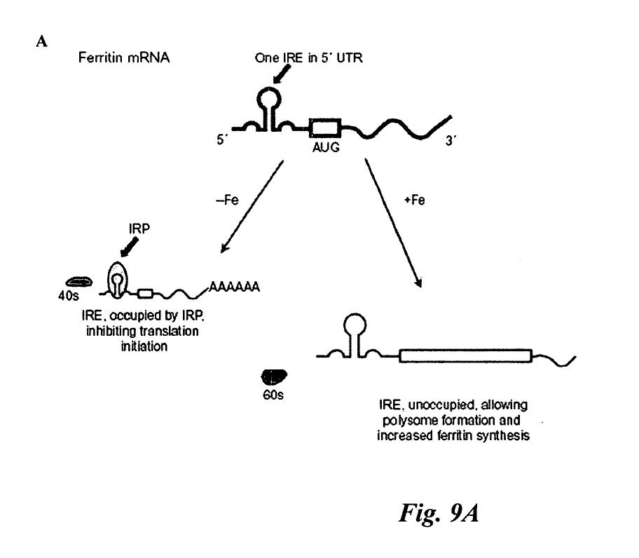

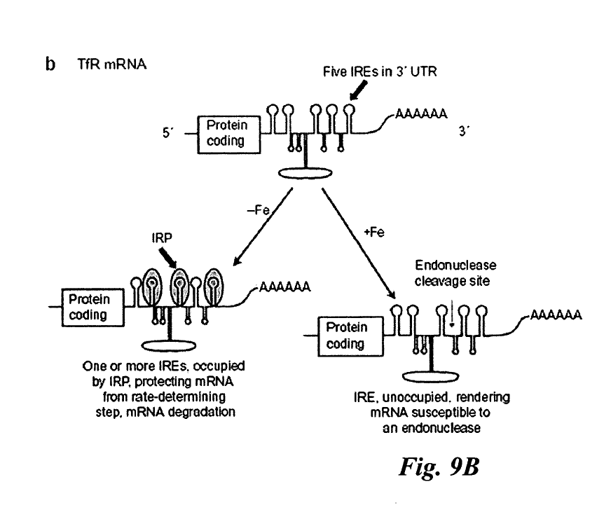

Referring now to FIGS. 9A and 9B, the regulation of ferritin translation and transferrin receptor (TfR) mRNA degradation by iron regulation protein (IRP) binding is illustrated. FIG. 9A shows that in iron-depleted cells, IRP binding to the iron responsive element (IRE) in the 5′ untranslated region (UTR) interferes with translation initiation. FIG. 9B shows that binding of IRPs to IREs in the TfR 3′UTR protects the transcript from endonucleolytic cleavage and degradation. This regulatory IRE/IRP system applies to at least fourteen other iron metabolism genes known to date.

Mitochondria are sites of Fe—S cluster and heme synthesis and contribute significantly to regulation of iron homeostasis. Hence, this organelle is where most of the cellular iron is routed. If Fe—S clusters within iron responsive proteins act as iron sensors, defects in Fe—S cluster synthesis or export from mitochondria should disrupt cellular iron homeostasis, possibly through imbalanced distribution of Fe—S clusters in both mitochondrial and cytosolic proteins, as well as mitochondrial iron overload/cytosolic iron depletion.

More particularly, as modeled by a human disease, e.g., X-linked sideroblastic anemia with ataxia (XLSA/A), studies with mouse models and human cells with deficient levels of mitochondrial inner membrane ATP-binding cassette (ABC) transporter ABCB7 have demonstrated mitochondrial iron overload, accumulation of protoporphyrin (precursor of heme), and dysregulation of IRP1 via impaired cytosolic Fe—S cluster assembly. Further, iron homostasis can alternatively be disrupted by suppression of Fe—S cluster scaffold protein, ISCU. As such, IRP1 activity as a post-transcriptional regulator appears to dominate when Fe—S cluster synthesis/stability is impaired by means of iron deprivation, i.e., treatment with iron chelators, and defective Fe—S cluster assembly and availability through transport.

ii. Metal Chelators for Enhancing Endogenous RNAi Activity

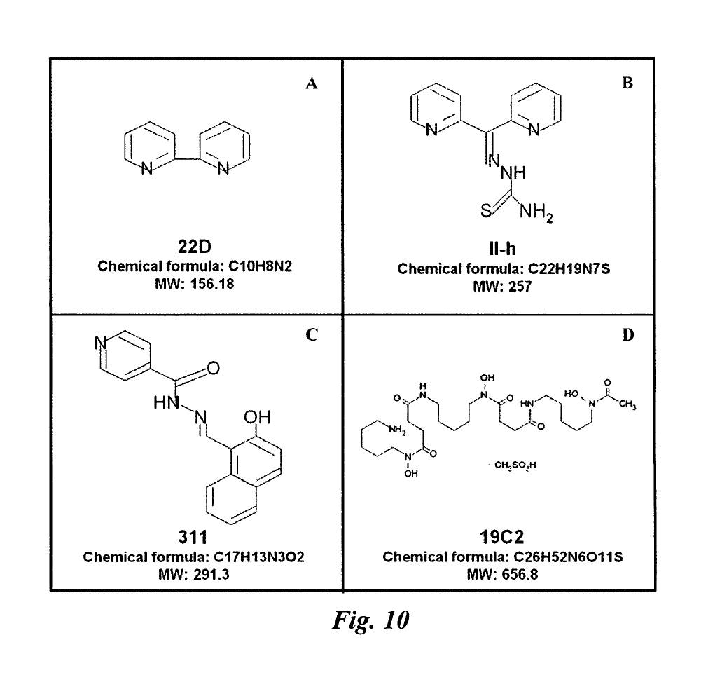

Methods and compositions are provided which provide metal chelating compounds, such as iron (Fe) chelating compounds, which enhance RNAi activity. More particularly, as described in more detail herein below in Example 9, four compounds having metal/Fe chelating ability were identified as enhancers of RNAi activity. As used herein, the terms “chelator” or “chelating agent” refer to a ligand capable of binding to or complexing with one or more metal ions to form a chelate complex. In a chelate complex, the metal ion is bound to two or more donor atoms of the chelating agent. Accordingly, a chelator or chelating agent can be a bidentate ligand or a multidentate ligand, e.g., a tridentate, quadridentate, pentadentate, or hexadentate ligand, or a macrocyclic ligand. One characteristic of a chelate complex is that, when bound to a chelator or chelating agent, the metal ion is chemically inert. The chemical structures of these compounds are provided in FIGS. 10A-10D and in Scheme 1.

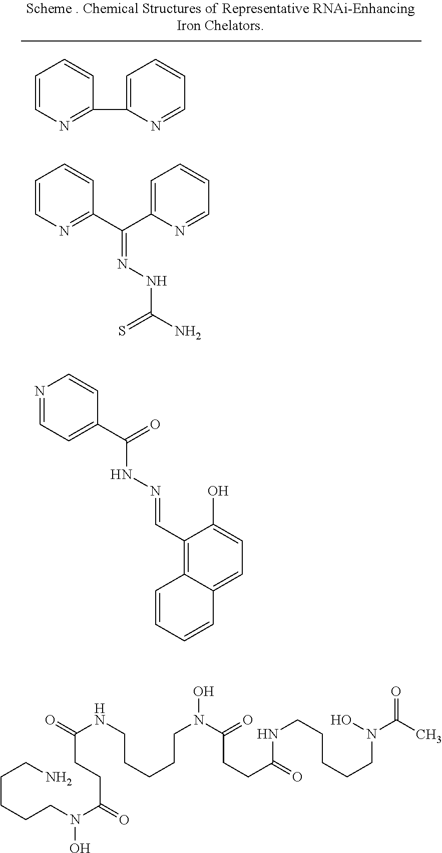

Each of the four compounds shown in Scheme 1 exhibited RNAi-enhancing activity that is more effective than Enoxacin (50 μM E50; 100 μM E100), even at low concentrations. Non-limiting examples of other iron chelators include, for example, those compounds described in further detail in section C(iii)(a) and C(iii)(b) herein below.

In some embodiments, the presently disclosed subject matter provides a method for decreasing the level of a target polynucleotide in a cell, the method comprising administering to the cell an effective amount of metal chelating agent, such as an iron chelating agent, and pharmaceutically and cosmetically acceptable salts thereof, wherein said cell further comprises at least one heterologous silencing element.

In some embodiments, the iron chelating agent is selected from a compound of Formula (n)-(q):

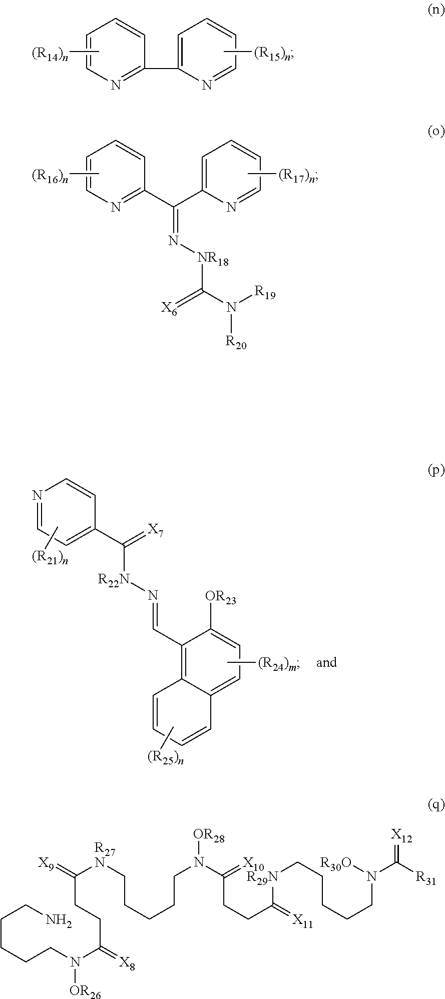

wherein:

m is an integer from 0 to 3;

n is an integer from 0 to 4;

R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;

R₁₈, R₂₂, R₂₇, and R₂₉ are each independently selected from the group consisting of H, alkyl, substituted alkyl, and hydroxyl;

R₂₃, R₂₆, R₂₈, and R₃₀ are each independently selected from the group consisting of H, alkyl, and substituted alkyl;

R₁₉, R₂₀, and R₃₁ are each independently selected from the group consisting of H, alkyl, substituted alkyl;

X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and

pharmaceutically and cosmetically acceptable salts thereof.

In some embodiments, the compound of Formula (n)-(q) is selected from the group consisting of:

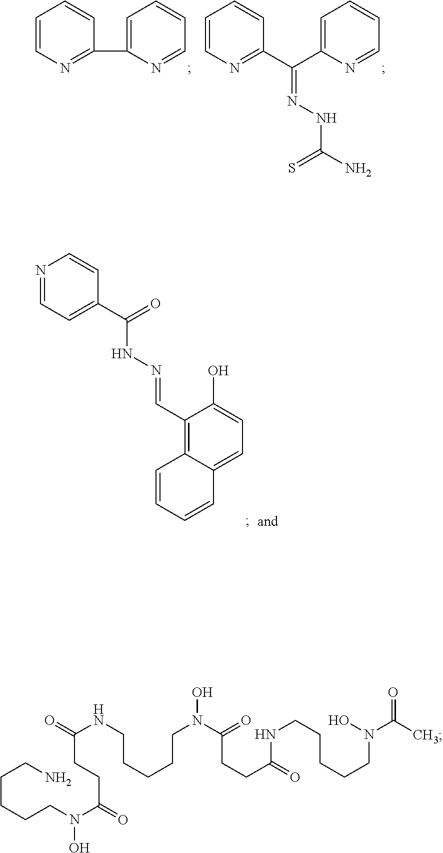

and

pharmaceutically and cosmetically acceptable salts thereof.

In some embodiments, the silencing element comprises an siRNA, an miRNA, a double stranded RNA, or a hairpin RNA. In some embodiments, the cell is in a subject.

In some embodiments, the presently disclosed subject matter provides a method for decreasing the level of a target polynucleotide in a cell, the method comprising administering to said cell a combination of an effective concentration of a polynucleotide comprising a heterologous silencing element and an effective amount of a metal chelating agent, such as an iron chelating agent, and pharmaceutically and cosmetically acceptable salts thereof.

In some embodiments, the iron chelating agent is selected from a compound of Formula (n)-(q):

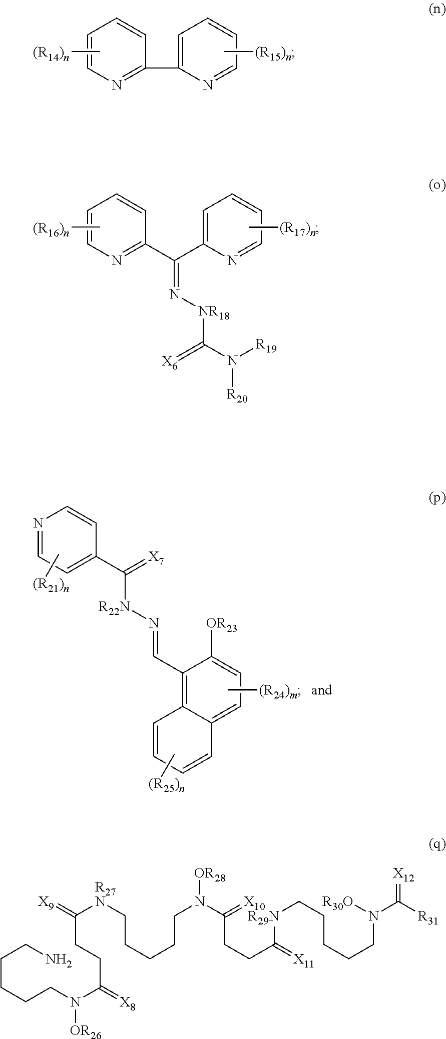

wherein:

m is an integer from 0 to 3;

n is an integer from 0 to 4;

R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;

R₁₈, R₂₂, R₂₇, and R₂₉ are each independently selected from the group consisting of H, alkyl, substituted alkyl, and hydroxyl;

R₂₃, R₂₆, R₂₈, and R₃₀ are each independently selected from the group consisting of H, alkyl, and substituted alkyl;

R₁₉, R₂₀, and R₃₁ are each independently selected from the group consisting of H, alkyl, substituted alkyl;

X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and

pharmaceutically and cosmetically acceptable salts thereof.

In some embodiments, the compound of Formula (n)-(q) is selected from the group consisting of

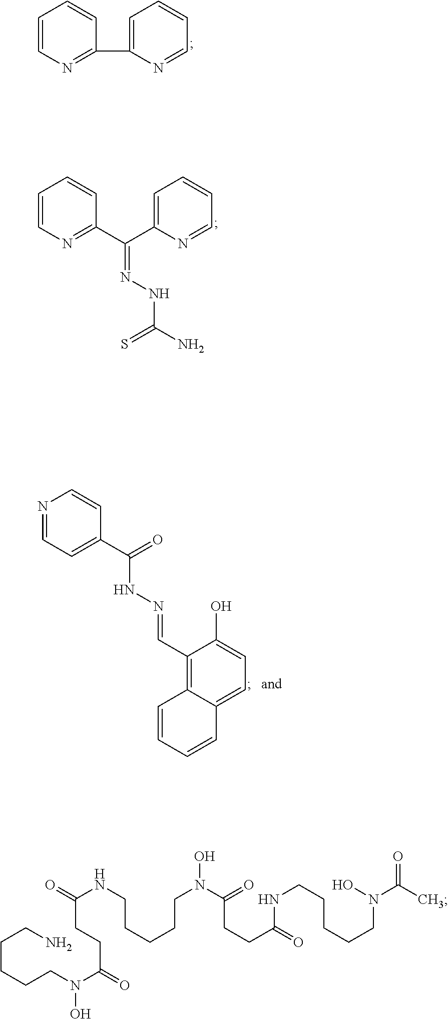

and

pharmaceutically and cosmetically acceptable salts thereof.

In some embodiments, the polynucleotide comprising the heterologous silencing element comprises an expression cassette encoding a siRNA, a miRNA, a dsRNA, or a hairpin RNA. In some embodiments, the polynucleotide is in a viral vector. In some embodiments, the polynucleotide comprises a siRNA, a miRNA, a dsRNA, or a hairpin RNA.

In some embodiments, the metal/Fe chelating agent and said heterologous silencing element are administered to the cell simultaneously or sequentially. In some embodiments, the cell is in a subject. In some embodiments, the cell is from a mammal.

In some embodiments, the presently disclosed subject matter provides a pharmaceutical or cosmetic composition comprising at least one of a compound of Formula (n)-(q) and a pharmaceutically or cosmetically acceptable carrier and one or more polynucleotides comprising a silencing element. In some embodiments, the silencing element comprises a siRNA, a miRNA, a double stranded RNA, or a hairpin RNA.

In some embodiments, the presently disclosed subject matter provides a method for decreasing the level of a target polynucleotide in a cell, the method comprising administering to the cell a combination of an effective concentration of a polynucleotide comprising a heterologous silencing element and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is a metal chelating agent, such as an iron chelating agent, and pharmaceutically and cosmetically acceptable salts thereof, wherein said cell further comprises at least one heterologous silencing element.

In some embodiments, the iron chelating agent is selected from a compound of Formula (n)-(q):

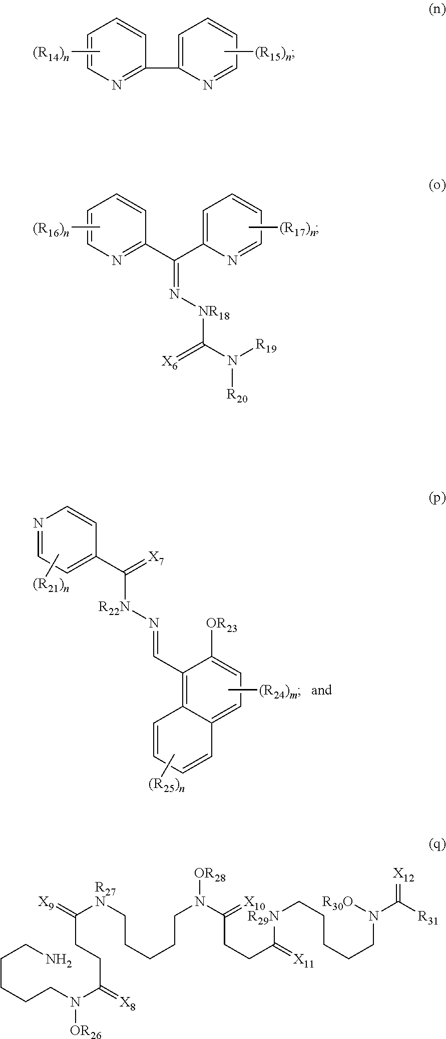

wherein:

m is an integer from 0 to 3;

n is an integer from 0 to 4;

R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;

R₁₈, R₂₂, R₂₇, and R₂₉ are each independently selected from the group consisting of H, alkyl, substituted alkyl, and hydroxyl;

R₂₃, R₂₆, R₂₈, and R₃₀ are each independently selected from the group consisting of H, alkyl, and substituted alkyl;

R₁₉, R₂₀, and R₃₁ are each independently selected from the group consisting of H, alkyl, substituted alkyl;

X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and

pharmaceutically and cosmetically acceptable salts thereof.

In some embodiments, the compound of Formula (n)-(q) is selected from the group consisting of:

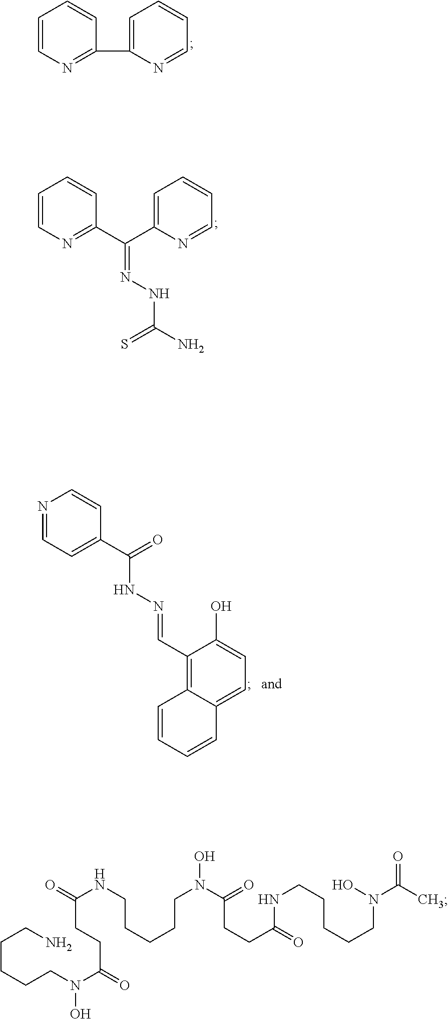

and

pharmaceutically and cosmetically acceptable salts thereof.

In some embodiments, the RNAi enhancer comprises enoxacin, or a derivative or analog thereof. In some embodiments, the silencing element comprises an siRNA, an miRNA, a double stranded RNA, or a hairpin RNA. In some embodiments, the cell is in a subject.

In some embodiments, the presently disclosed subject matter provides a method for decreasing the level of a target polynucleotide in a cell, the method comprising administering to said cell a combination of an effective concentration of a polynucleotide comprising a heterologous silencing element and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is a metal chelating agent, such as an iron chelating agent, and pharmaceutically and cosmetically acceptable salts thereof.

In some embodiments, the iron chelating agent is selected from a compound of Formula (n)-(q):

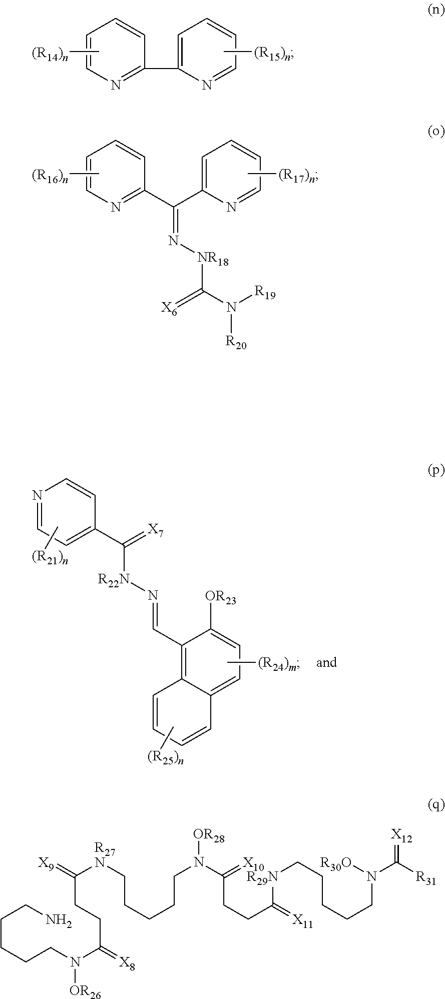

wherein:

m is an integer from 0 to 3;

n is an integer from 0 to 4;

R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;

R₁₈, R₂₂, R₂₇, and R₂₉ are each independently selected from the group consisting of H, alkyl, substituted alkyl, and hydroxyl;

R₂₃, R₂₆, R₂₈, and R₃₀ are each independently selected from the group consisting of H, alkyl, and substituted alkyl;

R₁₉, R₂₀, and R₃₁ are each independently selected from the group consisting of H, alkyl, substituted alkyl;

X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and

pharmaceutically and cosmetically acceptable salts thereof.

In some embodiments, the compound of Formula (n)-(q) is selected from the group consisting of:

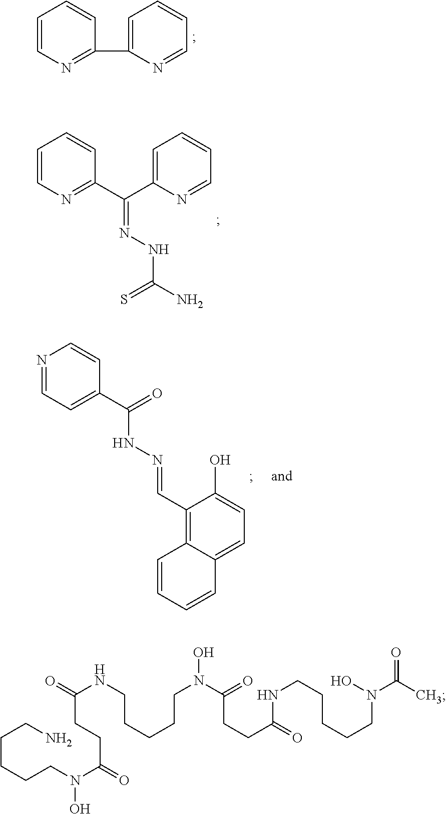

and

pharmaceutically and cosmetically acceptable salts thereof.

In some embodiments, at least one of the first RNAi enhancer and the at least one second RNAi enhancer comprises enoxacin, or a derivative or analog thereof. In some embodiments, the polynucleotide comprising the heterologous silencing element comprises an expression cassette encoding a siRNA, a miRNA, a dsRNA, or a hairpin RNA. In some embodiments, the polynucleotide is in a viral vector. In some embodiments, the polynucleotide comprises a siRNA, a miRNA, a dsRNA, or a hairpin RNA.

In some embodiments, the first RNAi enhancer and the at least one second RNAi enhancer and said heterologous silencing element are administered to the cell simultaneously or sequentially. In some embodiments, the cell is in a subject. In some embodiments, the cell is from a mammal

In some embodiments, the presently disclosed subject matter provides a pharmaceutical or cosmetic composition comprising at a first RNAi enhancer in combination with at least one second RNAi enhancer, wherein at least one of the first RNAi enhancer and the second RNAi enhancer is selected from the group consisting of a compound of Formula (a)-(q), and derivatives and analogs thereof, and a pharmaceutically or cosmetically acceptable carrier and one or more polynucleotides comprising a silencing element.

In some embodiments, the pharmaceutical or cosmetic composition of claim 99, wherein said silencing element comprises a siRNA, a miRNA, a double stranded RNA, or a hairpin RNA.

As described in more detail herein below, metal chelators are currently being tested clinically to treat cancer and neurodegenerative diseases. Accordingly, the presently disclosed subject matter suggests that metal chelators could be combined with siRNAs, to achieve a synergistic effect to treat these two types of diseases.

iii. Methods of Treating Cancer and Neurodegenerative Diseases with Metal Chelators in Combination with siRNAs

Iron chelators can bind Fe to render Fe chemically inert and unable to participate in disease progression. Chelating agents can be designed for either Fe^{II} or Fe^{III} oxidative states by selecting the donor atoms of the ligand. Generally, chelators that prefer Fe^{II} include so called “soft” donor atoms, including, but not limited to nitrogen and sulfur. Further, Fe^{II} chelators also have a relatively high affinity for other biologically-important metals, such as Cu^{II} and Zn^{II}. In contrast, chelators that prefer Fe^{III} generally include so called “hard” donor atoms, including, but not limited to oxygen. Fe^{III} chelators also bind Fe^{II} under most physiological conditions.

Generally, metal chelators suitable for treating neurodegenerative diseases include one or more of the following properties: (a) a strong affinity for Fe^{II} or Fe^{III}; (b) low molecular weight; (c) sufficiently high lipophilicity to facilitate permeation of cell membranes and the blood-brain barrier; (d) oral availability; and (e) low toxicity.

In vitro and/or in vivo screening of potential Fe chelator compounds can be used to determine Fe chelation efficiency. See Whitnall, M., et al., Semin. Pediatr. Neurol., 13:186-197 (2006), and references cited therein, all of which are incorporated by reference in their entirety.

a. Methods of Treating Cancers with Metal Chelators

A variety of structurally distinct metal, e.g., iron, chelators have been identified as potential anti-cancer agents. See, e.g., Le, N. T. V. and Richardson, D. R., Carcinogenesis, 24(6):1045-1058 (2003); Richardson, D. R., Critical Reviews in Oncology/Hematology, 42:267-281 (2002), and references cited therein, all of which are included herein by reference in their entirety. Such compounds find use in the various methods and compositions disclosed herein. For example, the following iron chelators or general chemical classes of iron chelators have been shown to have anti-proliferative activity: desferrioxamine (DFO); thiosemicarbazones, e.g., Triapine® (3-aminopyridine-2-carboxaldehyde thiosemicarbazone, Vion Pharmaceuticals, New Haven, Conn.) and 5-hydroxypyridine-2-carboxaldehyde thiosemicarbazone (5-HP), aroylhydrazones, e.g., pyridoxal isonicotinoyl hydrazone (P1H) and 2-hydroxy-1-naphthylaldehyde (311), tachpyridine (Tachpyr), and O-trensox (OTX). The chemical structures of these representative Fe chelators are provided in Scheme 2.

More particularly, in vitro and in vivo studies, as well as clinical trials, have demonstrated that some aggressive tumors, e.g., neuroblastoma (NB) and leukemia, are sensitive to Fe chelation therapy by desferrioxamine (DFO), a hexadentate Fe chelator that forms a 1:1 ligand-metal complex. See Richardson, D. R., Critical Reviews in Oncology/Hematology, 42:267-281 (2002).

Further, Fe chelators belonging to the chemical class of thiosemicarbazones, tridentate ligands, including Triapine®, have been extensively studied in relation to their use as anti-cancer agents. Representative thiosemicarbazones include, but are not limited to, 1-formylisoquinoline thiosemicarbazone, 2-formylpyridine thiosemicarbazone, 2-keto-3-ethoxy-butyraldehyde bis(thiosemicarbazone), α-(N)-heterocyclic carboxaldehyde thiosemicarbazones (HCTs), including 5-hydroxypyridine-2-carboxaldehyde thiosemicarbazone (5-HP) and 3-aminopyridine-2-carboxaldehyde thiosemicarbazone (3-APT or Triapine®). For example, Triapine® has activity against a variety of tumor cell models. Id.

Aroylhydrazones also are tridentate ligands, which have been shown to exhibit Fe chelation-dependent anti-tumor activity in cell culture studies. Id. Representative aroylhydrazone Fe chelators include, but are not limited to salicylaldehyde benzoyl hydrazone, 2-hydroxy-1-naphthylaldehyde, and analogues or derivatives thereof. For example, 2-hydroxy-1-naphthylaldehyde isonicotinoyl hydrazone (311) has shown very high Fe chelation efficiency and anti-proliferation activity. Id.

Tachpyridine, a hexadentate ligand that binds Fe, has been shown to inhibit ferritin synthesis and the proliferation of bladder cancer cells in culture. Id. Further, O-Trensox, which is based on three linked molecules of the potent Fe chelator 8-hydroxyquinoline is being investigated as an anti-tumor agent. Id.

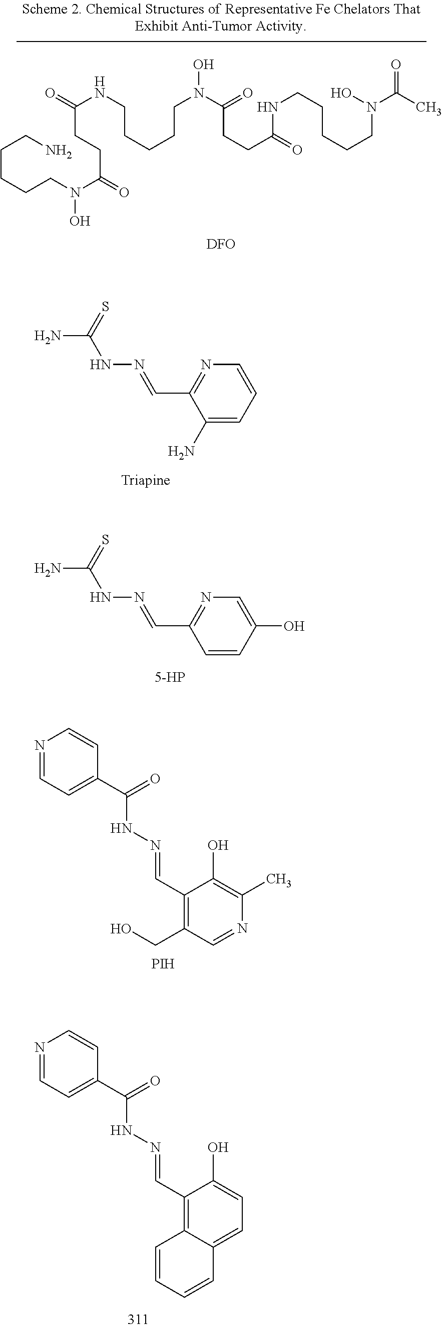

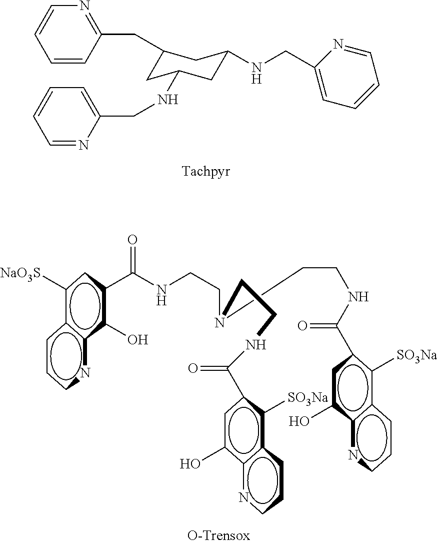

b. Methods of Treating Neurodegenerative Diseases with Metal Chelators

Iron accumulation and iron-related oxidative stress has been implicated in neurodegenerative diseases including, but not limited to, Huntington disease, Alzheimer's disease, Parkinson's disease, and Friedreich's atazia. See Whitnall, M., et al., Semin. Pediatr. Neurol., 13:186-197 (2006), and references cited therein, all of which are incorporated by reference in their entirety. Disruption of iron homeostasis also has been linked to hereditary hyperferritinemia cataract syndrome, X-linked siderablastic anemia, and progressive neurodegenerative disease. The involvement of Fe and related metals, e.g., copper and zinc, in these neurodegenerative diseases has provided a rationale for developing therapeutical strategies including metal-binding drugs, i.e., chelators. Id.

DFO, PIH, 8-hydroxyquinoline (8HQ), and 5-chloro-7-iodo-8-hydroxyquinoline (CQ) have shown potential for treating neurodegenerative diseases. Id. Chemical structures of these representative Fe chelators are provided in Scheme 3.

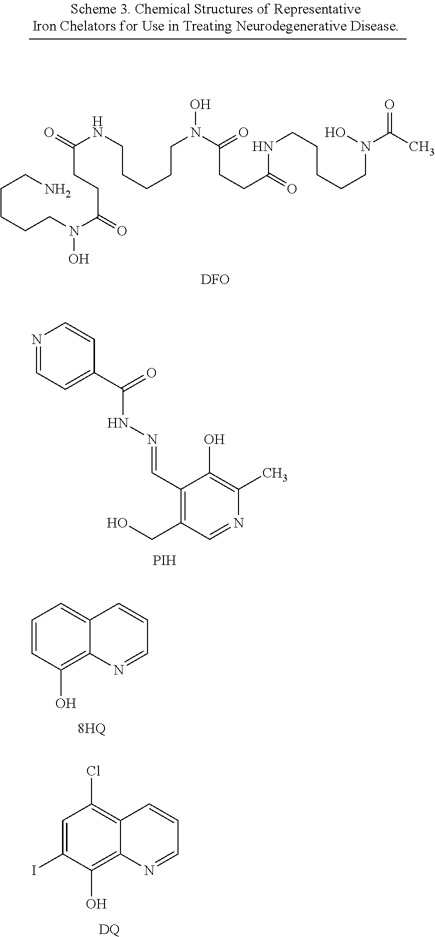

Further, the potential of analogs of 2-pyridylcarboxaldehyde isonicotinoyl hydrazone (PCIH), including, but not limited to, 2-pyridylcarboxaldehyde benzoyl hydrazone (PCBH); 2-pyridylcarboxaldehyde-m-bromobenzoyl hydrazone (PCBBBH); 2-pyridylcarboxaldehyde-p-aminobenzoyl hydrazone (PCAH); and 2-pyridylcarboxaldehyde-p-hydroxybenzoyl hydrazone (PCHH) for treating neurodegenerative disease has been investigated. Id. Chemical structures of representative analogs of PCIH are provided in Scheme 4.

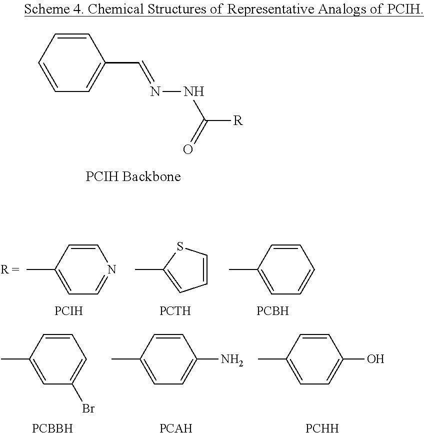

c. Presently Disclosed Methods

In some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of an metal chelating agent, and more particularly, an iron chelating agent, and pharmaceutically and cosmetically acceptable salts thereof.

In some embodiments, the iron chelating agent is selected from a compound of Formula (n)-(q):

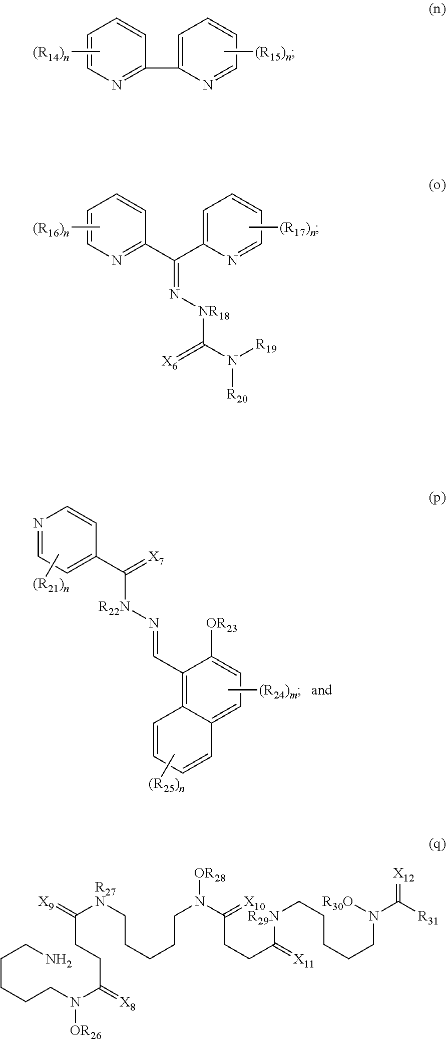

wherein:

m is an integer from 0 to 3;

n is an integer from 0 to 4;

R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;

R₁₈, R₂₂, R₂₇, and R₂₉ are each independently selected from the group consisting of H, alkyl, substituted alkyl, and hydroxyl;

R₂₃, R₂₆, R₂₈, and R₃₀ are each independently selected from the group consisting of H, alkyl, and substituted alkyl;

R₁₉, R₂₀, and R₃₁ are each independently selected from the group consisting of H, alkyl, substituted alkyl;

X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and

pharmaceutically and cosmetically acceptable salts thereof.

In some embodiments, the compound of Formula (a)-(q) is selected from the group consisting of:

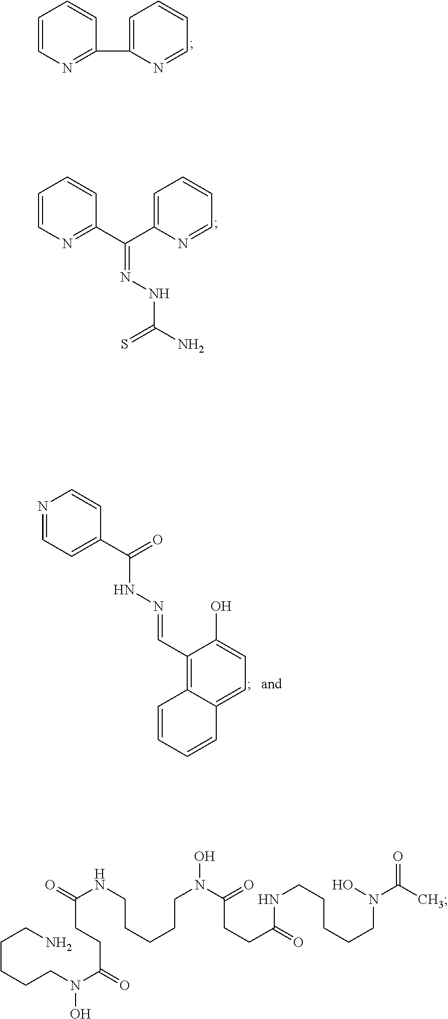

and

pharmaceutically and cosmetically acceptable salts thereof.

In some embodiments, the disease state is a cancer. In some embodiments, the cancer is selected from the group consisting of neuroblastoma (NB) and leukemia.

In some embodiments, the disease state comprises a neurodegenerative disorder. In some embodiments, the neurodegenerative disease is selected from the group consisting of Huntington disease, Alzheimer's disease, Parkinson's disease, Friedreich's atazia, hereditary hyperferritinemia cataract syndrome, X-linked siderablastic anemia, and progressive neurodegenerative disease.

In some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is an iron chelating agent, and pharmaceutically and cosmetically acceptable salts thereof.

In some embodiments, the iron chelating agent is selected from a compound of Formula (n)-(q):

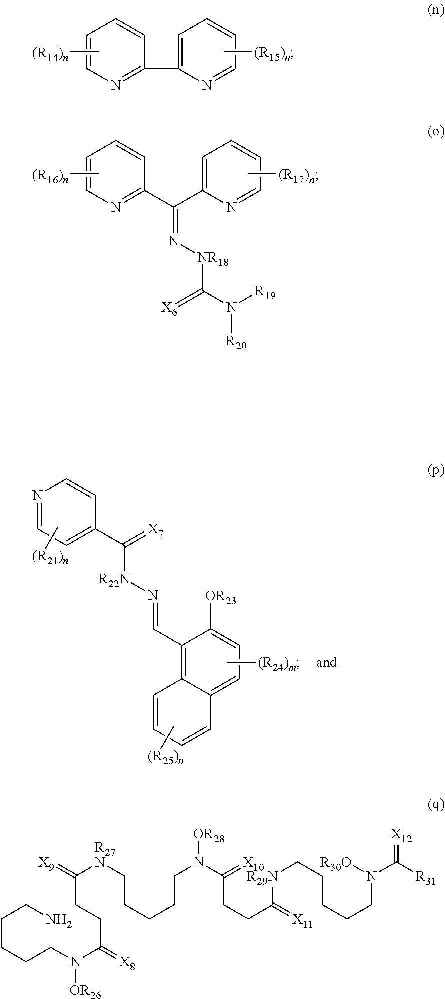

wherein:

m is an integer from 0 to 3;

n is an integer from 0 to 4;

R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;

R₁₈, R₂₂, R₂₇, and R₂₉ are each independently selected from the group consisting of H, alkyl, substituted alkyl, and hydroxyl;

R₂₃, R₂₆, R₂₈, and R₃₀ are each independently selected from the group consisting of H, alkyl, and substituted alkyl;

R₁₉, R₂₀, and R₃₁ are each independently selected from the group consisting of H, alkyl, substituted alkyl;

X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and

pharmaceutically and cosmetically acceptable salts thereof.

In some embodiments, the compound of Formula (n)-(q) is selected from the group consisting of:

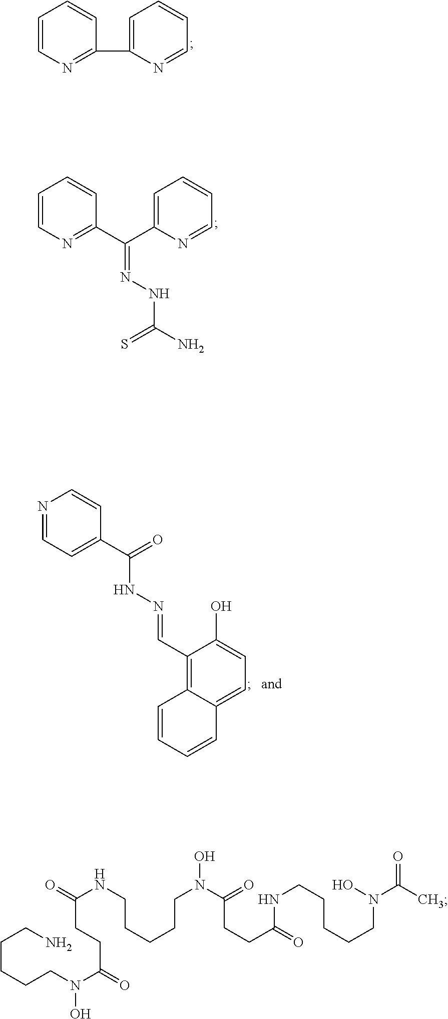

and

pharmaceutically and cosmetically acceptable salts thereof.

In some embodiments, at least one of the first RNAi enhancer and the at least one second RNAi enhancer comprises enoxacin, or derivatives or analogs thereof.

In some embodiments, the disease state is a cancer. In some embodiments, the cancer is selected from the group consisting of neuroblastoma (NB) and leukemia.

In some embodiments, the disease state comprises a neurodegenerative disorder. In some embodiments, the neurodegenerative disease is selected from the group consisting of Huntington disease, Alzheimer's disease, Parkinson's disease, Friedreich's atazia, hereditary hyperferritinemia cataract syndrome, X-linked siderablastic anemia, and progressive neurodegenerative disease.

D. Definition of Chemical Terms

While the following terms are believed to be well understood by one of ordinary skill in the art, the following definitions are set forth to facilitate explanation of the presently disclosed subject matter.

When the term “independently selected” is used, the substituents being referred to (e.g., R groups, such as groups R₁, R₂, and the like, or groups X₁ and X₂), can be identical or different. For example, both R₁ and R₂ can be substituted alkyls, or R₁ can be hydrogen and R₂ can be a substituted alkyl, and the like.

A named “R” or “X” group will generally have the structure that is recognized in the art as corresponding to a group having that name, unless specified otherwise herein. For the purposes of illustration, certain representative “R” and “X” groups as set forth above are defined below. These definitions are intended to supplement and illustrate, not preclude, the definitions that would be apparent to one of ordinary skill in the art upon review of the present disclosure.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, aryl, substituted aryl, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, such as a 3- to 7-member substituted or unsubstituted cycloalkyl ring system, including one or more heteroatoms, which can be the same or different, and are selected from the group consisting of N, O, and S, and optionally can include one or more double bonds. The cycloheteroalkyl ring can be optionally fused to or otherwise attached to other cycloheteroalkyl rings and/or non-aromatic hydrocarbon rings. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The term “alkenyl” as used herein refers to a straight or branched hydrocarbon of a designed number of carbon atoms containing at least one carbon-carbon double bond. Examples of “alkenyl” include vinyl, allyl, 2-methyl-3-heptene, and the like.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to an aromatic ring system, such as, but not limited to a 5- or 6-member ring system, including one or more heteroatoms, which can be the same or different, and are selected from the group consisting of N, O, and S. The heteroaryl ring can be fused or otherwise attached to one or more heteroaryl rings, aromatic or non-aromatic hydrocarbon rings, or heterocycloalkyl rings. Representative heteroaryl ring systems include, but are not limited to, pyridyl, pyrimidyl, pyrrolyl, pyrazolyl, azolyl, oxazolyl, isoxazolyl, oxadiazolyl, thiazolyl, isothiazolyl, imidazolyl, furanyl, thienyl, quinolinyl, isoquinolinyl, indolinyl, indolyl, benzothienyl, benzothiazolyl, enzofuranyl, benzimidazolyl, benzisoxazolyl, benzopyrazolyl, triazolyl, tetrazolyl, and the like.

A structure represented generally by the formula, wherein the ring structure can be aromatic or non-aromatic:

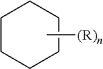

as used herein refers to a ring structure, for example, but not limited to a 3-carbon, a 4-carbon, a 5-carbon, a 6-carbon, and the like, aliphatic and/or aromatic cyclic compound, including a saturated ring structure, a partially saturated ring structure, and an unsaturated ring structure as defined herein, comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure above where n is 0 to 2 would comprise compound groups including, but not limited to:

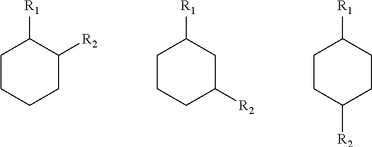

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

When a named atom of an aromatic ring or a heterocyclic aromatic ring is defined as being “absent,” the named atom is replaced by a direct bond.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Alkoxyl” refers to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

The term “alkyl-thio-alkyl” as used herein refers to an alkyl-5-alkyl thioether, for example, a methylthiomethyl or a methylthioethyl group.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl. “Aryloxycarbonyl” refers to an aryl-O—CO— group. Exemplary aryloxycarbonyl groups include phenoxy- and naphthoxy-carbonyl. “Aralkoxycarbonyl” refers to an aralkyl-O—CO— group. An exemplary aralkoxycarbonyl group is benzyloxycarbonyl.

“Carbamoyl” refers to an H₂N—CO— group. “Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The term “alkylamino” refers to an —NHR group wherein R is an alkyl group and/or a substituted alkyl group as previously described. Exemplary alkylamino groups include methylamino, ethylamino, and the like.

“Dialkylamino” refers to an —NRR′ group wherein each of R and R′ is independently an alkyl group and/or a substituted alkyl group as previously described. Exemplary dialkylamino groups include ethylmethylamino, dimethylamino, and diethylamino.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

As used herein, an “analog” refers to a chemical compound in which one or more individual atoms or functional groups of a parent compound have been replaced, either with a different atom or with a different functional group. For example, thiophene is an analog of furan, in which the oxygen atom of the five-membered ring is replaced by a sulfur atom.

As used herein, a “derivative” refers to a chemical compound which is derived from or obtained from a parent compound and contains essential elements of the parent compound but typically has one or more different functional groups. Such functional groups can be added to a parent compound, for example, to improve the molecule's solubility, absorption, biological half life, and the like, or to decrease the toxicity of the molecule, eliminate or attenuate any undesirable side effect of the molecule, and the like. An example of a derivative is an ester or amide of a parent compound having a carboxylic acid functional group.

E. Pharmaceutically or Cosmetically Acceptable Salts

Additionally, the active compounds as described herein can be administered as a pharmaceutically or cosmetically acceptable salt. The phrases “pharmaceutically acceptable salt(s)” or “cosmetically acceptable salt(s),” as used herein, means those salts of the presently disclosed compounds that are safe and effective for use in a subject and that possess the desired biological activity. Pharmaceutically or cosmetically acceptable salts include salts of acidic or basic groups present in compounds of the invention. Pharmaceutically or cosmetically acceptable acid addition salts include, but are not limited to, hydrochloride, hydrobromide, hydroiodide, nitrate, sulfate, bisulfate, phosphate, acid phosphate, borate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, pamoate (i.e., 1,1′-methylene-bis-(2-hydroxy-3-naphthoate)), mesylate salts. Certain of the presently disclosed compounds can form pharmaceutically or cosmetically acceptable salts with various amino acids. Suitable base salts include, but are not limited to, aluminum, calcium, lithium, magnesium, potassium, sodium, zinc, and diethanolamine salts. For a review on pharmaceutically acceptable salts see Berge et al., 66 J. Pharm. Sci. 1-19 (1977), which is incorporated herein by reference. The salts of the compounds described herein can be prepared, for example, by reacting the appropriate equivalent of the compound with the desired acid or base in solution. After the reaction is complete, the salts are crystallized from solution by the addition of an appropriate amount of solvent in which the salt is insoluble.

F. Pharmaceutical or Cosmetic Formulations and Compositions

RNAi can be used in mammalian cells grown in culture and in mammalian organisms, e.g., for functional studies of genes. In addition, animal studies have indicated that RNAi-inducing agents are likely to have therapeutic applications. Thus, compounds that inhibit or activate RNAi are useful in mammalian tissue culture systems, in animal studies, and for therapeutic purposes. The presently disclosed subject matter therefore provides pharmaceutical or cosmetic compositions comprising one or more silencing elements targeted to one or more specific genes and at least one enhancing compound, including, but not limited to, at least one presently disclosed compound, as described hereinabove.

The presently disclosed compositions (e.g., compounds that activate or inhibit an RNAi pathway) can be formulated for delivery, i.e., administering to the subject, by any available route including, but not limited, to parenteral (e.g., intravenous), intradermal, subcutaneous, oral, nasal, bronchial, opthalmic, transdermal (topical), transmucosal, rectal, and vaginal routes. Preferred routes of delivery include parenteral, transmucosal, nasal, bronchial, vaginal, and oral. The presently disclosed pharmaceutical or cosmetic compositions also can include an RNAi-inducing agent in combination with a pharmaceutically or cosmetically acceptable carrier. As used herein the terms “pharmaceutically acceptable carrier” or “cosmetically acceptable carriers” include solvents, dispersion media, coatings, antibacterial and antifungal agents, isotonic and absorption delaying agents, and the like, compatible with pharmaceutical or cosmetic administration. Supplementary active compounds also can be incorporated into the compositions.

As one of ordinary skill in the art would appreciate, a presently disclosed pharmaceutical or cosmetic composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical or cosmetic compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition should be sterile and should be fluid to the extent that easy syringability exists. Preferred pharmaceutical or cosmetic compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols, such as manitol or sorbitol, or sodium chloride in the composition. Prolonged absorption of the injectable composition can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral formulations generally include an inert diluent or an edible carrier. For the purpose of oral therapeutic administration, the active compound can be incorporated with excipients and used in the form of tablets, troches, or capsules, e.g., gelatin capsules. Oral formulations also can be prepared using a fluid carrier for use as a mouthwash. Pharmaceutically or cosmetically compatible binding agents, and/or adjuvant materials can be included as part of the composition. The tablets, pills, capsules, troches, and the like can contain any of the following ingredients, or compounds of a similar nature: a binder, such as microcrystalline cellulose, gum tragacanth or gelatin; an excipient, such as starch or lactose, a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring. Formulations for oral delivery can advantageously incorporate agents to improve stability within the gastrointestinal tract and/or to enhance absorption.

For administration by inhalation, the presently disclosed formulations and compositions are preferably delivered in the form of an aerosol spray from a pressured container or dispenser which contains a suitable propellant, e.g., a gas such as carbon dioxide, or a nebulizer. Liquid aerosols, dry powders, and the like, also can be used.

Systemic administration of the presently disclosed compositions also can be by transmucosal or transdermal means. For transmucosal or transdermal administration, penetrants appropriate to the barrier to be permeated are used in the composition. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds also can be prepared in the form of suppositories (e.g., with conventional suppository bases such as cocoa butter and other glycerides) or retention enemas for rectal delivery.

The presently disclosed compositions also can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions (including liposomes targeted to infected cells with monoclonal antibodies to viral antigens) also can be used as pharmaceutically or cosmetically acceptable carriers. Such suspensions can be prepared according to methods known to those skilled in the art, for example, as described in U.S. Pat. No. 4,522,811, which is incorporated herein by reference in its entirety.

It is advantageous to formulate oral or parenteral formulations in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical or cosmetic carrier.

Toxicity and therapeutic efficacy of such compounds can be determined by standard pharmaceutical or cosmetic procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the presently disclosed methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical or cosmetic composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical or cosmetic composition can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, and the like. The skilled artisan will appreciate that certain factors can influence the dosage and timing required to effectively treat a subject, including but not limited to the severity of the disease, disorder, or unwanted condition, previous treatments, the general health and/or age of the subject, and other diseases or unwanted conditions present. Generally, treatment of a subject can include a single treatment or, in many cases, can include a series of treatments. Further, treatment of a subject can include a single cosmetic application or, in some embodiments, can include a series of cosmetic applications.

Exemplary doses include milligram or microgram amounts of the inventive compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) For local administration (e.g., intranasal), smaller doses can be used. It is furthermore understood that appropriate doses of a compound depend upon its potency and can optionally be tailored to the particular recipient, for example, through administration of increasing doses until a preselected desired response is achieved. It is understood that the specific dose level for any particular animal subject can depend on a variety of factors including the activity of the specific compound employed, the age, body weight, general health, gender, and diet of the subject, the time of administration, the route of administration, the rate of excretion, any drug combination, and the degree of expression or activity to be modulated.

The presently disclosed compositions can be used for the treatment of nonhuman animals including, but not limited to, horses, swine, and birds. Accordingly, doses and methods of administration can be selected in accordance with known principles of veterinary pharmacology and medicine. Guidance for appropriate doses and methods of administration can be found, for example, in Adams, R. (ed.), Veterinary Pharmacology and Therapeutics, 8th edition, Iowa State University Press; ISBN: 0813817439; 2001.

The presently disclosed pharmaceutical or cosmetic compositions can be included in a container, pack, or dispenser together with instructions for administration.

Further, one of ordinary skill in the art upon review of the presently disclosed subject matter would appreciate that the presently disclosed compounds, including pharmaceutically or cosmetically acceptable salts and pharmaceutical or cosmetic compositions thereof, can be administered directly to a cell, a cell culture, a cell culture medium, a tissue, a tissue culture, a tissue culture medium, and the like. When referring to an RNA:adjuvant or suppressor, such as a compound of Formula (a)-(k) or Formula (n)-(q), or the RNA:suppressors of Formula (l)-(m), the term “administering,” and derivations thereof, comprises any method that allows for the compound to contact a cell. The presently disclosed compounds, or pharmaceutically or cosmetically acceptable salts or pharmaceutical or cosmetic compositions thereof, can be administered to (or contacted with) a cell or a tissue in vitro or ex vivo. The presently disclosed compounds, or pharmaceutically or cosmetically acceptable salts or pharmaceutical or cosmetic compositions thereof, also can be administered to (or contacted with) a cell or a tissue in vivo by administration to an individual subject, e.g., a patient, for example, by systemic administration (e.g., intravenous, intraperitoneal, intramuscular, subdermal, or intracranial administration) or topical application, as described elsewhere herein.

G. Methods for Treating a Disease State or Unwanted Condition

In some embodiments, the presently disclosed subject matter provides a method for treating a disease state or unwanted condition. RNAi can be used as a therapeutic approach for treating diseases that result from aberrant protein synthesis, for example diseases arising from genetic disorders or viral infections. In such embodiments, inhibiting or eliminating a target mRNA would result in a significant decrease in the expression level of a specific protein. Because RNAi can be used to target virtually any protein, RNAi-based therapies can be developed for almost any therapeutic area. Such approaches therefore have broad applicability, high therapeutic specificity, and target RNA destruction resulting in a decrease or termination of disease progression.

Accordingly, in some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a compound of Formula (a)-(k) and/or the various metal chelating agents, such as the iron chelating agents, including compounds of Formula (n)-(q), as disclosed herein, and pharmaceutically or cosmetically acceptable salts thereof. In some embodiments, the disease state comprises a viral infection. In some embodiments, the disease state comprises a genetic disorder.

In some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is selected from the group consisting of a compound of Formula (a)-(k) and/or the various metal chelating agents, such as the iron chelating agents, including compounds of Formula (n)-(q), and derivatives and analogs thereof.

In some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a compound of Formula (l)-(m), as disclosed herein, and pharmaceutically or cosmetically acceptable salts thereof. In some embodiments, the disease state comprises a viral infection. In some embodiments, the disease state comprises a genetic disorder.

In some embodiments, the presently disclosed subject matter provides a method for treating a disease state or unwanted condition, the method comprising administering to a subject in need of treatment thereof an effective amount of a polynucleotide comprising a silencing element, which, when administered to said subject, decreases the level of the target polynucleotide and an effective amount of at least one compound of Formula (l)-(m) and at least one RNAi enhancer. In some embodiments, the RNAi enhancer comprises enoxacin, or a derivative or analog thereof. In some embodiments, the disease state comprises a viral infection. In some embodiments, the disease state comprises a genetic disorder.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

To identify the chemical compounds that modulate the siRNA and miRNA pathway, a screening strategy was used. A human 293-cell line stably expressing a reporter gene, GFP, was used. This cell line was further transfected with a construct that expresses siRNA hairpin against GFP, which could knock down the expression of GFP. Individual clones were isolated with greatly reduced GFP expression. To verify that the decrease of GFP expression is due to GFP siRNA, 2-0-methyl RNA was transfected against GFP siRNA into those cells, which has been shown to block the siRNA effect previously, and found that GFP expression increased (FIG. 3, second panel). Using these clones, a screen was performed using The Spectrum Collection, which contains 2000 biologically active and structurally diverse compounds from libraries of known drugs, experimental bioactives, and pure natural products. The compound trimethobenzamide was found to inhibit siRNA-mediated mRNA degradation and gene knock-down (FIG. 3, third panel). The compound enoxacin was found to enhance siRNA-mediated mRNA degradation and gene knock-down (FIG. 4).

### Example 2

Human 293 cells stably expressing a reporter gene, GFP, were further transfected with a construct that expresses an siRNA hairpin against GFP, which results in the knock-down of the expression of GFP. Transfected cells containing the siRNA suppressed GPF expression were split and then treated with an effective amount of enoxacin (FIG. 4, second panel) and were grown for a period of 24-48 hours. Untreated control cells are shown in FIG. 4, first panel.

### Example 3

FIG. 5 shows enhancement of mRNA-mediated mRNA degradation and gene knock-down by presently disclosed quinolone compounds, e.g., enoxacin, ciproflaxin, and ofloxacin. In this example, a luciferase reporter construct GL-3 was transfected into the cells expressing shRNA against Luciferase mRNA. Addition of the disclosed compounds increased the knock-down efficiency of shRNA against luciferase mRNA. More particularly, FIG. 5 shows the relative luciferase activity of GL-3 luciferase only; GL-3 luciferase and one of enoxacin, ciprofloxacin, and ofloxacin; GL-3 luciferase and short hairpin RNA specific for luciferase gene (shLuc) only; and GL-3 luciferase, shLuc, and one of enoxacin, ciprofloxacin, and ofloxacin.

### Example 4

FIG. 6 shows a schematic representation of a microRNA (miRNA) sensor in mammalian cells. More particularly, FIG. 6 shows reporter construct and selective translational suppression of reporter containing miR-30a-3p target sites. In FIG. 6, top left panel, the blue arrowheads in Luc-T30 indicate artificial target sequence against miR-30a-3p. Luc-AT30 has an artificial sequence complementary to that in Luc-T30. FIG. 6, bottom left panel, shows a dual luciferase assay of HepG2 cells. Relative luciferase activity was calculated by dividing the reporter (Firefly) luciferase activity with co-transfected Renilla luciferase activity. Luciferase activity was decreased by transfecting Luc-T30 in HepG2 (orange bars) without changes in the amount of its mRNA (blue bars). Error bars represent the standard deviation (SD) of three triplicate experiments. FIG. 6a shows the design of an siRNA duplex against miR-30a precursor (siRNA-p) and a control siRNA duplex (siRNA-c). FIG. 6b shows that transfecting siRNA-p reversed the translational suppression of Luc-T30 (blue bar) while siRNA-c had no effect (orange bar). FIG. 6c shows that the recovery is siRNA-p concentration-dependent. Error bars represent the standard deviation (SD) from three triplicate experiments. **P=0.004; *****P=0.00001.

### Example 5

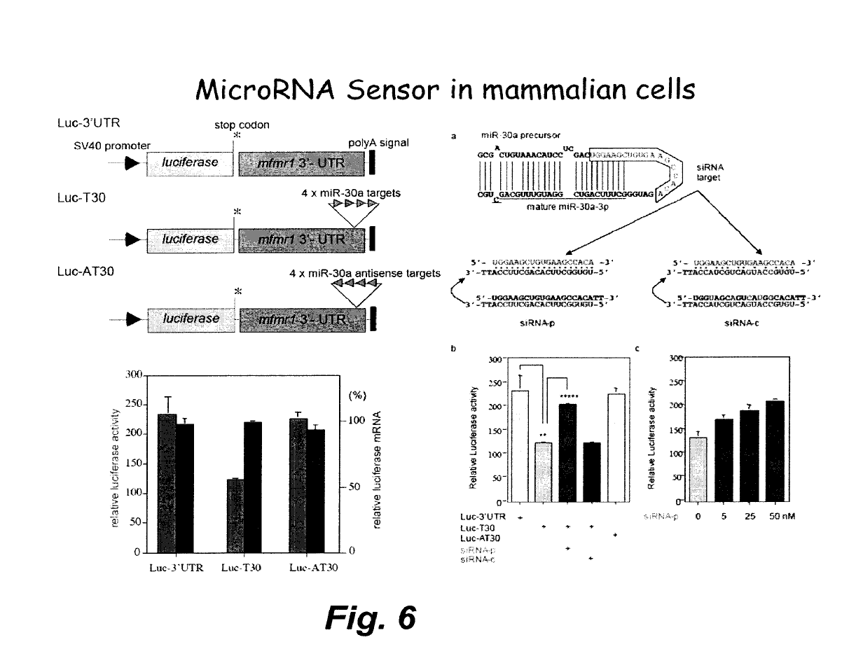

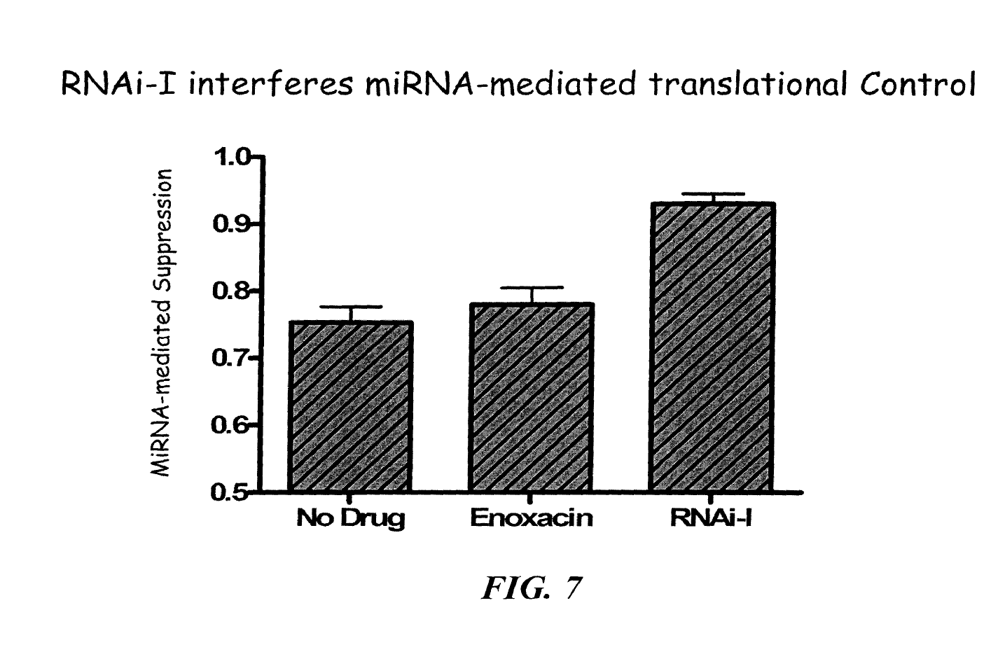

FIG. 7 shows the relative miRNA-mediated suppression exhibited by a presently disclosed RNAi inhibitor (RNAi-I) as compared to “no drug” and enoxacin. The reporter system described in FIG. 6 was used and the addition of RNAi-I released the miRNA-mediated translational suppression. This reporter contains the target sequence of miR-30. In the miR-30 system, the endogenously expressed miR-30 was used and, in this cell line, miR-30 has been processed completely. Thus, the addition of enoxacin had no effect. These results suggest that Enoxacin works upstream of the RISC complex.

### Example 6

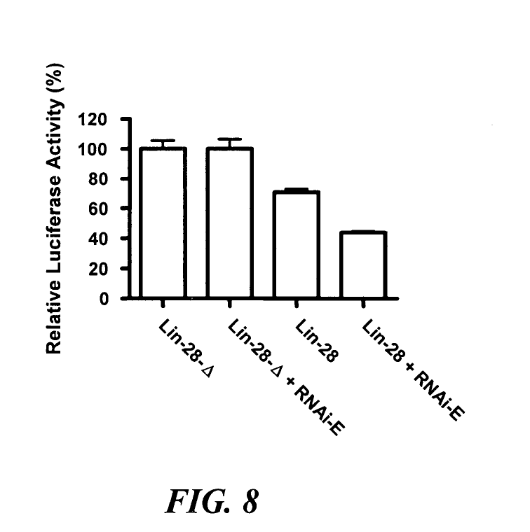

Referring now to FIG. 8, the 3′-UTR of Lin28 mRNA, a target mRNA of miR-125a, was inserted into the 3′ end of luciferase reporter gene. Lin28-Del is the construct without the target sequence of miR-125a. Lin28 or Lin28-Del was transfected into cells expressing miR-125a. Although RNAi-E had no effect on Lin28-Del, the translation of Lin28 was suppressed by the expression of miR-125a. The addition of RNAi-E further enhanced this suppression.

### Example 7

The compounds listed in Table 1 have been found by the presently disclosed methods to inhibit or enhance siRNA-mediated mRNA degradation.

TABLE 1
Compounds Found to Inhibit or Enhance siRNA-mediated mRNA Degradation.

| Compound | Chemical | | | Modulation |
|---|---|---|---|---|
| Name | Formula | Chemical Structure† | Position | Effect |
| Colforsin | C₂₂H₃₄O₇ | 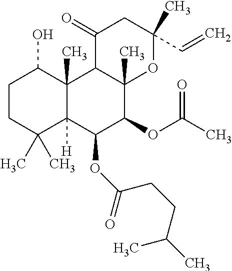 | 17-B2 | Inhibitor |
| Metameconine | C₁₀H₁₀O₄ | 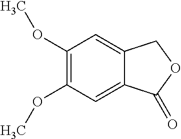 | 21-D3 | Inhibitor |
| Triprolidine Hydrochloride | C₁₉H₂₃ClN₂ | 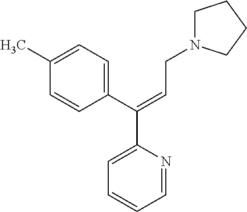 | 4-H5 | Enhancer |
| Dihydro- ptaeroxylin | C₁₅H₁₆O₄ | 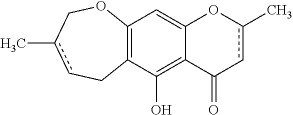 | 5-H10 | Enhancer |
| Fusidic Acid | C₃₁H₄₈O₆ | 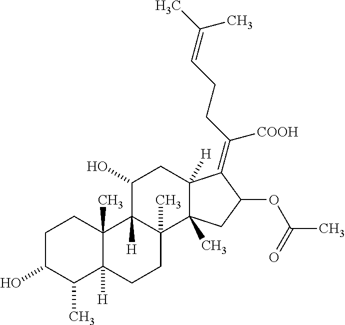 | 8-C9 | Enhancer |
| Fenbufen | C₁₆H₁₄O₃ | 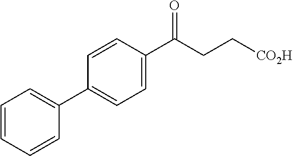 | 8-G4 | Enhancer |
| 3-beta- hydroxy- deoxodihydro- deoxygedunin | C₂₈H₃₈O₆ | 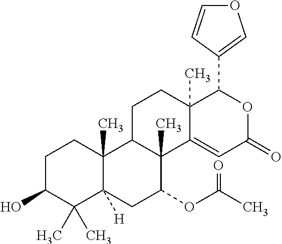 | 12-D8 | Enhancer |
| Deferoxamine Mesylate | C₂₆H₅₂N₆O₁₁S | 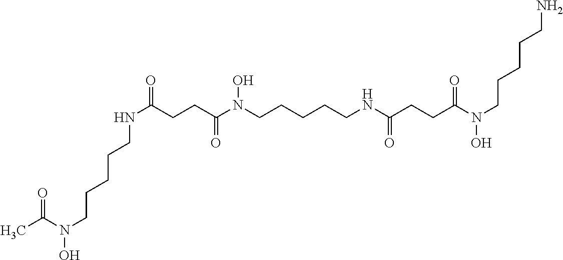 | 19-C2 | Enhancer |
| Thioguanine | C₅H₅N₅S | 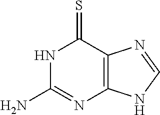 | 21-C11 | Enhancer |
| 2- aminomethyl- 1,4- benzodioxane hydrochloride (WB4101) | C₁₉H₂₄ClNO₅ | 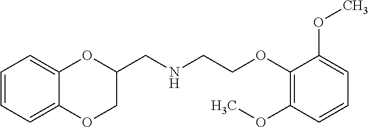 | 24-H11 | Enhancer |
| 3-alpha- hydroxy-3- deoxy- angloensic acid methyl ester | C₂₇H₃₆O₇ | 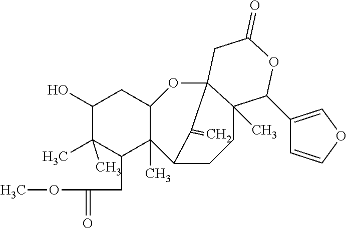 | 2-H8 | Enhancer |
| Lunarine | C₂₅H₃₁N₃O₄ | 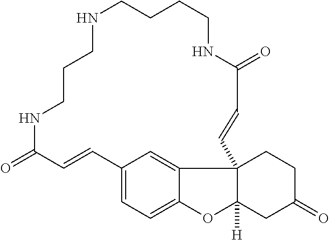 | 3-E7 | Enhancer |
| Bromocriptine Mesylate | C₃₃H₄₄BrN₅O₈S | 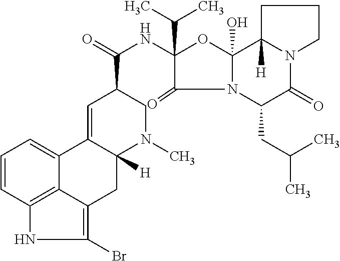 | 13-G8 | Enhancer |

†Specific salts not shown.

### Example 8

The presently disclosed chemical library also was screened using a plate reader to quantify the GFP signal intensity. The following screens were conducted: (1) repeating chemical screen using plate reader to quantify the GFP signal (50 mM each drug); (2) treating cells with 50 mM RNAi-I (30 mM each additional drug); and treating cells with 50 mM RNAi-E (30 mM each additional drug).

For each screen, two types of cells were used: Clone#3 and EGFP. Clone#3 cells are 293-EGFP-siGFP and could be used to identify the modulators of the RNAi pathway. EGFP cells are 293-EGFP only and were used as a control to eliminate the compounds that could kill the cells or interfere GFP signals that are not related to RNAi pathway. The results for each screen are summarized in Table 2 and Table 3, respectively.

In these three screens, compounds were identified that could either enhance or inhibit RNAi activity in Clone#3, but had no effect on EGFP cells, which would indicate that these compounds might specifically work on RNAi pathway. The modulation of RNAi activity by these compounds was calculated as the percentage of GFP signal to Clone#3 control cells.

TABLE 2
Screen of Chemical Library with Clone#3, 293-EGFP-siGFP Cells.

| | | | | | Clone#3 |
|---|---|---|---|---|---|
| | | | | | 293-EGFP- |
| | | | | Clone#3, | siGFP cells |
| | | | | 293-EGFP- | % GFP |
| | | | | siGFP cells | signal |
| | | | | GFP signal | intensity with |
| | | | | intensity | drug alone/ |
| Drug | | | | with Drug | Control |
| Position | Effect | Formula | Name | alone | (no drug) |
| 17-B2 | Inhibitor | C₂₂H₃₄O₇ | Colforsin | | |
| 21-D3 | Inhibitor | C₁₀H₁₀O₄ | Metameconine | | |
| 4-H5 | Enhancer | C₁₉H₂₃ClN₂ | Triprolidine Hydrochloride | 26791778.5 | 0.92917369 |
| 5-H10 | Enhancer | C₁₅H₁₆O₄ | Dihydroptaeroxylin | 26383349 | 0.91500883 |
| 8-C9 | Enhancer | C₃₁H₄₈O₆ | Fusidic Acid | 27803206.5 | 0.96425133 |
| 8-G4 | Enhancer | C₁₆H₁₄O₃ | Fenbufen | 26499027 | 0.91902069 |
| 12-D8 | Enhancer | C₂₈H₃₈O₆ | 3-beta-Hyroxydeoxo- | 17430963 | 0.752893 |
| | | | dihydrodeoxygedunin | | |
| 19-C2 | Enhancer | C₂₆H₅₂N₆O₁₁S | Deferoxamine Mesylate | 15874023.8 | 0.58892604 |
| 21-C11 | Enhancer | C₅H₅N₅S | Thioguanine | 11903224.8 | 0.44160946 |
| 24-H11 | Enhancer | C₁₉H₂₄CINO₅ | 2-Aminomethyl-1,4- | 23261710.5 | 0.85700161 |
| | | | Benzodioxane | | |
| | | | Hydrochloride (WB4101) | | |
| 2-H8 | Enhancer | C₂₇H₃₆O₇ | 3-alpha-Hydroxy-3- | 10997867 | 13375282 |
| | | | Deoxyangolensic acid | | |
| | | | methyl easter | | |
| 3-E7 | Enhancer | C₂₅H₃₁N₃O₄ | Lunarine | 9353700 | 15643152 |
| 13-G8 | Enhancer | C₃₃H₄₄BrN₅O₈S | Bromocriptine Mesylate | 7607590 | 14711690 |

| | | Clone#3, | | |
|---|---|---|---|---|
| | | 293-EGFP- | | |
| | | siGFP cells | | Clone#3, |
| | Clone#3, | % GFP | Clone#3, | 293-EGFP- |
| | 293-EGFP- | signal | 293-EGFP- | siGFP cells |
| | siGFP cells | intensity | siGFP cells | GFP signal |
| | GFP signal | with | GFP signal | intensity |
| | intensity | drug + RNAi- | intensity | without any |
| Drug | with Drug | E/Control | with | compounds |
| Position | and RNAi-E | (no drug) | RNAi-E | (control) |
| 17-B2 | | | | |
| 21-D3 | | | | |
| 4-H5 | 19639902.5 | 0.68113734 | 20289248 | 28833983 |
| 5-H10 | 17140237.8 | 0.59444572 | 20289248 | 28833983 |
| 8-C9 | 18581363.5 | 0.64442583 | 20289248 | 28833983 |
| 8-G4 | 17801864.5 | 0.61739179 | 20289248 | 28833983 |
| 12-D8 | 13998738 | 0.604646 | 16615771 | 23151967 |
| 19-C2 | 12987131 | 0.48182236 | 18695953 | 26954188.5 |
| 21-C11 | 9228044.75 | 0.34236032 | 18695953 | 26954189 |
| 24-H11 | 16441211.8 | 0.60572265 | 20015152 | 27143135 |
| 2-H8 | 82.23% | 10902446 | 81.51% | 129330172 |
| 3-E7 | 59.79% | 11171512 | 71.41% | 131010562 |
| 13-G8 | 51.71% | 10893798 | 74.05% | 132392840 |

TABLE 3
Screen of Chemical Library with EGFP, 293-EGFP Cells.

| | | | | | | EGFP, 293- | | EGFP, 293- | |
|---|---|---|---|---|---|---|---|---|---|
| | | | | EGFP, 293- | EGFP, 293- | EGFP cells | EGFP, 293- | EGFP cells | EGFP, 293- |
| Drug | | | | EGFP cells | EGFP cells | With | EGFP cells | RNAi-E | EGFP cells |
| Position | Effect | Formula | Name | Drug alone | Control % | RNAi-E | Control % | alone | Control |
| 17-B2 | Inhibitor | C₂₂H₃₄O₇ | Colforsin | | | | | | |
| 21-D3 | Inhibitor | C₁₀H₁₀O₄ | Metameconine | | | | | | |
| 4-H5 | Enhancer | C₁₉H₂₃CIN₂ | Triprolidine | 202102776 | 0.99069988 | 202110668 | 0.99073857 | 177574932 | 2.04E+08 |
| | | | Hydrochloride | | | | | | |
| 5-H10 | Enhancer | C₁₅H₁₆O₄ | Dihydroptaeroxylin | 215783848 | 1.05776396 | 194786588 | 0.95483622 | 1.78E+08 | 204120516 |
| 8-C9 | Enhancer | C₃₁H₄₈O₆ | Fusidic Acid | 229560272 | 1.12529545 | 192354884 | 0.9429161 | 1.78E+08 | 2.04E+08 |
| 18-G4 | Enhancer | C₁₆H₁₄O₃ | Fenbufen | 204633204 | 1.00310394 | 191729340 | 0.93984971 | 1.78E+08 | 2.04E+08 |
| 12-D8 | Enhancer | C₂₈H₃₈O₆ | 3-beta-Hyroxydeoxo- | 213530556 | 0.984012 | 186574276 | 0.859789 | 229622488 | 217454552 |
| | | | dihydrodeoxygedunin | | | | | | |
| 19-C2 | Enhancer | C₂₆H₅₂N₆O₁₁S | Deferoxamine | 194929488 | 0.91516192 | 194519648 | 0.91323778 | 212666320 | 219660188 |
| | | | Mesylate | | | | | | |
| 21-C11 | Enhancer | C₅H₅N₅S | Thioguanine | 181033260 | 0.84992141 | 193528220 | 0.90858319 | 2.13E+08 | 2.11E+08 |
| 24-H11 | Enhancer | C₁₉H₂₄CINO₅ | 2-Aminomethyl-1,4- | 160928732 | 0.9143678 | 137609582 | 0.78187263 | 138649048 | 175962704 |
| | | | Benzodioxane | | | | | | |
| | | | Hydrochloride | | | | | | |
| | | | (WB4101) | | | | | | |
| 2-H8 | Enhancer | C₂₇H₃₆O₇ | 3-alpha-Hydroxy-3- | 128735116 | 100.46% | 118263232 | 91.87% | | |
| | | | Deoxyangolensic | | | | | | |
| | | | acid methyl easter | | | | | | |
| 3-E7 | Enhancer | C₂₅H₃₁N₃O₄ | Lunarine | 159854565 | 81.96% | 130354040 | 81.55% | | |
| 13-G8 | Enhancer | C₃₃H₄₄BrN₅O₈S | Bromocriptine | 120443264 | 109.92% | 136409552 | 113.26% | | |
| | | | Mesylate | | | | | | |

### Example 9

An additional chemical library screen was performed to identify enhancers and suppressors of RNAi activity. In this screen, RNAi-293-EGFP cells were treated with compounds from a chemical library and endogenous RNAi activity was measured by GFP readout. Four particular compounds were identified in this screen. The chemical structures of these compounds are provided in FIG. 10. Further, all four compounds identified in this screen have metal/Fe chelating ability.

These four compounds were further tested by obtaining a drug dose-response curve for each compound at a wide range of concentrations in RNAi-293-EGFP/RFP. After 48 hours, GFP signals were measured by SpectraMax M2^{e} (Molecular Devices Corporation, Sunnyvale, Calif.). The GFP signals were then normalized over RFP signals to account for variability of cell numbers among wells and then plotted for relative RNAi-enhancing activity, which is set to 100% for a known RNAi-enhancing small molecule, e.g., Enoxacin at 50 μM.

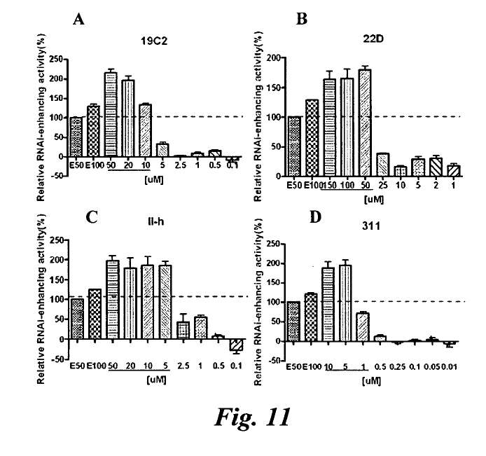

When various dosage responses were tested, each of the four compounds tested exhibited RNAi-enhancing activity that is more effective than Enoxacin (50 μM E50; 100 μM E100), even at low concentrations, for example, 5 μM for compounds designated herein as II-h and 311. See FIG. 11. Moderate to low cell death was observed for these treatments.

Further, when RNAi-293-EGFP/RFP cells were treated with increasing amounts of iron (FAC) ranging from 100 μM to 1 mM, a dose-dependent increase in GFP/RFP signal intensity was observed as compared to untreated cells. This observation is indicative of suppression of RNAi activity. Accordingly, these results suggest that modulation of endogenous RNAi activity can be achieved by alterations in iron homeostasis.

1. A method for decreasing the level of a target polynucleotide in a cell, the method comprising administering to the cell an effective amount of a compound of Formula (a)-(k), or a derivative or analog thereof, wherein said cell further comprises at least one heterologous silencing element:
(a) triprolidine, derivatives, and analogs thereof: 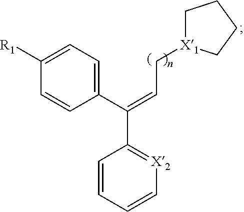
(b) dihydroptaeroxylin, derivatives, and analogs thereof: 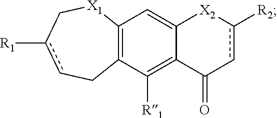
(c) fusidic acid, derivatives, and analogs thereof: 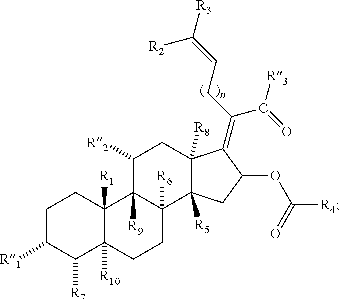
(d) fenbufen, derivatives, and analogs thereof: 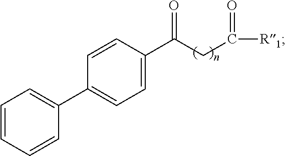
(e) 3-beta-hydroxydeoxyodihydrodeoxygedunin, derivatives, and analogs thereof: 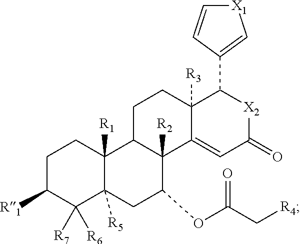
(f) deferoxamine, derivatives, and analogs thereof: 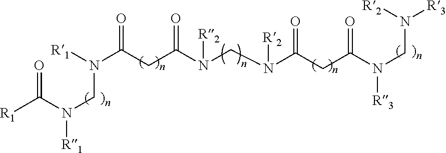
(g) thioguanine, derivatives, and analogs thereof: 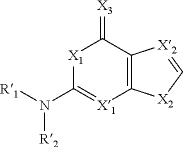
(h) 2-aminomethyl-1,4-benzodioxane, derivatives, and analogs thereof: 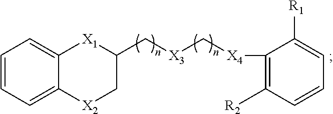
(i) 3-alpha-hydroxy-3-deoxyangolensic acid methyl ester, derivatives, and analogs thereof: 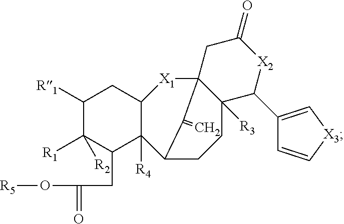
(j) lunarine, derivatives, and analogs thereof: 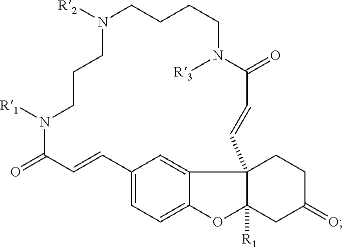
and
(k) bromocriptine, derivatives, and analogs thereof: 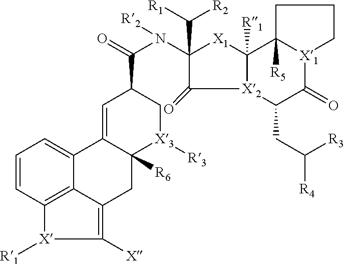
wherein:
each n is independently an integer from 1 to 20;
a dashed line in a cyclic ring structure represents a bond that can be either present or absent in the ring;
each R₁, R₂, R₃, R₄, R₅, R₆, R₇, R₈, R₉, and R₁₀ is independently selected from the group consisting of H, alkyl, substituted alkyl, cycloalkyl, substituted cycloalkyl, aryl, and substituted aryl;
each R′₁, R′₂, and R′₃ is independently selected from the group consisting of H, alkyl, substituted alkyl, cycloalkyl, substituted cycloalkyl, aryl, substituted aryl, hydroxyl, and alkoxyl;
each R″₁, R″₂, R″₃, and R″₄ is independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
each X₁, X₂, X₃, and X₄ is independently selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl;
each X′₁, X′₂, and X′₃ is independently N or CH;
each X″ is independently halogen; and
pharmaceutically and cosmetically acceptable salts thereof.

2. The method of claim 1, wherein the compound of Formula (a)-(k) is selected from the group consisting of triprolidine, dihydropaeroxylin, fusidic acid, fenbufen, 3-beta-hydroxydeoxodihydrodeoxygedunin, deferoxamine, thioguanin, 2-aminomethyl-1,4-benzodioxane, 3-alpha-hydroxy-3-deoxyangloensic acid methyl ester, lunarine, bromocriptine, and pharmaceutically and cosmetically acceptable salts thereof.

3. The method of claim 1, wherein said silencing element comprises an siRNA, an miRNA, a double stranded RNA, or a hairpin RNA.

4. The method of claim 1, wherein said cell is in a subject.

5. A method for decreasing the level of a target polynucleotide in a cell, the method comprising administering to said cell a combination of an effective concentration of a polynucleotide comprising a heterologous silencing element and an effective amount of a compound of Formula (a)-(k), or a derivative or analog thereof:
(a) triprolidine, derivatives, and analogs thereof: 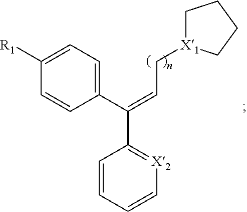
(b) dihydroptaeroxylin, derivatives, and analogs thereof: 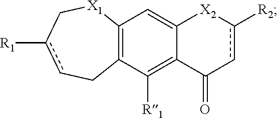
(c) fusidic acid, derivatives, and analogs thereof: 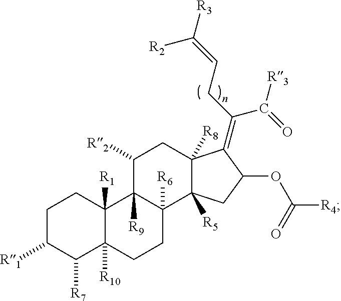
(d) fenbufen, derivatives, and analogs thereof: 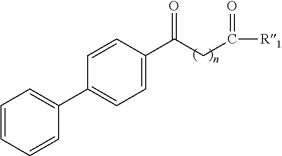
(e) 3-beta-hydroxydeoxyodihydrodeoxygedunin, derivatives, and analogs thereof: 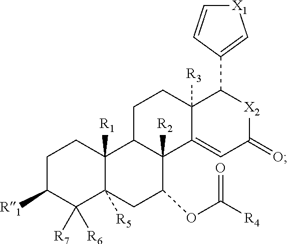
(f) deferoxamine, derivatives, and analogs thereof: 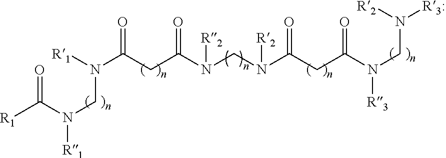
(g) thioguanine, derivatives, and analogs thereof: 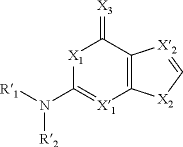
(h) 2-aminomethyl-1,4-benzodioxane, derivatives, and analogs thereof: 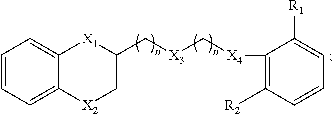
(i) 3-alpha-hydroxy-3-deoxyangolensic acid methyl ester, derivatives, and analogs thereof: 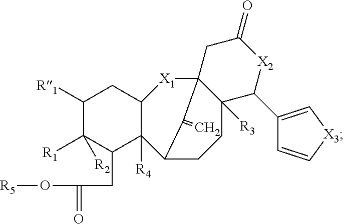
(j) lunarine, derivatives, and analogs thereof: 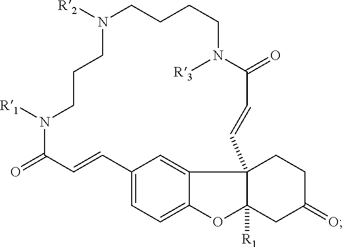
and
(k) bromocriptine, derivatives, and analogs thereof: 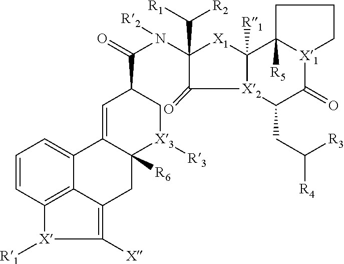
wherein:
each n is independently an integer from 1 to 20;
a dashed line in a cyclic ring structure represents a bond that can be either present or absent in the ring;
each R₁, R₂, R₃, R₄, R₅, R₆, R₇, R₈, R₉, and R₁₀ is independently selected from the group consisting of H, alkyl, substituted alkyl, cycloalkyl, substituted cycloalkyl, aryl, and substituted aryl;
each R′₁, R′₂, and R′₃ is independently selected from the group consisting of H, alkyl, substituted alkyl, cycloalkyl, substituted cycloalkyl, aryl, substituted aryl, hydroxyl, and alkoxyl;
each R″₁, R″₂, and R″₃ is independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
each X₁, X₂, X₃, and X₄ is independently selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl;
each X′₁, X′₂, and X′₃ is independently N or CH;
each X″ is independently halogen; and
pharmaceutically and cosmetically acceptable salts thereof.

6. The method of claim 5, wherein said compound of Formula (a)-(k) is selected from the group consisting of triprolidine, dihydropaeroxylin, fusidic acid, fenbufen, 3-beta-hydroxydeoxodihydrodeoxygedunin, deferoxamine, thioguanin, 2-aminomethyl-1,4-benzodioxane, 3-alpha-hydroxy-3-deoxyangloensic acid methyl ester, lunarine, bromocriptine, and pharmaceutically and cosmetically acceptable salts thereof.

7. The method of claim 5, wherein said polynucleotide comprising the heterologous silencing element comprises an expression cassette encoding a siRNA, a miRNA, a dsRNA, or a hairpin RNA.

8. The method of claim 7, wherein said polynucleotide is in a viral vector.

9. The method of claim 5, wherein said polynucleotide comprises a siRNA, a miRNA, a dsRNA, or a hairpin RNA.

10. The method of claim 5, wherein said compound of Formula (a)-(k) and said heterologous silencing element are administered to the cell simultaneously or sequentially.

11. The method of claim 5, wherein the cell is in a subject.

12. The method of claim 1, wherein said cell is from a mammal.

13. A pharmaceutical or cosmetic composition comprising at least one of a compound of Formula (a)-(k) and a pharmaceutically or cosmetically acceptable carrier and one or more polynucleotides comprising a silencing element.

14. The pharmaceutical or cosmetic composition of claim 13, wherein said silencing element comprises a siRNA, a miRNA, a double stranded RNA, or a hairpin RNA.

15. A method for decreasing the activity of a silencing element in a cell, the method comprising administering to the cell an effective amount of a compound of Formula (l)-(m) or a derivative or analog thereof:
(l) colforsin, derivatives, and analogs thereof: 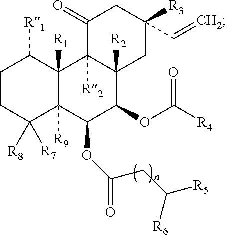
and
(m) metameconine, derivatives, and analogs thereof: 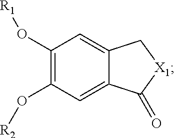
wherein:
n is an integer from 1 to 20;
each R₁, R₂, R₃, R₄, R₅, R₆, R₇, R₈, and R₉ is independently selected from the group consisting of H, alkyl, substituted alkyl, cycloalkyl, substituted cycloalkyl, aryl, and substituted aryl;
R″₁ and R″₂ are each independently selected from the group consisting of —ORᵢᵢ and —O(C═O)—R₁₂, wherein R₁₁ and R₁₂ are selected from the group consisting of H, alkyl, substituted alkyl, cycloalkyl, substituted cycloalkyl, aryl, and substituted aryl;
X₁ is selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl; and
pharmaceutically and cosmetically acceptable salts thereof.

16. The method of claim 15, wherein the compound of Formula (l)-(m) is selected from the group consisting of colforsin and metameconine and pharmaceutically and cosmetically acceptable salts thereof.

17. The method of claim 15, wherein said cell is in a subject.

18. A pharmaceutical or cosmetic composition comprising a compound of Formula (l)-(m), a pharmaceutically or cosmetically acceptable carrier, and one or more polynucleotides comprising a silencing element, which when administered to a subject, decreases the level of a target polynucleotide.

19. A method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a compound of Formula (a)-(k):
(a) triprolidine, derivatives, and analogs thereof: 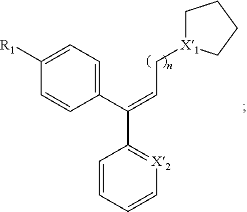
(b) dihydroptaeroxylin, derivatives, and analogs thereof: 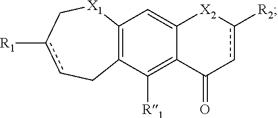
(c) fusidic acid, derivatives, and analogs thereof: 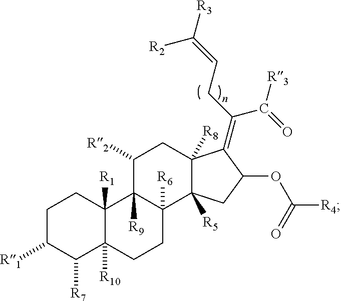
(d) fenbufen, derivatives, and analogs thereof: 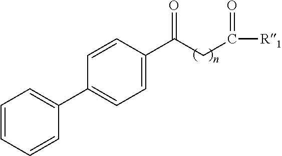
(e) 3-beta-hydroxydeoxyodihydrodeoxygedunin, derivatives, and analogs thereof: 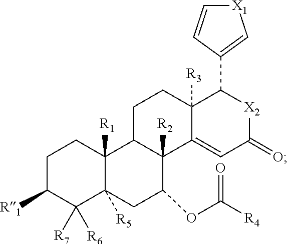
(f) deferoxamine, derivatives, and analogs thereof: 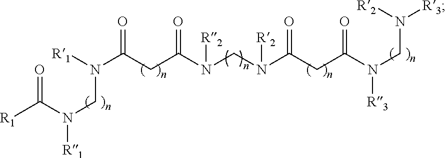
(g) thioguanine, derivatives, and analogs thereof: 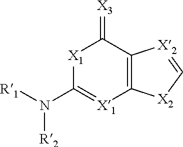
(h) 2-aminomethyl-1,4-benzodioxane, derivatives, and analogs thereof: 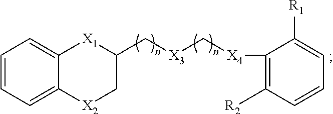
(i) 3-alpha-hydroxy-3-deoxyangolensic acid methyl ester, derivatives, and analogs thereof: 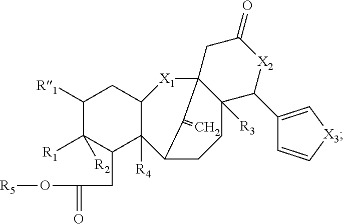
(j) lunarine, derivatives, and analogs thereof: 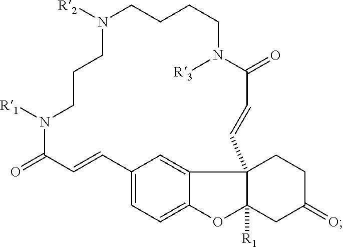
and
(k) bromocriptine, derivatives, and analogs thereof: 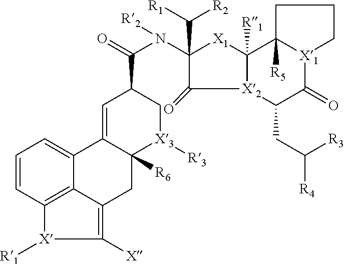
wherein:
each n is independently an integer from 1 to 20;
a dashed line in a cyclic ring structure represents a bond that can be either present or absent in the ring;
each R₁, R₂, R₃, R₄, R₅, R₆, R₇, R₈, R₉, and R₁₀ is independently selected from the group consisting of H, alkyl, substituted alkyl, cycloalkyl, substituted cycloalkyl, aryl, and substituted aryl;
each R′₁, R′₂, and R′₃ is independently selected from the group consisting of H, alkyl, substituted alkyl, cycloalkyl, substituted cycloalkyl, aryl, substituted aryl, hydroxyl, and alkoxyl;
each R″₁, R″₂, and R″₃ is independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
each X₁, X₂, X₃, and X₄ is independently selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl;
each X′₁, X′₂, and X′₃ is independently N or CH;
each X″ is independently halogen; and
pharmaceutically and cosmetically acceptable salts thereof.

20. The method of claim 19, wherein the compound of Formula (a)-(k) is selected from the group consisting of triprolidine, dihydropaeroxylin, fusidic acid, fenbufen, 3-beta-hydroxydeoxodihydrodeoxygedunin, deferoxamine, thioguanin, 2-aminomethyl-1,4-benzodioxane, 3-alpha-hydroxy-3-deoxyangloensic acid methyl ester, lunarine, bromocriptine, and pharmaceutically and cosmetically acceptable salts thereof.

21. The method of claim 19, wherein said disease state comprises a viral infection.

22. The method of claim 19, wherein said disease state comprises a genetic disorder.

23. A method for treating a disease state or unwanted condition, the method comprising administering to a subject in need of treatment thereof an effective amount of a polynucleotide comprising a silencing element, which, when administered to said subject, decreases the level of the target polynucleotide and an effective amount of at least one compound of Formula (l)-(m):
(l) colforsin, derivatives, and analogs thereof: 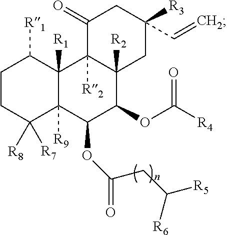
and
(m) metameconine, derivatives, and analogs thereof: 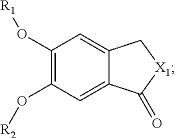
wherein:
n is an integer from 1 to 20;
each R₁, R₂, R₃, R₄, R₅, R₆, R₇, R₈, and R₉ is independently selected from the group consisting of H, alkyl, substituted alkyl, cycloalkyl, substituted cycloalkyl, aryl, and substituted aryl;
R″₁ and R″₂ are each independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
X₁ is selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl; and
pharmaceutically and cosmetically acceptable salts thereof.

24. The method of claim 23, wherein said disease state comprises a viral infection.

25. The method of claim 23, wherein said disease state comprises a genetic disorder.

26. A method for decreasing the level of a target polynucleotide in a cell, the method comprising administering to the cell a combination of an effective concentration of a polynucleotide comprising a heterologous silencing element and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is selected from the group consisting of a compound of Formula (a)-(k), and derivatives and analogs thereof, wherein said cell further comprises at least one heterologous silencing element:
(a) triprolidine, derivatives, and analogs thereof: 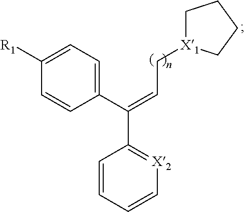
(b) dihydroptaeroxylin, derivatives, and analogs thereof: 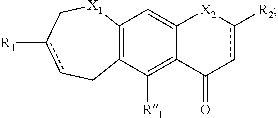
(c) fusidic acid, derivatives, and analogs thereof: 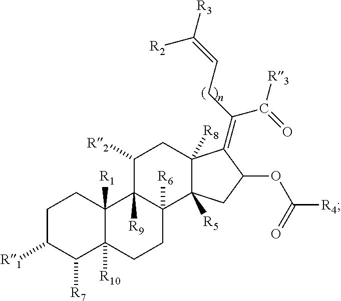
(d) fenbufen, derivatives, and analogs thereof: 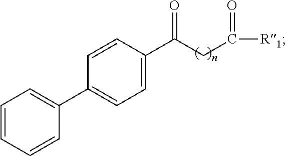
(e) 3-beta-hydroxydeoxyodihydrodeoxygedunin, derivatives, and analogs thereof: 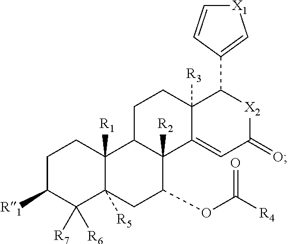
(f) deferoxamine, derivatives, and analogs thereof: 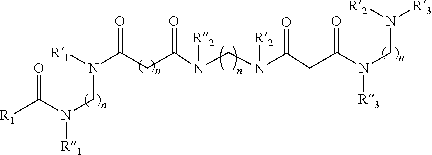
(g) thioguanine, derivatives, and analogs thereof: 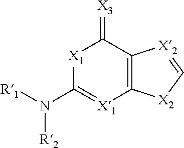
(h) 2-aminomethyl-1,4-benzodioxane, derivatives, and analogs thereof: 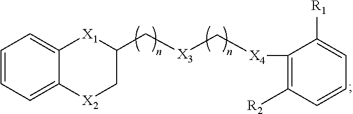
(i) 3-alpha-hydroxy-3-deoxyangolensic acid methyl ester, derivatives, and analogs thereof: 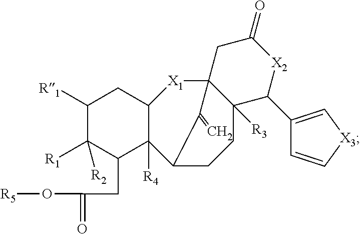
(j) lunarine, derivatives, and analogs thereof: 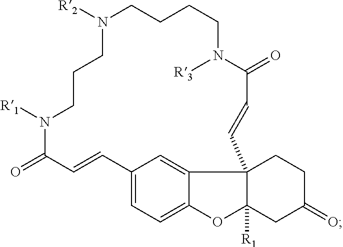
and
(k) bromocriptine, derivatives, and analogs thereof: 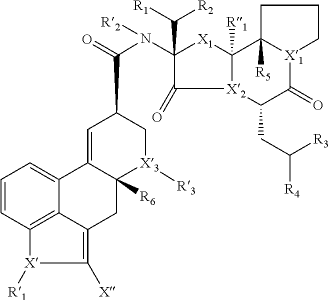
wherein:
each n is independently an integer from 1 to 20;
a dashed line in a cyclic ring structure represents a bond that can be either present or absent in the ring;
each R₁, R₂, R₃, R₄, R₅, R₆, R₇, R₈, R₉, and R₁₀ is independently selected from the group consisting of H, alkyl, substituted alkyl, cycloalkyl, substituted cycloalkyl, aryl, and substituted aryl;
each R′₁, R′₂, and R′₃ is independently selected from the group consisting of H, alkyl, substituted alkyl, cycloalkyl, substituted cycloalkyl, aryl, substituted aryl, hydroxyl, and alkoxyl;
each R″₁, R″₂, R″₃, and R″₄ is independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
each X₁, X₂, X₃, and X₄ is independently selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl;
each X′₁, X′₂, and X′₃ is independently N or CH;
each X″ is independently halogen; and
pharmaceutically and cosmetically acceptable salts thereof.

27. The method of claim 26, wherein the compound of Formula (a)-(k) is selected from the group consisting of triprolidine, dihydroptaeroxylin, fusidic acid, fenbufen, 3-beta-hydroxydeoxodihydrodeoxygedunin, deferoxamine, thioguanin, 2-aminomethyl-1,4-benzodioxane, 3-alpha-hydroxy-3-deoxyangloensic acid methyl ester, lunarine, bromocriptine, and pharmaceutically and cosmetically acceptable salts thereof.

28. The method of claim 26, wherein the RNAi enhancer comprises enoxacin, or a derivative or analog thereof.

29. The method of claim 26, wherein said silencing element comprises an siRNA, an miRNA, a double stranded RNA, or a hairpin RNA.

30. The method of claim 26, wherein said cell is in a subject.

31. A method for decreasing the level of a target polynucleotide in a cell, the method comprising administering to said cell a combination of an effective concentration of a polynucleotide comprising a heterologous silencing element and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is selected from the group consisting of a compound of Formula (a)-(k), and derivatives and analogs thereof:
(a) triprolidine, derivatives, and analogs thereof: 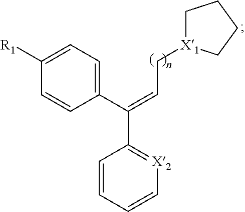
(b) dihydroptaeroxylin, derivatives, and analogs thereof: 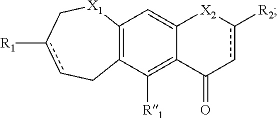
(c) fusidic acid, derivatives, and analogs thereof: 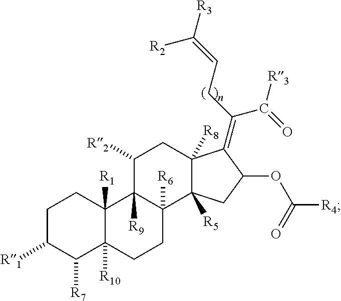
(d) fenbufen, derivatives, and analogs thereof: 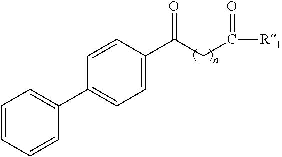
(e) 3-beta-hydroxydeoxyodihydrodeoxygedunin, derivatives, and analogs thereof: 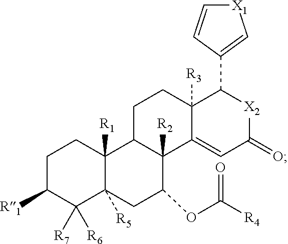
(f) deferoxamine, derivatives, and analogs thereof: 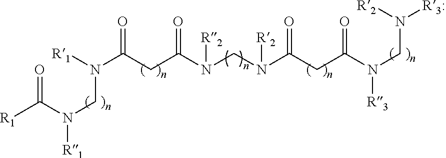
(g) thioguanine, derivatives, and analogs thereof: 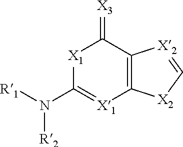
(h) 2-aminomethyl-1,4-benzodioxane, derivatives, and analogs thereof: 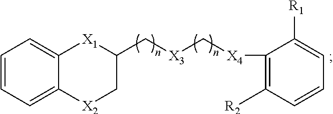
(i) 3-alpha-hydroxy-3-deoxyangolensic acid methyl ester, derivatives, and analogs thereof: 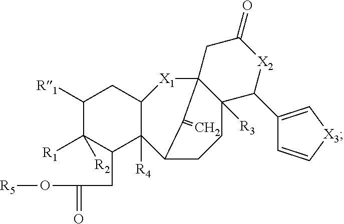
(j) lunarine, derivatives, and analogs thereof: 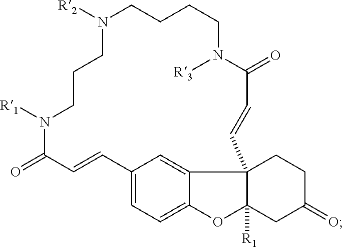
and
(k) bromocriptine, derivatives, and analogs thereof: 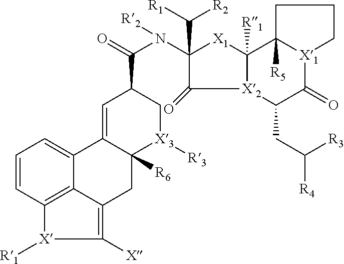
wherein:
each n is independently an integer from 1 to 20;
a dashed line in a cyclic ring structure represents a bond that can be either present or absent in the ring;
each R₁, R₂, R₃, R₄, R₅, R₆, R₇, R₈, R₉, and R₁₀ is independently selected from the group consisting of H, alkyl, substituted alkyl, cycloalkyl, substituted cycloalkyl, aryl, and substituted aryl;
each R′₁, R′₂, and R′₃ is independently selected from the group consisting of H, alkyl, substituted alkyl, cycloalkyl, substituted cycloalkyl, aryl, substituted aryl, hydroxyl, and alkoxyl;
each R″₁, R″₂, and R″₃ is independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
each X₁, X₂, X₃, and X₄ is independently selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl;
each X′₁, X′₂, and X′₃ is independently N or CH;
each X″ is independently halogen; and
pharmaceutically and cosmetically acceptable salts thereof.

32. The method of claim 31, wherein said compound of Formula (a)-(k) is selected from the group consisting of triprolidine, dihydroptaeroxylin, fusidic acid, fenbufen, 3-beta-hydroxydeoxodihydrodeoxygedunin, deferoxamine, thioguanin, 2-aminomethyl-1,4-benzodioxane, 3-alpha-hydroxy-3-deoxyangloensic acid methyl ester, lunarine, bromocriptine, and pharmaceutically and cosmetically acceptable salts thereof.

33. The method of claim 32, wherein at least one of the first RNAi enhancer and the at least one second RNAi enhancer comprises enoxacin, or a derivative or analog thereof.

34. The method of claim 29, wherein said polynucleotide comprising the heterologous silencing element comprises an expression cassette encoding a siRNA, a miRNA, a dsRNA, or a hairpin RNA.

35. The method of claim 29, wherein said polynucleotide is in a viral vector.

36. The method of claim 29, wherein said polynucleotide comprises a siRNA, a miRNA, a dsRNA, or a hairpin RNA.

37. The method of claim 29, wherein said the first RNAi enhancer and the at least one second RNAi enhancer and said heterologous silencing element are administered to the cell simultaneously or sequentially.

38. The method of claim 29, wherein the cell is in a subject.

39. The method of claim 26, wherein said cell is from a mammal.

40. A pharmaceutical or cosmetic composition comprising at a first RNAi enhancer in combination with at least one second RNAi enhancer, wherein at least one of the first RNAi enhancer and the second RNAi enhancer is selected from the group consisting of a compound of Formula (a)-(k), and derivatives and analogs thereof, and a pharmaceutically or cosmetically acceptable carrier and one or more polynucleotides comprising a silencing element.

41. The pharmaceutical or cosmetic composition of claim 40, wherein said silencing element comprises a siRNA, a miRNA, a double stranded RNA, or a hairpin RNA.

42. A method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is selected from the group consisting of a compound of Formula (a)-(k), and derivatives and analogs thereof:
(a) triprolidine, derivatives, and analogs thereof: 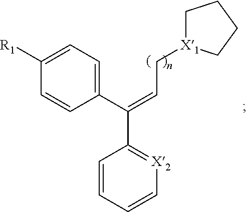
(b) dihydroptaeroxylin, derivatives, and analogs thereof: 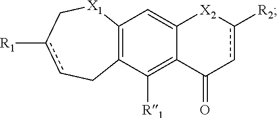
(c) fusidic acid, derivatives, and analogs thereof: 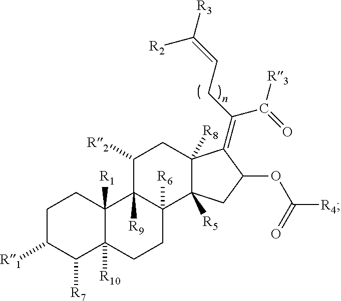
(d) fenbufen, derivatives, and analogs thereof: 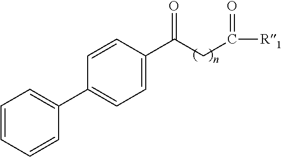
(e) 3-beta-hydroxydeoxyodihydrodeoxygedunin, derivatives, and analogs thereof: 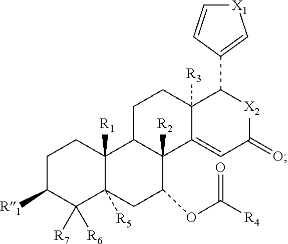
(f) deferoxamine, derivatives, and analogs thereof: 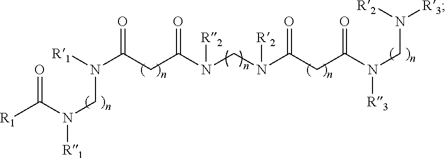
(g) thioguanine, derivatives, and analogs thereof: 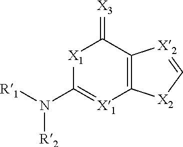
(h) 2-aminomethyl-1,4-benzodioxane, derivatives, and analogs thereof: 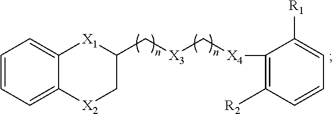
(i) 3-alpha-hydroxy-3-deoxyangolensic acid methyl ester, derivatives, and analogs thereof: 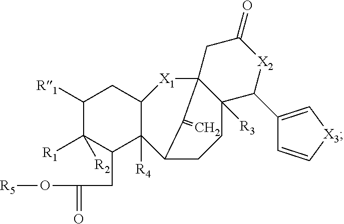
(j) lunarine, derivatives, and analogs thereof: 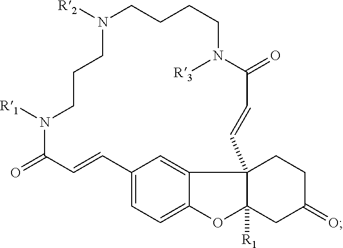
and
(k) bromocriptine, derivatives, and analogs thereof: 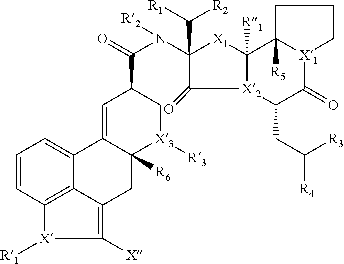
wherein:
each n is independently an integer from 1 to 20;
a dashed line in a cyclic ring structure represents a bond that can be either present or absent in the ring;
each R₁, R₂, R₃, R₄, R₅, R₆, R₇, R₈, R₉, and R₁₀ is independently selected from the group consisting of H, alkyl, substituted alkyl, cycloalkyl, substituted cycloalkyl, aryl, and substituted aryl;
each R′₁, R′₂, and R′₃ is independently selected from the group consisting of H, alkyl, substituted alkyl, cycloalkyl, substituted cycloalkyl, aryl, substituted aryl, hydroxyl, and alkoxyl;
each R″₁, R″₂, and R″₃ is independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
each X₁, X₂, X₃, and X₄ is independently selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl;
each X′₁, X′₂, and X′₃ is independently N or CH;
each X″ is independently halogen; and
pharmaceutically and cosmetically acceptable salts thereof.

43. The method of claim 42, wherein the compound of Formula (a)-(k) is selected from the group consisting of triprolidine, dihydroptaeroxylin, fusidic acid, fenbufen, 3-beta-hydroxydeoxodihydrodeoxygedunin, deferoxamine, thioguanin, 2-aminomethyl-1,4-benzodioxane, 3-alpha-hydroxy-3-deoxyangloensic acid methyl ester, lunarine, bromocriptine, and pharmaceutically and cosmetically acceptable salts thereof.

44. The method of claim 42, wherein at least one of the first RNAi enhancer and the at least one second RNAi enhancer comprises enoxacin, or derivatives or analogs thereof.

45. The method of claim 42, wherein said disease state comprises a viral infection.

46. The method of claim 42, wherein said disease state comprises a genetic disorder.

47. A method for screening a compound of interest for the ability to modulate the activity of a heterologous silencing element comprising:
a) providing a host cell that stably expresses a reporter gene, wherein said host cell further comprises at least one heterologous silencing element capable of inhibiting the expression of the reporter gene;
b) administering to the cell a compound of interest in the presence of an RNAi enhancer; and
c) measuring the expression of the reporter gene.

48. The method of claim 47, wherein the RNAi enhancer comprises enoxacin, or derivatives or analogs thereof.

49. The method of claim 47, wherein said silencing element comprises an siRNA, an miRNA, a double stranded RNA, or a hairpin RNA.

50. The method of claim 47 wherein said reporter gene encodes green fluorescent protein.

51. A method for modulating the activity of a silencing element in a cell, the method comprising administering to the cell an effective amount of at least one RNAi enhancer and an effective amount of at least one compound of Formula (l)-(m) or a derivative or analog thereof, to modulate the activity of said silencing element:
(l) colforsin, derivatives, and analogs thereof: 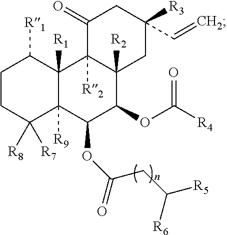
and
(m) metameconine, derivatives, and analogs thereof: 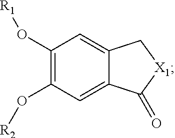
wherein:
n is an integer from 1 to 20;
each R₁, R₂, R₃, R₄, R₅, R₆, R₇, R₈, and R₉ is independently selected from the group consisting of H, alkyl, substituted alkyl, cycloalkyl, substituted cycloalkyl, aryl, and substituted aryl;
R″₁ and R″₂ are each independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
X₁ is selected from the group consisting of CH₂, O, S, and NR′₄, wherein R′₄ is selected from the group consisting of H, alkyl, substituted alkyl, cycloalkyl, substituted cycloalkyl, aryl, substituted aryl, hydroxyl, and alkoxyl; and
pharmaceutically and cosmetically acceptable salts thereof.

52. The method of claim 51, wherein the at least one compound of Formula (l)-(m) is selected from the group consisting of colforsin and metameconine and pharmaceutically and cosmetically acceptable salts thereof.

53. The method of claim 51, wherein said cell is in a subject.

54. The method of claim 51, wherein the at least one RNAi enhancer comprises enoxacin, or a derivative or analog thereof.

55. A pharmaceutical or cosmetic composition comprising at least one RNAi enhancer in combination with at least one RNAi inhibitor, wherein the at least one of RNAi inhibitor is selected from the group consisting of a compound of Formula (l)-(m), and derivatives and analogs thereof, and a pharmaceutically or cosmetically acceptable carrier and one or more polynucleotides comprising a silencing element.

56. A method for treating a disease state or unwanted condition, the method comprising administering to a subject in need of treatment thereof an effective amount of a polynucleotide comprising a silencing element, which, when administered to said subject, decreases the level of the target polynucleotide and an effective amount of at least one compound of Formula (l)-(m) and at least one RNAi enhancer:
(l) colforsin, derivatives, and analogs thereof: 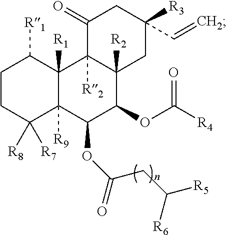
and
(m) metameconine, derivatives, and analogs thereof: 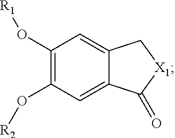
wherein:
n is an integer from 1 to 20;
each R₁, R₂, R₃, R₄, R₅, R₆, R₇, R₈, and R₉ is independently selected from the group consisting of H, alkyl, substituted alkyl, cycloalkyl, substituted cycloalkyl, aryl, and substituted aryl;
R″₁ and R″₂ are each independently selected from the group consisting of —OR₁₁ and —O(C═O)—R₁₂, wherein R₁₁ and R₁₂ are selected from the group consisting of H, alkyl, substituted alkyl, cycloalkyl, substituted cycloalkyl, aryl, and substituted aryl;
X₁ is selected from the group consisting of CH₂, O, S, and NR'₄, wherein R′₄ is selected from the group consisting of H, alkyl, substituted alkyl, cycloalkyl, substituted cycloalkyl, aryl, substituted aryl, hydroxyl, and alkoxyl; and
pharmaceutically and cosmetically acceptable salts thereof.

57. The method of claim 56, wherein said disease state comprises a viral infection.

58. The method of claim 56, wherein said disease state comprises a genetic disorder.

59. The method of claim 56, wherein said RNAi enhancer comprises enoxacin, or a derivative or analog thereof.

60. A method for decreasing the level of a target polynucleotide in a cell, the method comprising administering to the cell an effective amount of an iron chelating agent, and pharmaceutically and cosmetically acceptable salts thereof, wherein said cell further comprises at least one heterologous silencing element.

61. The method of claim 60, wherein the iron chelating agent is selected from a compound of Formula (n)-(q): 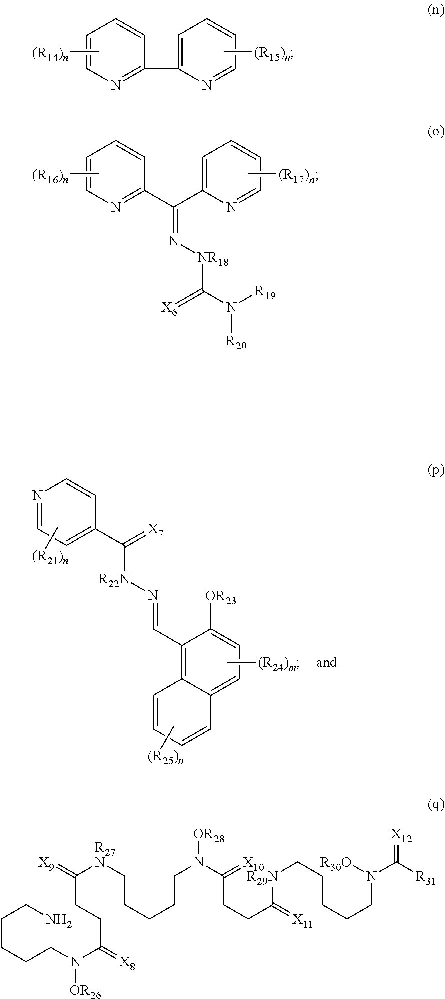
wherein:
m is an integer from 0 to 3;
n is an integer from 0 to 4;
R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;
R₁₈, R₂₂, R₂₇, and R₂₉ are each independently selected from the group consisting of H, alkyl, substituted alkyl, and hydroxyl;
R₂₃, R₂₆, R₂₈, and R₃₀ are each independently selected from the group consisting of H, alkyl, and substituted alkyl;
R₁₉, R₂₀, and R₃₁ are each independently selected from the group consisting of H, alkyl, substituted alkyl;
X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and
pharmaceutically and cosmetically acceptable salts thereof.

62. The method of claim 61, wherein the compound of Formula (n)-(q) is selected from the group consisting of: 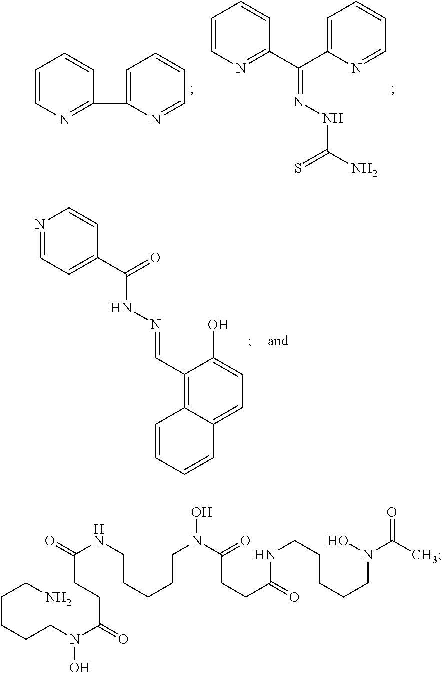
and
pharmaceutically and cosmetically acceptable salts thereof.

63. The method of claim 60, wherein said silencing element comprises an siRNA, an miRNA, a double stranded RNA, or a hairpin RNA.

64. The method of claim 60, wherein said cell is in a subject.

65. A method for decreasing the level of a target polynucleotide in a cell, the method comprising administering to said cell a combination of an effective concentration of a polynucleotide comprising a heterologous silencing element and an effective amount of an iron chelating agent, and pharmaceutically and cosmetically acceptable salts thereof.

66. The method of claim 65, wherein the iron chelating agent is selected from a compound of Formula (n)-(q): 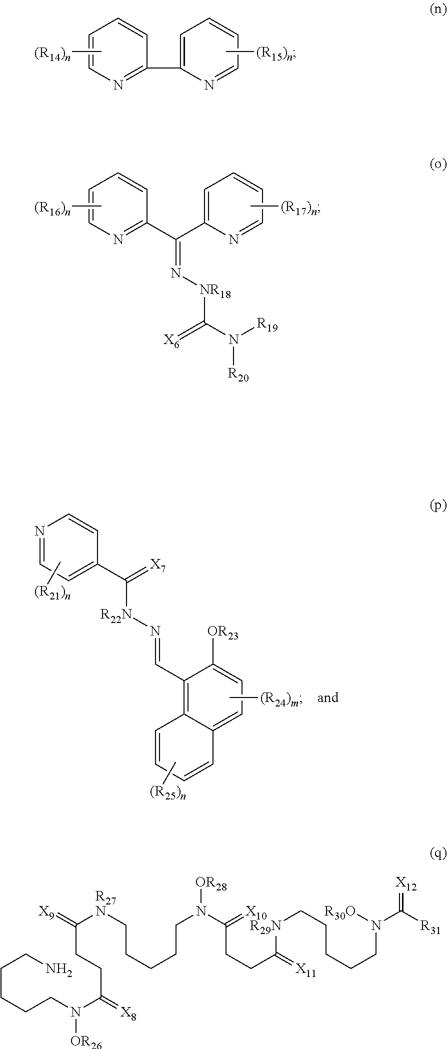
wherein:
m is an integer from 0 to 3;
n is an integer from 0 to 4;
R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;
R₁₈, R₂₂, R₂₇, and R₂₉ are each independently selected from the group consisting of H, alkyl, substituted alkyl, and hydroxyl;
R₂₃, R₂₆, R₂₈, and R₃₀ are each independently selected from the group consisting of H, alkyl, and substituted alkyl;
R₁₉, R₂₀, and R₃₁ are each independently selected from the group consisting of H, alkyl, substituted alkyl;
X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and
pharmaceutically and cosmetically acceptable salts thereof.

67. The method of claim 66, wherein said compound of Formula (n)-(q) is selected from the group consisting of 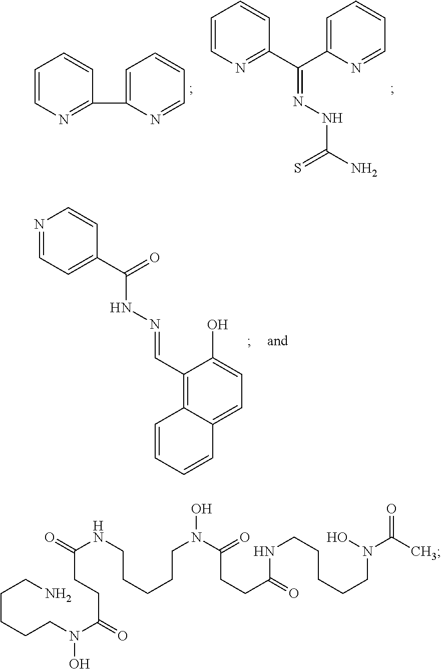
and
pharmaceutically and cosmetically acceptable salts thereof.

68. The method of claim 65, wherein said polynucleotide comprising the heterologous silencing element comprises an expression cassette encoding a siRNA, a miRNA, a dsRNA, or a hairpin RNA.

69. The method of claim 68, wherein said polynucleotide is in a viral vector.

70. The method of claim 65, wherein said polynucleotide comprises a siRNA, a miRNA, a dsRNA, or a hairpin RNA.

71. The method of claim 65, wherein said iron chelating agent and said heterologous silencing element are administered to the cell simultaneously or sequentially.

72. The method of claim 65, wherein the cell is in a subject.

73. The method of claim 60, wherein said cell is from a mammal.

74. A pharmaceutical or cosmetic composition comprising at least one of a compound of Formula (n)-(q) and a pharmaceutically or cosmetically acceptable carrier and one or more polynucleotides comprising a silencing element.

75. The pharmaceutical or cosmetic composition of claim 74, wherein said silencing element comprises a siRNA, a miRNA, a double stranded RNA, or a hairpin RNA.

76. A method for treating a disease state or unwanted condition, the method comprising administering to a subject in need of treatment thereof an effective amount of a polynucleotide comprising a silencing element, which, when administered to said subject, decreases the level of the target polynucleotide and an effective amount of an iron chelating agent, and pharmaceutically and cosmetically acceptable salts thereof.

77. The method of claim 76, wherein the iron chelating agent is selected from a compound of Formula (n)-(q): 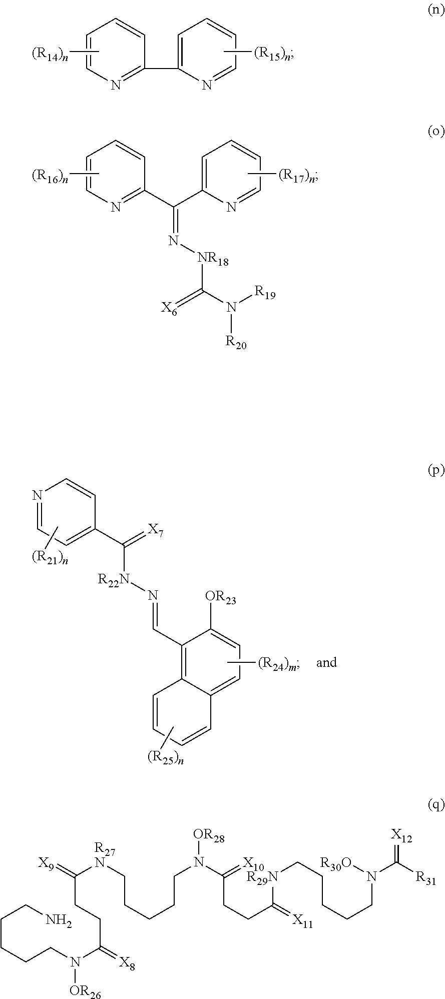
wherein:
m is an integer from 0 to 3;
n is an integer from 0 to 4;
R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;
R₁₈, R₂₂, R₂₇, and R₂₉ are each independently selected from the group consisting of H, alkyl, substituted alkyl, and hydroxyl;
R₂₃, R₂₆, R₂₈, and R₃₀ are each independently selected from the group consisting of H, alkyl, and substituted alkyl;
R₁₉, R₂₀, and R₃₁ are each independently selected from the group consisting of H, alkyl, substituted alkyl;
X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and
pharmaceutically and cosmetically acceptable salts thereof.

78. The method of claim 77, wherein the compound of Formula (n)-(q) is selected from the group consisting of: 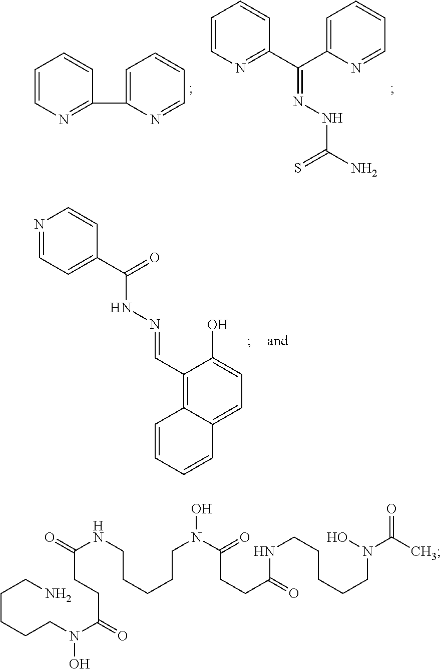
and
pharmaceutically and cosmetically acceptable salts thereof.

79. The method of claim 76, wherein said disease state is a cancer.

80. The method of claim 79, wherein the cancer is selected from the group consisting of neuroblastoma (NB) and leukemia.

81. The method of claim 76, wherein said disease state comprises a neurodegenerative disorder.

82. The method of claim 81, wherein the neurodegenerative disease is selected from the group consisting of Huntington disease, Alzheimer's disease, Parkinson's disease, Friedreich's atazia, hereditary hyperferritinemia cataract syndrome, X-linked siderablastic anemia, and progressive neurodegenerative disease.

83. A method for decreasing the level of a target polynucleotide in a cell, the method comprising administering to the cell a combination of an effective concentration of a polynucleotide comprising a heterologous silencing element and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is an iron chelating agent, and pharmaceutically and cosmetically acceptable salts thereof, wherein said cell further comprises at least one heterologous silencing element.

84. The method of claim 83, wherein the iron chelating agent is selected from a compound of Formula (n)-(q): 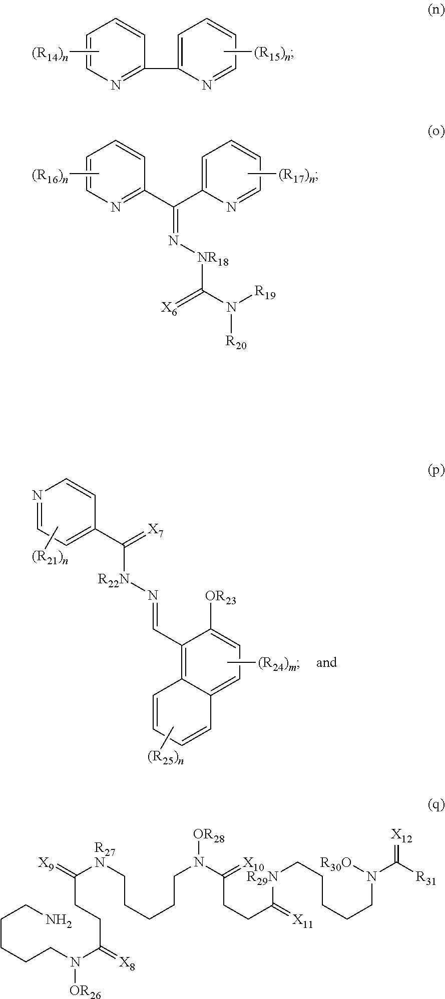
wherein:
m is an integer from 0 to 3;
n is an integer from 0 to 4;
R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;
R₁₈, R₂₂, R₂₇, and R₂₉ are each independently selected from the group consisting of H, alkyl, substituted alkyl, and hydroxyl;
R₂₃, R₂₆, R₂₈, and R₃₀ are each independently selected from the group consisting of H, alkyl, and substituted alkyl;
R₁₉, R₂₀, and R₃₁ are each independently selected from the group consisting of H, alkyl, substituted alkyl;
X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and
pharmaceutically and cosmetically acceptable salts thereof.

85. The method of claim 84, wherein the compound of Formula (n)-(q) is selected from the group consisting of: 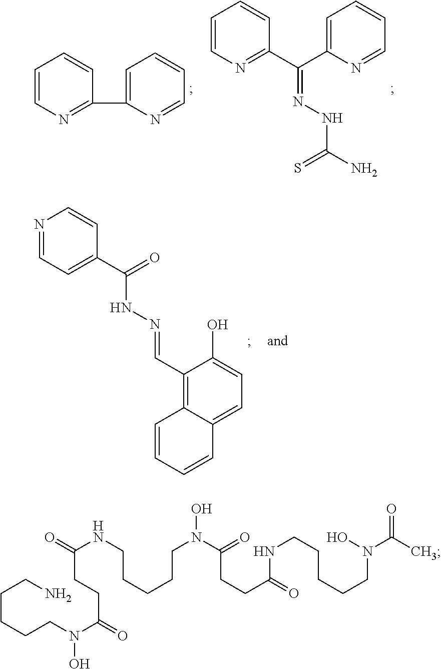
and
pharmaceutically and cosmetically acceptable salts thereof.

86. The method of claim 83, wherein the RNAi enhancer comprises enoxacin, or a derivative or analog thereof.

87. The method of claim 83, wherein said silencing element comprises an siRNA, an miRNA, a double stranded RNA, or a hairpin RNA.

88. The method of claim 83, wherein said cell is in a subject.

89. A method for decreasing the level of a target polynucleotide in a cell, the method comprising administering to said cell a combination of an effective concentration of a polynucleotide comprising a heterologous silencing element and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is an iron chelating agent, and pharmaceutically and cosmetically acceptable salts thereof.

90. The method of claim 89, wherein the iron chelating agent is selected from a compound of Formula (n)-(q): 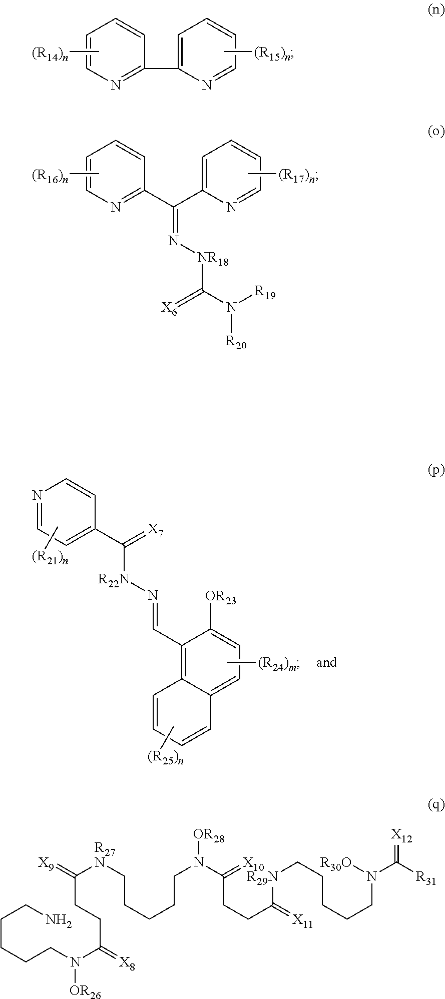
wherein:
m is an integer from 0 to 3;
n is an integer from 0 to 4;
R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;
R₁₈, R₂₂, R₂₇, and R₂₉ are each independently selected from the group consisting of H, alkyl, substituted alkyl, and hydroxyl;
R₂₃, R₂₆, R₂₈, and R₃₀ are each independently selected from the group consisting of H, alkyl, and substituted alkyl;
R₁₉, R₂₀, and R₃₁ are each independently selected from the group consisting of H, alkyl, substituted alkyl;
X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and
pharmaceutically and cosmetically acceptable salts thereof.

91. The method of claim 90, wherein the compound of Formula (n)-(q) is selected from the group consisting of: 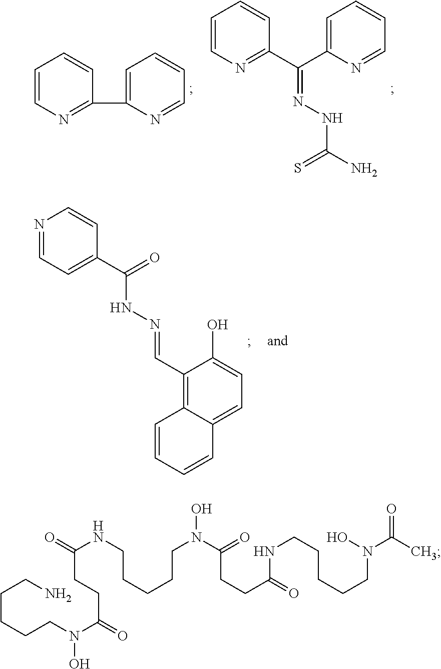
and
pharmaceutically and cosmetically acceptable salts thereof.

92. The method of claim 89, wherein at least one of the first RNAi enhancer and the at least one second RNAi enhancer comprises enoxacin, or a derivative or analog thereof.

93. The method of claim 89, wherein said polynucleotide comprising the heterologous silencing element comprises an expression cassette encoding a siRNA, a miRNA, a dsRNA, or a hairpin RNA.

94. The method of claim 89, wherein said polynucleotide is in a viral vector.

95. The method of claim 89, wherein said polynucleotide comprises a siRNA, a miRNA, a dsRNA, or a hairpin RNA.

96. The method of claim 89, wherein said the first RNAi enhancer and the at least one second RNAi enhancer and said heterologous silencing element are administered to the cell simultaneously or sequentially.

97. The method of claim 89, wherein the cell is in a subject.

98. The method of claim 83, wherein said cell is from a mammal.

99. A pharmaceutical or cosmetic composition comprising at a first RNAi enhancer in combination with at least one second RNAi enhancer, wherein at least one of the first RNAi enhancer and the second RNAi enhancer is selected from the group consisting of a compound of Formula (n)-(q), and derivatives and analogs thereof, and a pharmaceutically or cosmetically acceptable carrier and one or more polynucleotides comprising a silencing element.

100. The pharmaceutical or cosmetic composition of claim 99, wherein said silencing element comprises a siRNA, a miRNA, a double stranded RNA, or a hairpin RNA.

101. A method for treating a disease state or unwanted condition, the method comprising administering to a subject in need of treatment thereof an effective amount of a polynucleotide comprising a silencing element, which, when administered to said subject, decreases the level of the target polynucleotide and an effective amount of a first RNAi enhancer in combination with an effective amount of at least one second RNAi enhancer, wherein the combination of the first RNAi enhancer and at least one second RNAi enhancer synergistically decreases the level of the target polynucleotide in the cell, and wherein at least one of the first RNAi enhancer and the second RNAi enhancer is an iron chelating agent, and pharmaceutically and cosmetically acceptable salts thereof.

102. The method of claim 101, wherein the iron chelating agent is selected from a compound of Formula (n)-(q): 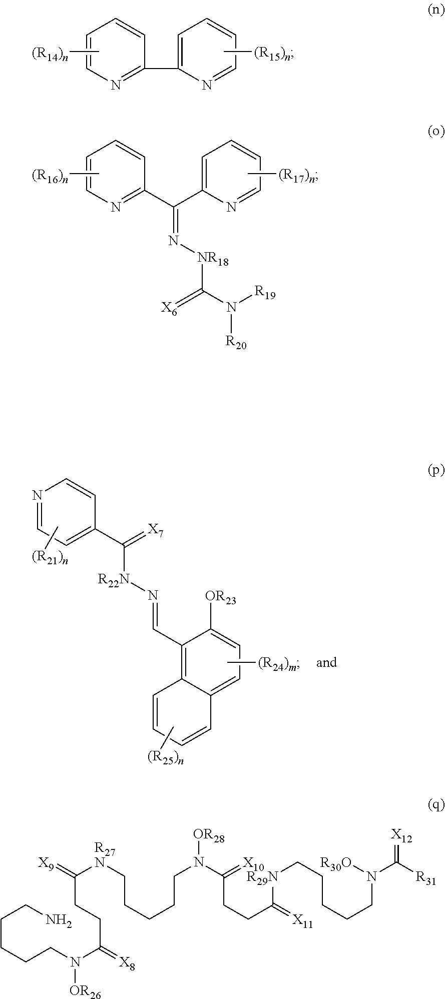
wherein:
m is an integer from 0 to 3;
n is an integer from 0 to 4;
R₁₄, R₁₅, R₁₆, R₁₇, R₂₁, R₂₄, and R₂₅ are each independently selected from the group consisting of H, alkyl, substituted alkyl, cycloalkyl, substituted cycloalkyl, heteroalkyl, substituted heteroalkyl, aryl, substituted aryl, heteroaryl, substituted heteroaryl, hydroxyl, alkoxyl, amino, and halo;
R₁₈, R₂₂, R₂₇, and R₂₉ are each independently selected from the group consisting of H, alkyl, substituted alkyl, and hydroxyl;
R₂₃, R₂₆, R₂₈, and R₃₀ are each independently selected from the group consisting of H, alkyl, and substituted alkyl;
R₁₉, R₂₀, and R₃₁ are each independently selected from the group consisting of H, alkyl, substituted alkyl;
X₆, X₇, X₈, X₉, X₁₀, X₁₁, and X₁₂ are each independently selected from the group consisting of O, S, and NR₃₂, wherein R₃₂ is selected from the group consisting of H, alkyl, substituted alkyl, and hydroxyl; and
pharmaceutically and cosmetically acceptable salts thereof.

103. The method of claim 102, wherein the compound of Formula (n)-(q) is selected from the group consisting of: 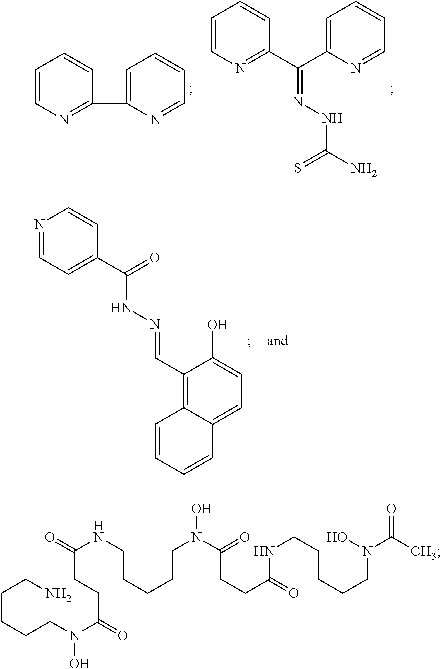
and
pharmaceutically and cosmetically acceptable salts thereof.

104. The method of claim 101, wherein at least one of the first RNAi enhancer and the at least one second RNAi enhancer comprises enoxacin, or derivatives or analogs thereof.

105. The method of claim 101, wherein said disease state is a cancer.

106. The method of claim 105, wherein the cancer is selected from the group consisting of neuroblastoma (NB) and leukemia.

107. The method of claim 101, wherein said disease state comprises a neurodegenerative disorder.

108. The method of claim 107, wherein the neurodegenerative disease is selected from the group consisting of Huntington disease, Alzheimer's disease, Parkinson's disease, Friedreich's atazia, hereditary hyperferritinemia cataract syndrome, X-linked siderablastic anemia, and progressive neurodegenerative disease.
